(19) United States
(12) Patent Application Publication
(10) Pub. No.: US 2025/0213581 A1
(43) Pub. Date: Jul. 3, 2025

(54) COMBINATION THERAPY OF A PI3K INHIBITOR AND KRAS INHIBITOR AND METHODS OF USE THEREOF

(71) Applicant: Totus Medicines Inc., Emeryville, CA (US)

(72) Inventors: John MACDOUGALL, Hingham, MA (US); Neil Sonin DHAWAN, Somerville, MA (US); James Abellera BLAIR, Arlington, MA (US); Robert B. PERNI, Marlborough, MA (US)

(21) Appl. No.: 18/849,949

(22) PCT Filed: Mar. 24, 2023
(86) PCT No.: PCT/US2023/064954

§ 371 Date: Sep. 23, 2024

Related U.S. Application Data
(60) Provisional application No. 63/323,358, filed on Mar. 24, 2022.

(51) Int. Cl. A61K 31/5377; A61K 31/519; A61P 35/00

(52) U.S. Cl. CPC A61K 31/5377; A61K 31/519; A61P 35/00

(57) ABSTRACT

The present invention provides methods, compositions, and combinations for treating cancer via combined use of a compound that forms a covalent bond with a kinase or pseudokinase, or a pharmaceutically acceptable salt thereof, and at least one additional therapeutically active agent.

### CROSS-REFERENCE TO RELATED APPLICATIONS

This application claims priority to U.S. Provisional Patent Application No. 63/323,358, filed on Mar. 24, 2022, the contents of which is hereby incorporated by reference in its entirety for all purposes.

### BACKGROUND OF THE DISCLOSURE

Phosphatidylinositol 3-kinases (PI3Ks) comprise a family of lipid kinases that catalyze the transfer of phosphate to the 3-position of the inositol ring of phosphatidylinositol and its derivatives to produce phosphoinositol-3-phosphate (PI(3)P), phosphoinositol-3,4-diphosphate (PI(3,4)P2) and phosphoinositol-3,4,5-triphosphate (PI(3,4,5)P3) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). PI3Ks have been divided into three classes according to their structural characteristics and substrate specificity. Class IA PI3Ks are heterodimers composed of a p110 catalytic subunit and a p85 regulatory subunit. In mammals, there are three genes, PIK3CA, PIK3CB and PIK3CD, encoding p110 catalytic isoforms: p110α, p110β and p110δ, respectively. There are also three genes, PIK3R1, PIK3R2 and PIK3R3, encoding p85a (and its splicing variants p55α and p50 α), p85β and p55γ regulatory subunits, respectively, collectively called p85. The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class IB PI3K is a heterodimer composed of a catalytic subunit p110γ and a regulatory subunit p101. p110γ is mainly expressed in leukocytes and can be activated directly by GPCRs. Class II PI3Ks are monomers with only a single catalytic subunit. Class III PI3Ks consists of a single catalytic subunit Vps34 (homolog of the yeast vacuolar protein-sorting defective 34). The phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)). There is this interest and a current need for binding to and regulating PI3K for treatment of various diseases.

KRAS mutations is prevalent in human cancers. Globally, among the about 18 million new cancer diagnoses each year, about 2.6 million cancer patients are estimated to harbor a KRAS mutation. It is estimated that KRAS mutations alone account for about 1 million annual deaths worldwide. Specifically, gain-of-function missense mutations in KRAS account for the majority of RAS gene mutations in human cancer (75%). See. e.g. Kwan, A. K., Piazza, G. A., Keeton, A. B. et al. The path to the clinic: a comprehensive review on direct KRASG12C inhibitors. J Exp Clin Cancer Res 41, 27 (2022), which is incorporated by reference herein.

More specifically, the KRASG12C mutation is present in about 12% of all KRAS-mutant cancers, yet there is only one approved drug in the United States that specifically inhibits the KRASG12C mutation. There is thus a need for additional therapeutics for treating KRAS, including specific mutations such as KRASG12C and in combination with other cancer therapeutics that bind to a different target.

### SUMMARY

The present invention includes pharmaceutical combinations or methods of use thereof of pharmaceutical combinations comprising a therapeutically effective amount of a compound that forms a covalent bond with a kinase or pseudokinase or a pharmaceutically acceptable salt thereof, and at least one additional therapeutically active agent. In one specific embodiment, the kinase is PI3-kinase (PI3K).

In another embodiment, the compound that forms a covalent bond with a kinase or pseudokinase is a compound having the formula:

(FCB)a-(L)b-(CLM)c,
or a pharmaceutically acceptable salt thereof, wherein:
CLM is a covalent linking modality;
L is a linker;
a and c are each independently integers between 1 and 5;
b is an integer between 0 and 5; and
FCB comprises:

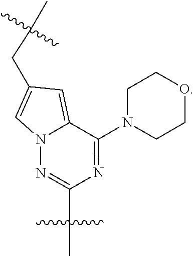 [SMILES: *Cc1cc2c(N3CCOCC3)nc(*)nn2c1]

In a specific embodiment, the FCB has the structure:

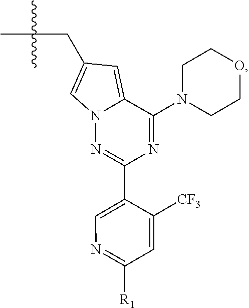

wherein R1 is selected from the group consisting of:

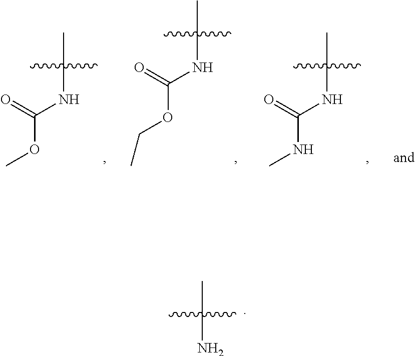

In another specific embodiment, the compound is Compound I:

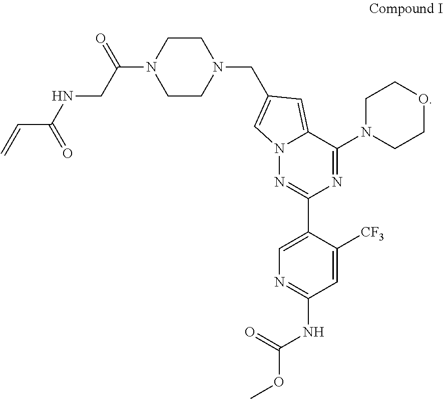 [SMILES: C=CC(=O)NCC(=O)N1CCN(Cc2cc3c(N4CCOCC4)nc(-c4cnc(NC(=O)OC)cc4C(F)(F)F)nn3c2)CC1]

Compound I is disclosed in WO 2021/055747, which is hereby incorporated by reference in its entirety. In some embodiments, Compound I is a phosphoinositide 3-kinase (PI3K) inhibitor. In some embodiments, Compound I is an irreversible inhibitor of PI3K. In embodiments, Compound I is an irreversible inhibitor of PI3Kα. In other embodiments, Compound I can form a covalent bond with an amino acid of a PI3K. In other specific embodiments, Compound I can form a covalent bond with a cysteine in a PI3K, e.g., PI3Kα (e.g., via a Michael reaction).

In another specific embodiment, the additional therapeutically active agent is a RAS inhibitor or preferably a KRAS inhibitor or an inhibitor of the KRAS pathway, such as in inhibitor of BRAF, MEK and/or ERK or an inhibitor of the RAS/RAF/MEK/MAPK signaling pathway. In another embodiment, the additional therapeutically active agent is an inhibitor of a KRAS mutant. In a specific embodiment, the additional therapeutically active agent is specific to the KRAS mutant G12C. In another specific embodiment, the additional therapeutically active agent covalently binds to KRAS-G12C.

In another specific embodiment, the additional therapeutically active agent is a KRAS inhibitor selected from one or more of the group consisting of sotorasib, adagrasib, ARS-3248, LY3499446, LY3537982, GDC-6036, D-1553, JDQ443, and BI 1823911. In another specific embodiment, the KRAS inhibitor is selected from one or more of the group consisting of sotorasib and adagrasib.

In another embodiment, the KRAS inhibitor is adagrasib. In a specific embodiment, the dosage amount of adagrasib is the range of about 200 mg to about 2000 mg, or about 400 mg to about 1000 mg, or about 500 mg to about 700 mg. In another embodiment, the dose is administered once, twice, or three times a day. In one embodiment, the at least one additional therapeutically active agent is a MEK inhibitor, such as a MEK1 and/or MEK2 inhibitor. In another embodiment, the MEK inhibitor or inhibitor of the RAS/RAF/MEK/MAPK signaling pathway, is MEK162, XL518, Refametinib, Pimasertib, selumetinib, GSK1120212, PD-325901, E6201, GDC-0623, CH5126766 AZD-6244, CL-1040 and/or TAK-733. In another embodiment, the at least one additional therapeutically active agent is a BRAF inhibitor. In a specific embodiment, the BRAF inhibitor is Sorafenib, Vemurafenib (Zelboraf), dabrafenib (Tafinlar), and/or encorafenib (Braftovi). In another embodiment, the at least one additional therapeutically active agent is a ERK inhibitor. In a specific embodiment, the ERK inhibitor is CC-90003, GDC-0994, DEL-22379, XMD8-92, ADTL-EI1712, MK-8353, BVD-523, LY3214996, KO-947, ONC201, AZD0364, HH2710, and/or LTT462.

In another embodiment, the at least one additional therapeutically active agent is a PI3K/AKT inhibitor or a PI3K/AKT pathway inhibitor. Dysregulation of the PI3K/Akt pathway is frequently observed in human cancers. PI3K activation leads to the Akt activation and subsequent activation of downstream effectors such as mTOR and others may be associated with resistance to both chemotherapeutic agents and target agents. Accordingly, the at least one additional therapeutically active agent is a PI3K/AKT inhibitor or a PI3K/AKT pathway inhibitor, i.e., an inhibitor in the pathway such as inhibiting a downstream effector such as RHEB or mTOR, including mTORC1 and/or MTORC2 inhibitors. In some embodiments, the PI3K/Akt inhibitor is selected from the group consisting of SC66, MK-2206, AZD5363, AZD8835, AZD8186, BKM120, XL147, GDC0941, GSK1059615, PX-866, CAL-101, KRX0401, VQD-002, XL418, PX316, BYL719, BVD-523, PA-79, ZSTK474, BAY80-6946, B591, TG-100-115, RIDR-PI-103, TAK-117, GSK2636771, CAL-101, PWT-143, AMG319, YY-20394, IBI-376, TGR-1202, RP6530, GDC-0032, IPI-145, CDZ173, IPI-549, BEZ-235, GDC-0980, PF-05212384, SF1126, GSK458, LY3023414, PQR309, GDC0084, XL765, ARQ092, BAY1125976, TAS117, GSK2110183, GDC0068, GSK2141795, GSK690693, and/or PF-04691502.

In some embodiments, the pharmaceutical combinations and/or compositions of the present disclosure further comprise as an at least one additional therapeutically active agent a PI3K/Akt downstream mTOR pathway inhibitor such as zotarolimus, umirolimus, temsirolimus, sirolimus, sirolimus NanoCrystal, everolimus, biolimus A9, ridaforolimus, rapamycin, TCD-10023, DE-109, MS R001, MS R002, MS-R003, (−)-rapamycin, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, and/or ABTL-0812. In some embodiments, the pharmaceutical combinations and/or compositions of present disclosure further comprise as an at least one additional therapeutically active agent is a Rheb inhibitor. In a specific embodiment, the Rheb inhibitor is HY-124798.

In one embodiment, the compound and one or more active agents of the pharmaceutical combinations are administered together. In a specific embodiment, the compound and one or more active agents are in the same dosage form. In another embodiment, the compound and one or more active agents are in different dosage forms but administered at the same time. In another embodiment, the compound and one or more active agents are in different dosage forms and one is administered to a subject before the other dosage form.

The present invention also comprises the administration of the pharmaceutical combinations described herein to a subject. In other specific embodiments, the methods may include treating or inhibiting cancer in a subject. In a another specific embodiment, the methods may include treating or ameliorating a cell proliferation disorder in a subject, the method comprising administering to a subject in need thereof a therapeutically effective amount of a compound that forms a covalent bond with a kinase or pseudokinase or a pharmaceutically acceptable salt thereof, and administering at least one additional therapeutically active agent before, during, or after the subject has been administered a compound that forms a covalent bond with a kinase or pseudokinase. In a specific embodiment, the kinase is PI3-kinase (PI3K), and preferably PI3Kα.

In another embodiment of treating or ameliorating a cell proliferation disorder in a subject, the cell proliferation disorder is cancer. In a specific embodiment, the cancer is selected from the group consisting of: heme cancer, colorectal cancer, ovarian cancer, breast cancer, cervical cancer, lung cancer, liver cancer, colon cancer, pancreatic cancer, cancer of the lymph nodes, colon cancer, small intestine, prostate cancer, brain cancer, cholangiocarcinoma, gallbladder carcinoma, cancer of the head and neck, bone cancer, Ewing's sarcoma, skin cancer, kidney cancer, and cancer of the heart. In another embodiment, the cancer is selected from the group consisting of breast cancer, ovarian cancer, pancreatic cancer, colorectal cancer, colon cancer, small intestine and lung cancer. In another embodiment of the present invention, the subject is human.

### DETAILED DESCRIPTIONS OF THE DISCLOSURE

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; for example, “a PI3-kinase (PI3K) modulator” refers to one or more PI3-kinase (PI3K) modulators or at least one PI3-kinase (PI3K) modulator. In embodiments, the PI3-kinase (PI3K) modulator is a PI3Ka modulator. As such, the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The terms “excipient”, “carrier”, and “vehicle” are used interchangeably throughout this application and denote a substance with which a compound of the present invention is administered.

The term “combination therapy” refers to a first therapy that includes a compound having the formula: (FCB)a-(L)b-(CLM)c, and/or their subgenera, or Compound I, or pharmaceutically acceptable salt thereof, in conjunction with a second therapy (e.g., therapy, surgery and/or an additional pharmaceutical agent) useful for treating, stabilizing, preventing, and/or delaying the disease or condition. Administration in “conjunction with” another therapeutically active agent includes administration in the same or different composition(s) and/or combinations, either sequentially, simultaneously, or continuously, through the same or different routes. In some embodiments, the combination therapy optionally includes one or more pharmaceutically acceptable carriers or excipients, non-pharmaceutically active compounds, and/or inert substances.

The terms “pharmaceutical combination,” “therapeutic combination” or “combination” as used herein, refers to a single dosage form comprising at least two therapeutically active agents, or separate dosage forms comprising at least two therapeutically active agents together or separately for use in combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration.

As used herein, the terms “additional pharmaceutical agent” or “additional therapeutic agent” or “additional therapeutically active agent” with respect to the compounds described herein refers to an active agent other than the compounds of and/or their subgenera, or Compound I, which is administered to elicit a therapeutic effect. The pharmaceutical agent(s) may be directed to a therapeutic effect related to the condition that the compounds of the present disclosure is intended to treat or prevent (e.g., cancer) or to further reduce the appearance or severity of side effects of the compounds of the present disclosure.

As used herein, the phrase “a disorder characterized by cell proliferation” or “a condition characterized by cell proliferation” include, but are not limited to, cancer, benign and malignant tumors. Examples of cancer and tumors include, but are not limited to, cancers or tumor growth of the colorectum, breast, lung, liver, pancreas, lymph node, colon, prostate, brain, head and neck, skin, kidney, blood and heart (e.g., leukemia, lymphoma, carcinoma).

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods, compositions, and combinations of the present application.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

“Therapeutically effective amount” means the amount of a compound or a therapeutically active agent that, when administered to a patient for treating a disease or other undesirable medical condition, is sufficient to have a beneficial effect with respect to that disease or condition. The therapeutically effective amount will vary depending on the type of the selected compound or a therapeutically active agent, the disease or condition and its severity, and the age, weight, etc. of the patient to be treated. Determining the therapeutically effective amount of a given compound or a therapeutically active agent is within the ordinary skill of the art and requires no more than routine experimentation.

The term “therapeutically effective” applied to dose or amount refers to that quantity of a compound or pharmaceutical formulation that is sufficient to result in a desired clinical benefit after administration to a patient in need thereof.

As used herein, a “subject” can be a human, non-human primate, mammal, rat, mouse, cow, horse, pig, sheep, goat, dog, cat and the like. In embodiments, the subject is human. In embodiments, the subject can be suspected of having or at risk for having a cancer. The terms “subject” and “patient” are used interchangeably throughout the present application.

“Mammal” includes humans and both domestic animals such as laboratory animals (e.g., mice, rats, monkeys, dogs, etc.) and household pets (e.g., cats, dogs, swine, cattle, sheep, goats, horses, rabbits), and non-domestic animals such as wildlife and the like.

All weight percentages (i.e., “% by weight” and “wt. %” and w/w) referenced herein, unless otherwise indicated, are measured relative to the total weight of the pharmaceutical composition.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

Compounds that Form a Covalent Bond with PI3-Kinase (PI3K)

The compounds, such as those compounds having the formula:

(FCB)a-(L)b-(CLM)c,

or a pharmaceutically acceptable salt thereof, and/or their subgenera, or Compound I, or salts, solvates, esters and/or prodrugs thereof disclosed herein, can be used in a combination with at least one additional therapeutically active agent or therapy (combination therapy).

The PI3K compounds include for example, Anchored Relational Covalent System, hereafter referred to as the ARCS, which comprises a Functionally Competent Binder, hereafter referred to as the FCB; a Covalent Linking Modality, hereafter referred to as the CLM, wherein the CLM is attached directly or indirectly to said therapeutic modality; and optionally a linker positioned between the FCB and the CLM. In some embodiments, a CLM is covalently attached to an FCB directly with a bond. In some embodiments, a CLM is covalently attached to an FCB indirectly with a linker.

The term “ARCS” as used herein, refers to any therapeutic conjugate that is formed by linking an FCB and a CLM with a bond or a linker. In some embodiments, the ARCS can form a covalent bond with one or multiple targets such as nucleotides, oligonucleotides, peptides, or proteins. In some embodiments, the ARCS can form a covalent bond with a biological target. The covalent bond can be detected with any known method in the art. As a non-limiting example, covalent attachment of azido-small molecules to the proteins can be detected by using click chemistry to attach heavy, PEG-containing alkynes to the small molecules. The covalently labeled proteins are detected by a gel shift that occurs because they are now PEG-labeled and have a higher molecular weight (Biochemistry 2018, 57:5769-5774). In another non-limiting example, mass spectrometry can be used to detect covalently-labeled, purified protein (Nature Chemical Biology 2007, 3:229-238). In yet another non-limiting example, cellular quantitative mass spectrometry-based proteomic methods can be used to analyze covalent bonding (Cell Chemical Biology 2017, 24:1388-1400.e7). In yet another non-limiting example, X-ray crystallography is used to confirm covalent bond formation (Nature Chemical Biology 2007, 3:229-238; J. Med. Chem. 2020, 63:52-65). In yet another non-limiting example, mass spectrometry of in-cell, covalently-labeled, and affinity-enriched samples can be used to reveal the site of covalent modification (Nat. Chem. Biol. 2016, 12:876-884).

In some embodiments, the ARCS can form a covalent bond with the biological target to a percent of about 5%-100% of the biological target. In some embodiments, the ARCS can form a covalent bond with the biological target from about 5%, 10%, 15%, 20%, 25%, 30%, 35% 40%, 45%, 50%, 55%, 60%, 65%, 70%, 75%, 80%, 85%, 90%, 95% or 100%. In some embodiments, the covalent bond is formed in an aqueous solution at a temperature of 0-50° C., within 48 hours, and at a treatment dose of 10 mM.

Not willing to be bound to any theory, the ARCS may first form a non-covalent bond with a biological target (such as a target protein) via an FCB, and then form a covalent bond with the biological target via a CLM. In some embodiments, the efficacy of the ARCS is better than the efficacy of the FCB alone. In some embodiments, the CLM does not substantially interfere with efficacy of the FCB. In some embodiments, the FCB does not substantially interfere with covalent binding of the CLM. In some embodiments, the toxicity of the ARCS is less than the toxicity of the FCB alone. The term “FCB” as used herein, refers to a therapeutic modality that can be a known drug, a diagnostic compound, a drug candidate and a functional fragment and/or combination of any of the forgoing. The FCB encompasses free acid and free base forms; optical and tautomeric isomers; isotopes including radioisotopes and pharmaceutically acceptable salts of the drug, prodrug or fragment thereof. The FCBs may be small molecules, proteins, peptides, lipids, carbohydrates, sugars, nucleic acids, or combination thereof. In some embodiments, the FCBs are nucleic acids including, but is not limited to DNA or RNA. The FCB may be a therapeutic agent such as, but not limited to, anticancer agents, anti-neurodegenerative agents, autoimmune drugs and anti-aging agents. The FCB may bind to a biological target non-covalently. In some embodiments, the FCB may be a functional fragment of a drug. The term “functional fragment” as used herein, refers to a part of a drug or derivative or analog thereof that is capable of inducing a desired effect of the drug. In some embodiments, the FCB may comprise an alkyne functional group. In some embodiments, the FCB may not comprise an alkyne functional group.

As used herein, the term “peptide”, “polypeptide”, “protein” refers to a polymer composed of amino acid monomers linked by an amide bond. Amino acids may be D- or L-optical isomer. Peptides may be formed by condensation or coupling reaction with the amino group of one α-carbon carboxyl group and another amino acid. Peptides may be non-linear branched peptides or cyclic peptides. Furthermore, the peptide may be optionally modified or protected with divergent functional group or a protecting group including amino and/or carboxy termini.

Amino acid residues of the peptide are abbreviated as follows. Phenylalanine is Phe or F, leucine is Leu or L, isoleucine is Ile or I, methionine is Met or M, valine is Val or V, serine is Ser or S, proline is Pro or P, threonine is Thr or T, alanine is Ala or a, tyrosine is Tyr or Y, histidine is His or H, glutamine is Gln or Q, asparagine Asn or is N, lysine is Lys or K, aspartic acid is Asp or D, glutamic acid is Glu or E, cysteine is Cys or C, tryptophan is Trp or W, arginine is Arg or R, and glycine is Gly or G.

The term “CLM” as used herein, refers to any covalent binding modality that is capable of forming a covalent bond with the biological target. The CLM may be linked to an FCB by a bond or by a linker. The CLM may comprise one or more chemical moieties which can form a covalent bond with the biological target. The chemical moieties may be an electrophilic or nucleophilic group.

The CLM may be a small molecule having a molecular weight of less than about 1,000 Da, less than about 900 Da, less than about 800 Da, less than about 700 Da, less than about 600 Da or less than about 500 Da. In some cases, the CLM may have a molecular weight of between about 5 Da and about 1,000 Da, between about 10 Da and about 900 Da, in some embodiments between about 20 Da and about 700 Da, in some embodiments bout 20 Da and about 500 Da, between about 50 Da and about 400 Da, in some embodiments between about 100 Da and about 300 Da, and in some embodiments between about 150 Da and about 300 Da. The molecular weight of the CLM may be calculated as the sum of the atomic weight of each atom in the formula of the CLM multiplied by the number of each atom. It may also be measured by mass spectrometry, NMR, chromatography, light scattering, viscosity, and/or any other methods known in the art. It is known in the art that the unit of molecular weight may be g/mol, Dalton (Da), or atomic mass unit (amu), wherein 1 g/mol=1 Da=1 amu.

The term “biological target”, as used herein, refers to any target to which an FCB binds non-covalently to product a therapeutic effect. A CLM binds to the biological target covalently. In some embodiments, the biological target is a protein. Non-limiting examples of biological targets include kinase such as, but not limited to phosphoinositide 3-kinases (PI3Ks) and pseudokinase.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. Throughout the application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S— alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

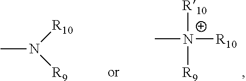

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

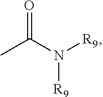

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

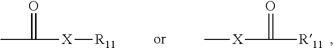

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analog of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The terms “polypeptide,” “peptide” and “protein” generally refer to a polymer of amino acid residues. As used herein, the term also applies to amino acid polymers in which one or more amino acids are chemical analogs or modified derivatives of corresponding naturally occurring amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

The present disclosure relates to a phosphoinositide 3-kinase (PI3K) inhibitor, such as Compound I, or a pharmaceutically acceptable salt thereof, solvate thereof, or salt solvate thereof.

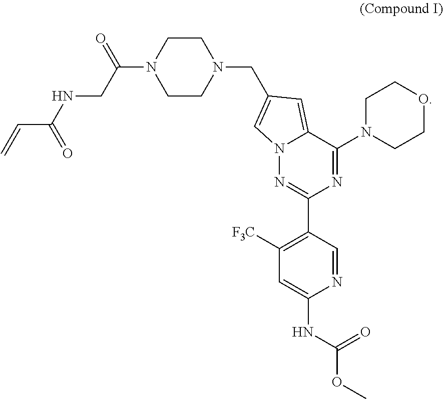 [SMILES: C=CC(=O)NCC(=O)N1CCN(Cc2cc3c(N4CCOCC4)nc(-c4cnc(NC(=O)OC)cc4C(F)(F)F)nn3c2)CC1]

The present invention also includes Compound I or a pharmaceutically acceptable salt thereof as the compound in any combination therapy or method of use or treatment with an additional therapeutically active agent.

### CLM

The ARCS of the present disclosure contains one or more CLM(s). The CLM can be any covalent binding modality that is capable of forming a covalent bond with a biological target. The CLM may comprise one or more chemical moieties, one or more of which are capable of forming a covalent bond with a biological target. In certain embodiments, the CLM may comprise an internal linker or spacer. The internal linker or spacer may combine two parts of the CLM or can be joined to the CLM.

In some embodiments, the CLM is a small molecule. In some embodiments, the CLM has a molecular weight of less than about 1000 Dalton (e.g., less than about 900, 800, 750, 700, 650, 600, 550, 500, 450, 400, 350, 300, 250, 200, 150, 100, etc.).

In certain embodiments, the CLM of the ARCS comprises a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the ARCS is 100%. The amount of CLM(s) of the ARCS may also be expressed in terms of proportion to the FCB(s). For example, the present teachings provide a ratio of FCB to CLM of about 10:1, 9:1, 8:1, 7:1, 6:1, 5:1, 4:1, 3:1, 2:1, 1:1, 1:2,1:3, 1:4; 1:5, 1:6, 1:7, 1:8, 1:9, or 1:10.

In some embodiments, the CLM comprises at least one substituted or unsubstituted alkyne. In some embodiments, the CLM comprises at least one substituted or unsubstituted acrylamide. In some embodiments, the CLM comprises at least one substituted or unsubstituted vinyl sulfonamide. In some embodiments, the CLM comprises at least one substituted or unsubstituted vinyl sulfone. In some embodiments, the CLM comprises at least one substituted or unsubstituted fumaramide. In some embodiments, the CLM comprises at least one substituted or unsubstituted acrylate. In some embodiments, the CLM comprises at least one substituted or unsubstituted isothiocyanate. In some embodiments, the CLM comprises at least one substituted or unsubstituted sulfonyl fluoride. In some embodiments, the CLM comprises at least one substituted or unsubstituted fluorosulfate. In some embodiments, the CLM comprises at least one substituted or unsubstituted formyl phenyl boronic acid. In some embodiments, the CLM comprises at least one substituted or unsubstituted boronic acid. In some embodiments, the CLM comprises at least one activated ester. In some embodiments, the CLM comprises at least one substituted or unsubstituted thioester. In some embodiments, the CLM comprises at least one sulfonyl group. In some embodiments, the CLM comprises at least one nitro group. In some embodiments, the CLM comprises at least one substituted or unsubstituted epoxide. In some embodiments, the CLM comprises at least one substituted or unsubstituted formyl phenyl boronic acid. In some embodiments, the CLM comprises at least one substituted or unsubstituted aryl halide. In some embodiments, the CLM comprises at least one substituted or unsubstituted aldehyde. In some embodiments, the CLM comprises at least one substituted or unsubstituted triazine. In some embodiments, the CLM comprises at least one substituted or unsubstituted cyano-acrylamide. In some embodiments, the CLM comprises at least one substituted or unsubstituted chloroacetamide.

Exemplary CLMs include, but not limited to

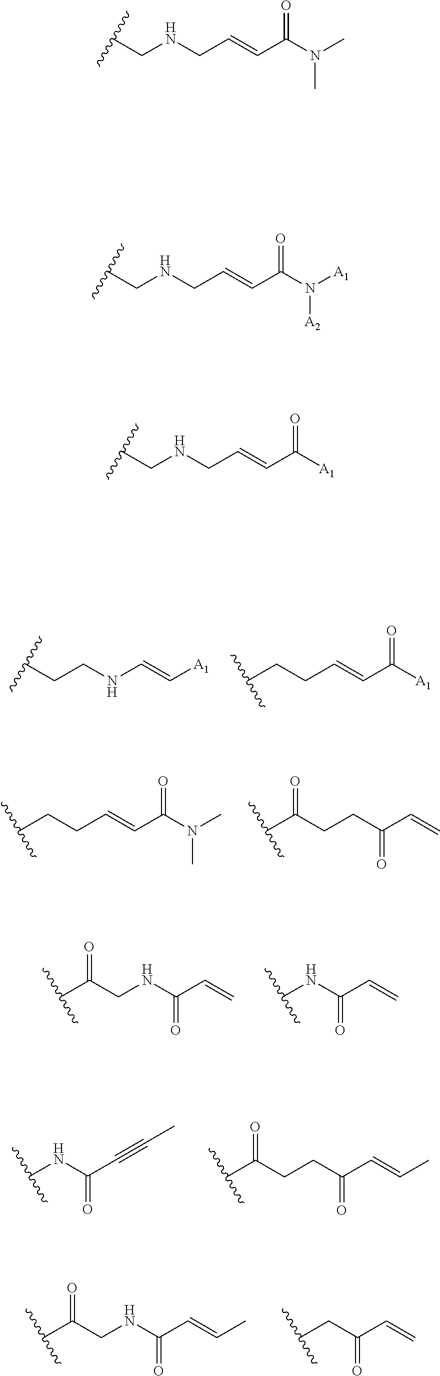

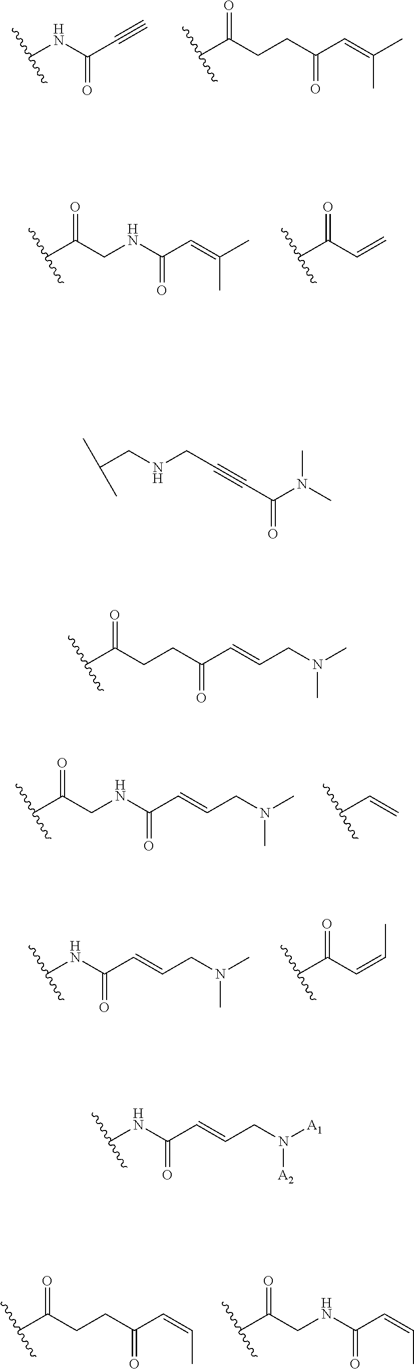

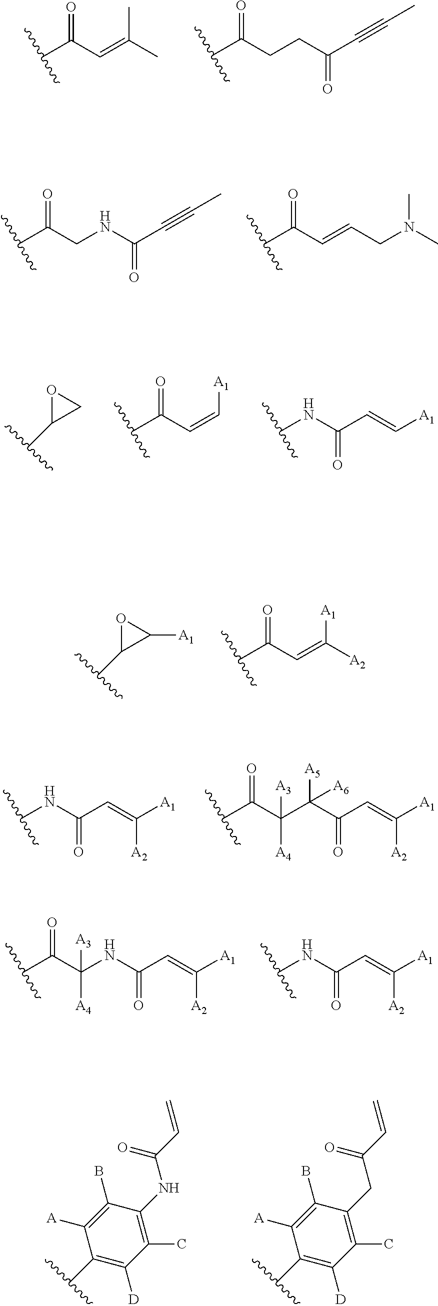

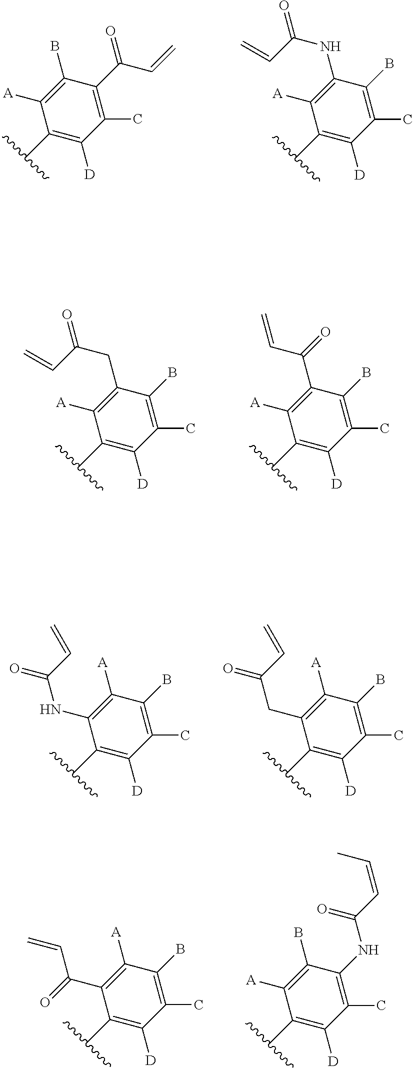

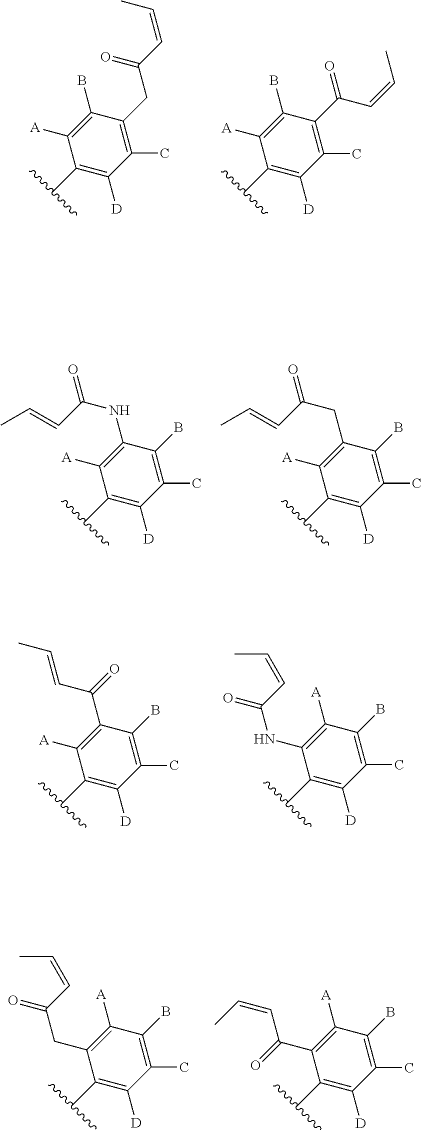

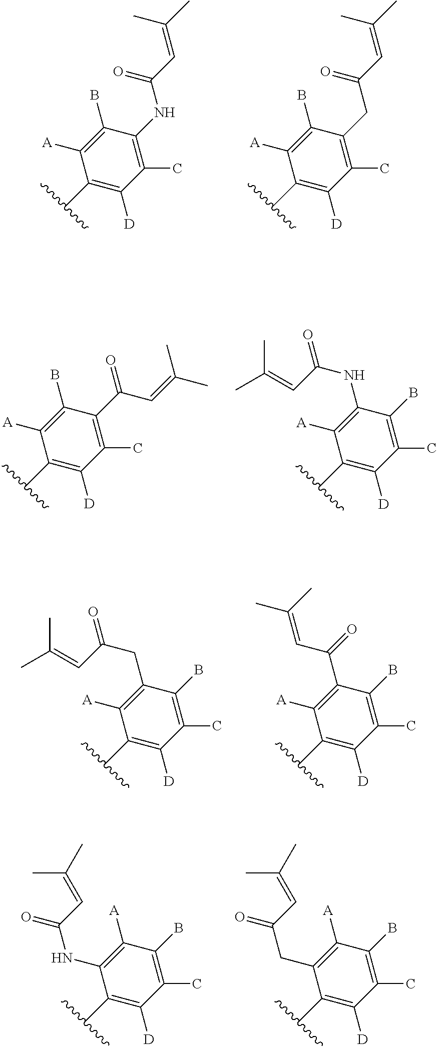

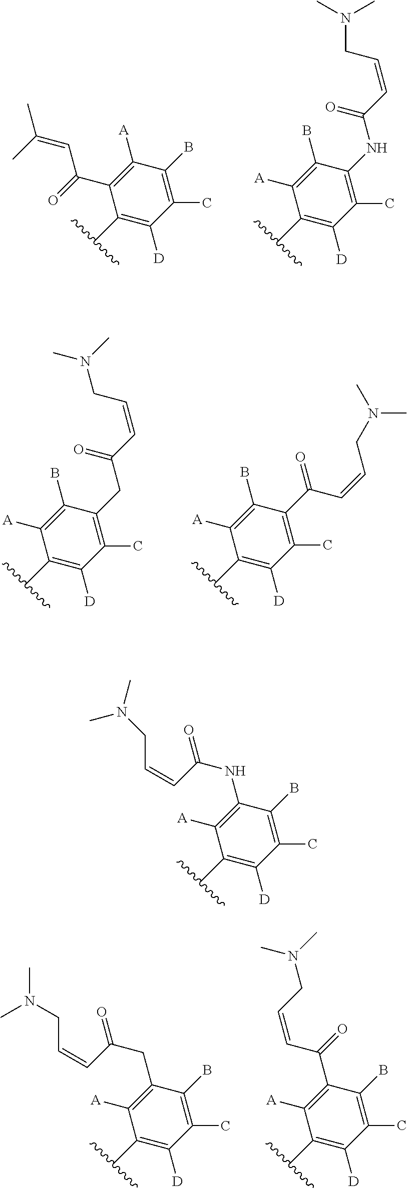

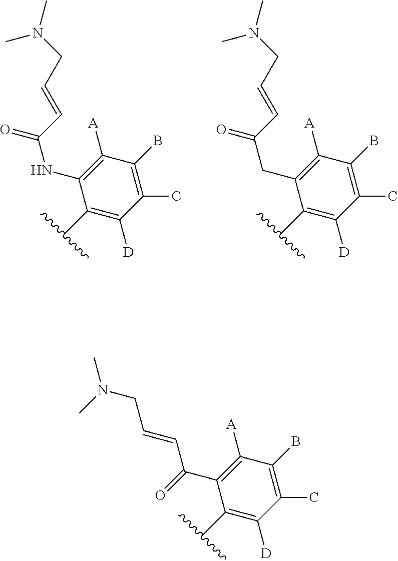

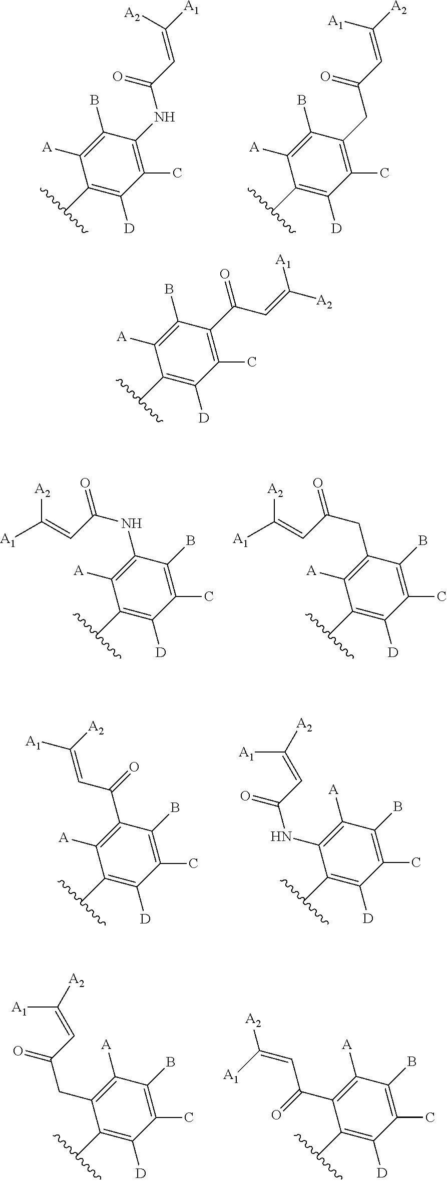

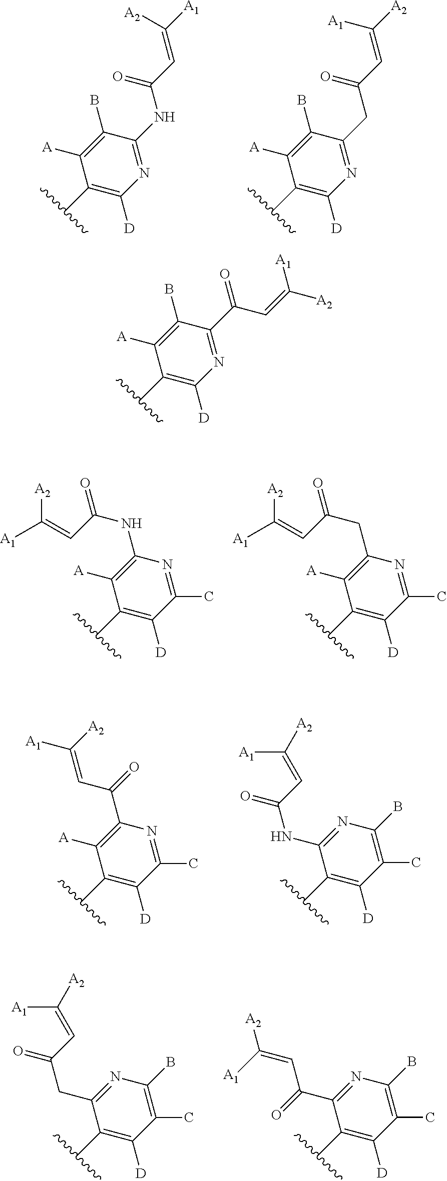

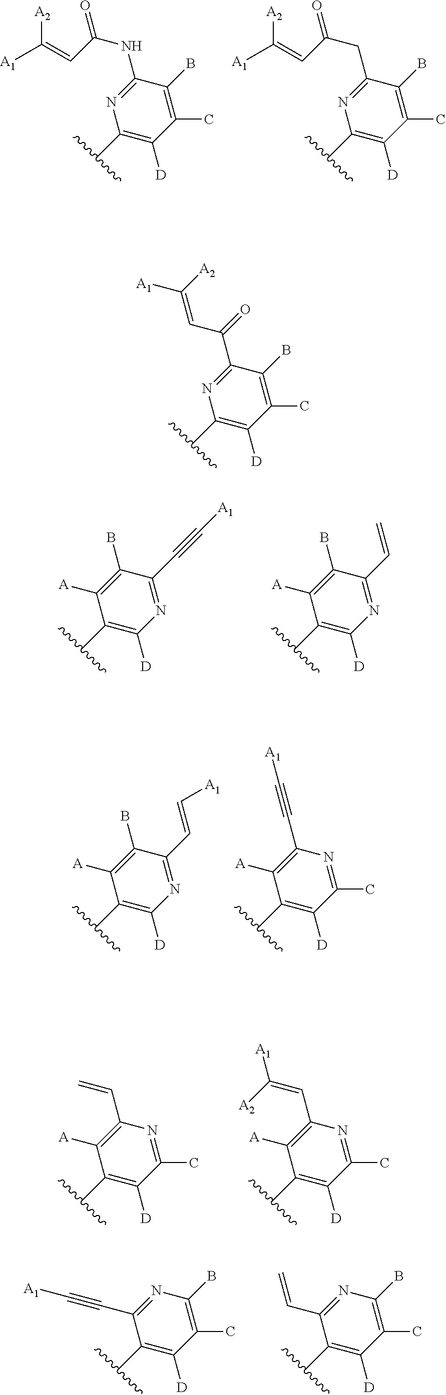

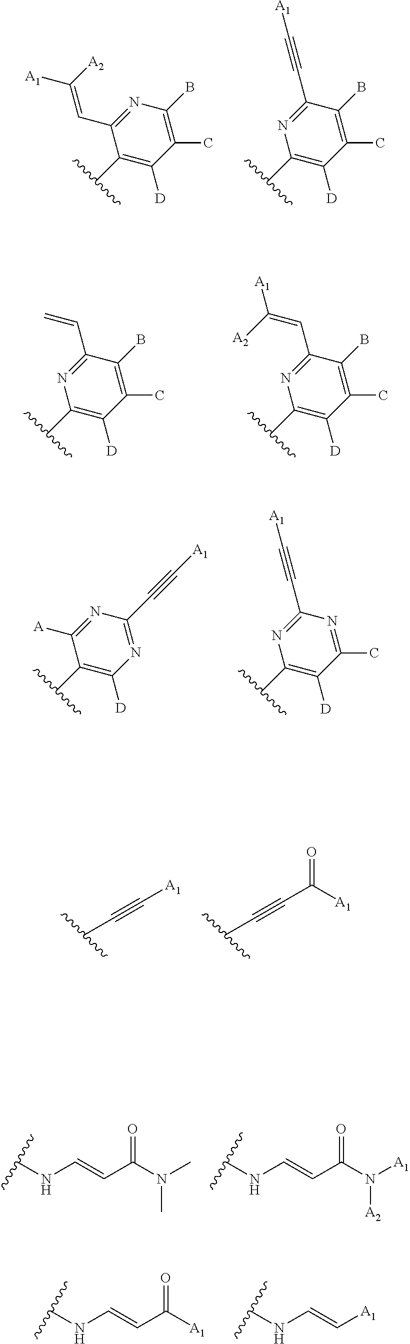

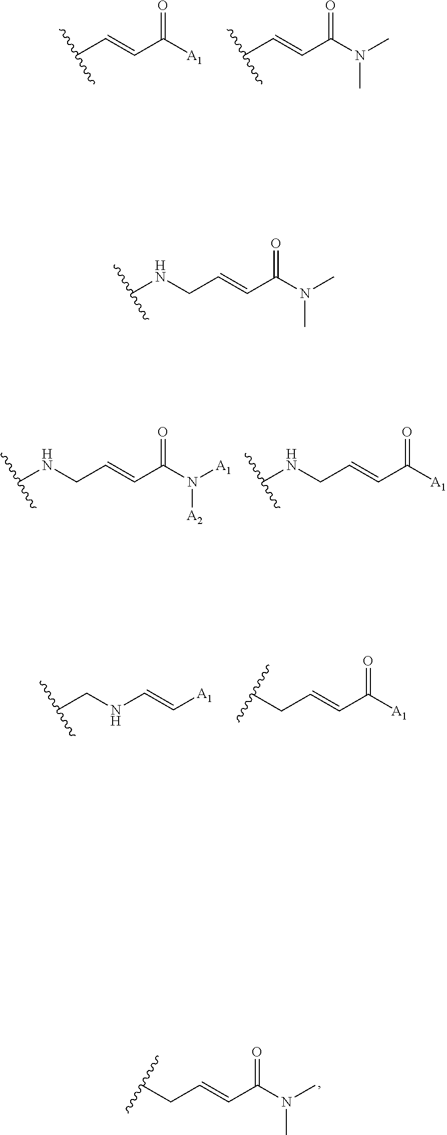

wherein A, B, C and D at each occurrence is independently selected from the group consisting of H, halogen, CF₃, —OH, —NH₂, —SH, —SCH₃, —CN, —NO₂, —CH₂(NH₂), —C(O)OH, —S(O)₂NH₂, —C(O)NH₂, —C(O)CH₃, NHC(O)—C₁₋₆ alkyl, N(C₁₋₃ alkyl)C(O)—C₁₋₆ alkyl, OC(O)NH₂, OC(O)NH(CH₃), OC(O)N(CH₃)₂, imidazolyl, pyrazolyl, methylimidazolyl, methylpyrazolyl, optionally substituted C₁₋₆ alkyl, optionally substituted C₂₋₆ alkenyl, optionally substituted C₂₋₆ alkynyl, optionally substituted C₃₋₆ cycloalkyl, optionally substituted 5-10 membered heterocycle, optionally substituted aryl, and optionally substituted 5-10 membered heteroaryl, wherein the optional substituents for A, B, C, and D are 1-3 substituents which are independently selected from the group consisting of halogen, OH, NH₂, CH₃, CF₃, —CN, —NO₂, —C(O)OH, —S(O)₂NH₂, —C(O)NH₂, —CH₂NH₂, —C(O)CH₃, SH, —S—CH₃, optionally substituted C₁₋₃ alkyl, and optionally substituted C₃₋₆ cycloalkyl.

A1, A2, A3, A4, A5, and A6 at each occurrence are independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for A1, A2, A3, A4, A5, and A6 are 1-3 substituents which are independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl, and
wherein the C1-3 alkyl and C3-6 cycloalkyl optional substituents are 1-2 substituents, which are independently selected from the group consisting of halogen, OH, NH2, CH₃, CF₃, —CN, —NO₂, —C(O)OH, —S(O)₂NH₂, —C(O)NH₂, —CH₂NH₂, —C(O)CH₃, SH, and —S—CH₃, or a fragment, derivative or analog thereof.

In some embodiments, the CLM is selected from the group consisting of

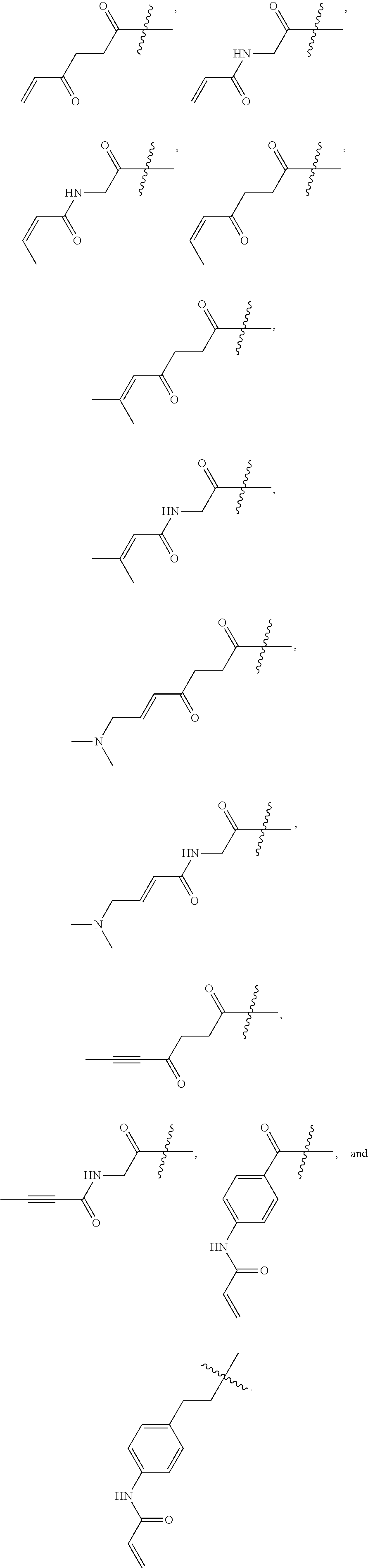

### Linkers

The ARCS of the present disclosure contains one or more optional linkers connecting the FCB(s) and CLM(s). The linker, L, can be attached to anywhere on FCB and CLM, as long as the efficacy of FCB and the binding of CLM are not significantly affected. In some embodiments, CLM comprises an optional internal linker.

In some embodiments, the linker (including the internal linker of CLM) is a small molecule. In some embodiments, the linker (including the internal linker of CLM) is selected, but not limited to substituted and unsubstituted C1-C30 alkyl, substituted and unsubstituted C2-C30 alkenyl, substituted and unsubstituted C2-C30 alkynyl, substituted and unsubstituted C3-C30 cycloalkyl, substituted and unsubstituted C1-C30 heterocycloalkyl, substituted and unsubstituted C3-C30 cycloalkenyl, substituted and unsubstituted C1-C30 heterocycloalkenyl, substituted and unsubstituted aryl, and substituted and unsubstituted heteroaryl.

In some embodiments, the linker (including the internal linker of CLM) can be a C1-C10 straight chain alkyl, C1-C10 straight chain O-alkyl, C1-C10 straight chain substituted alkyl, C1-C10 straight chain substituted O-alkyl, C4-C13 branched chain alkyl, C4-C13 branched chain O-alkyl, C2-C12 straight chain alkenyl, C2-C12 straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bipyridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments the linker contains one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the linker may be non-cleavable. In some embodiments, the linker may be cleavable. In some embodiments, the linker may be cleaved by an enzyme.

Non-limiting examples of linkers include

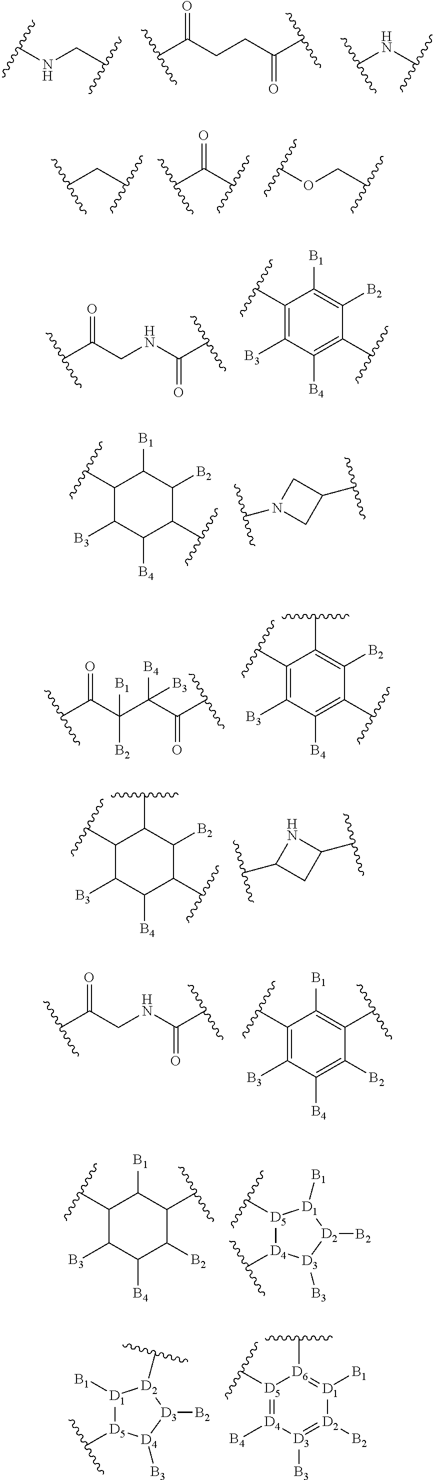

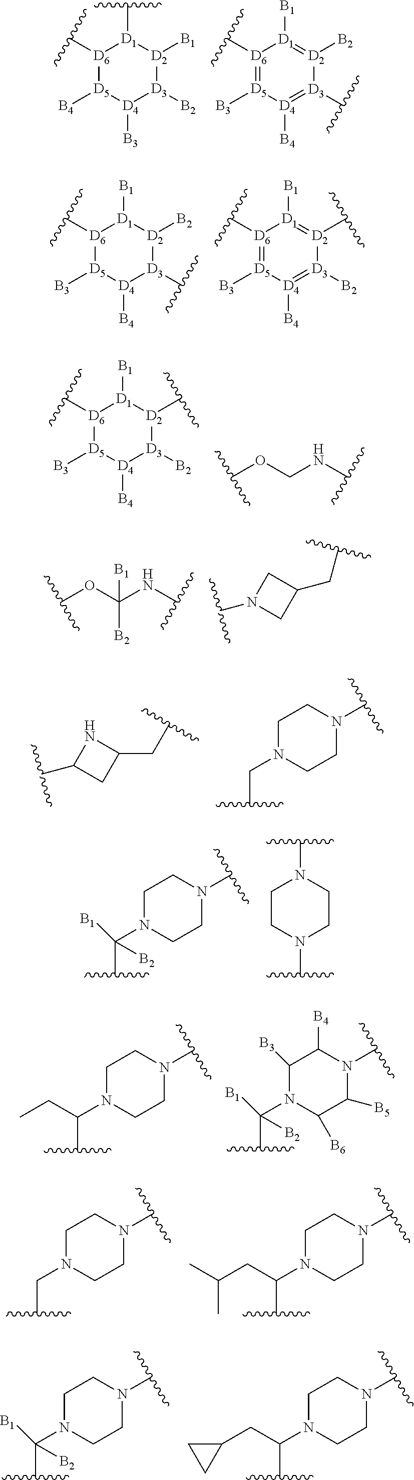

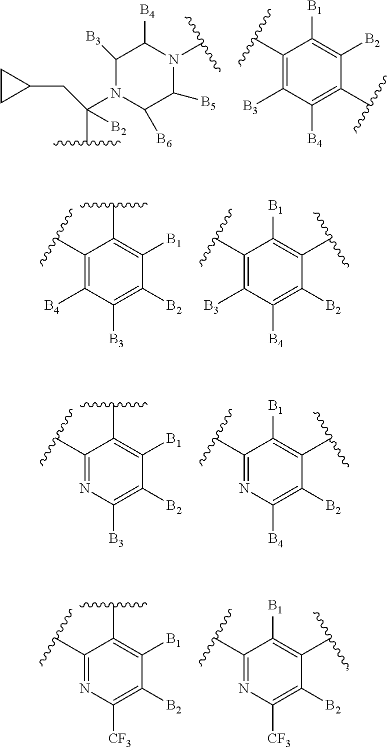

wherein D1, D2, D3, D4, D5 and D6 at each occurrence are independently selected from the group consisting of N, C, O, or S, provided that if D1-6 is a N, the corresponding position is trivalent; if D1-6 is a O or S, the corresponding position is divalent; wherein B1, B2, B3, B4, B5, and B6 at each occurrence is absent or independently selected from the group consisting of H, halogen, CF3, —OH, —CH3, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl, wherein the optional substituents for B1, B2, B3, and B4 are 1-3 substituents indpendently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl, and wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3, or any fragments or analogs thereof.

In some embodiments, the linker is selected from the group consisting of

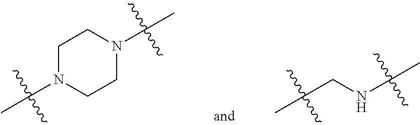

wherein either end can be connected to the CLM. In some embodiments, the linker selected from the group consisting of

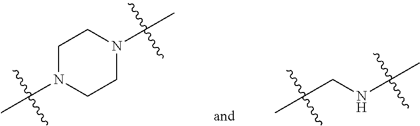

is connected to the CLM selected from the group consisting of

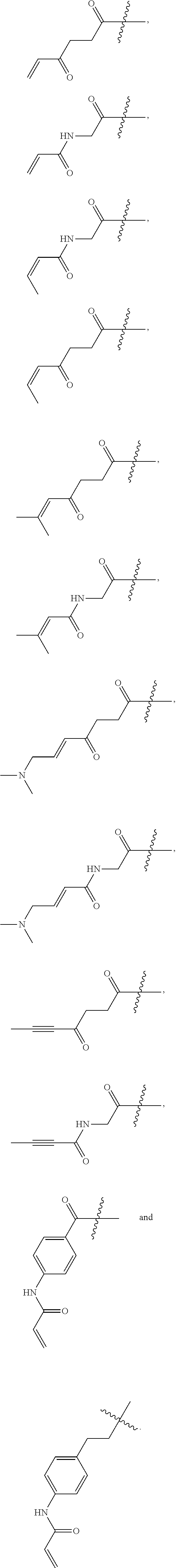

### ARCS

The ARCS of the present disclosure represents a class of drugs that have many advantages, such as an increased potency and extended duration of action, when compared to the reversible inhibitors. The present disclosure provides therapeutic conjugates that form a covalent bond with a kinase or pseudokinase. In some embodiments, the kinase is PI3-kinase (PI3K). The therapeutic conjugate may have a structure of

(FCB)a-(L)b-(CLM)c,

wherein a and c are, independently, integers between 1 and 5, b is an integer between 0 and 5, and wherein the FCB moiety comprises a PI3K inhibitor, or a fragment, analog or derivative thereof.

The FCB moiety, L (linker) moiety, and CLM moieties are discussed in the sections above. In one non-limiting example, the FCB comprises

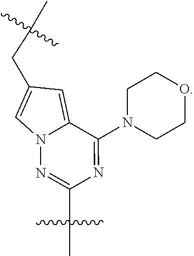 [SMILES: *Cc1cc2c(N3CCOCC3)nc(*)nn2c1]

In some embodiments, the FCB is a compound having the structure,

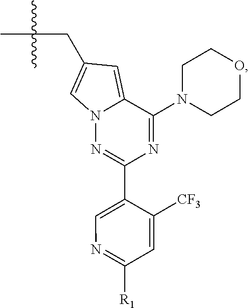

wherein R1 is selected from the group consisting of

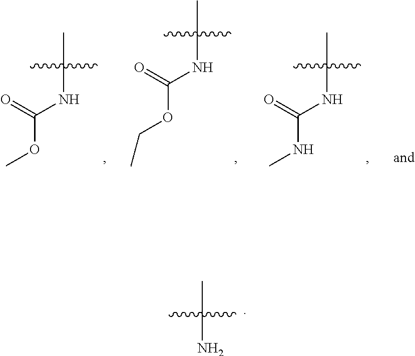

In some embodiments, the FCB is a compound having the structure,

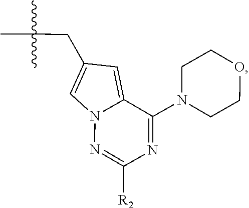

wherein R2 is selected from the group consisting of

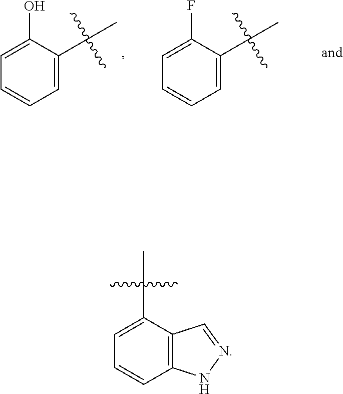 [SMILES: *c1ccccc1O ; *c1ccccc1F ; *c1cccc2[nH]ncc12]

In some embodiments, the linker is selected from the group consisting of

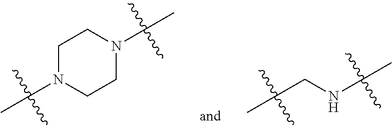

wherein either end can be connected to the CLM. In some embodiments, the CLM is selected from the group consisting of

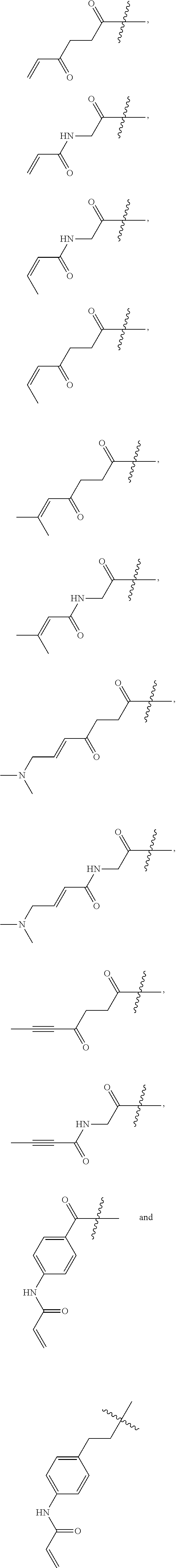

In some embodiments, the ARCS is selected from the group consisting of broad generic structures Compound 1-1 to Compound 1-5,

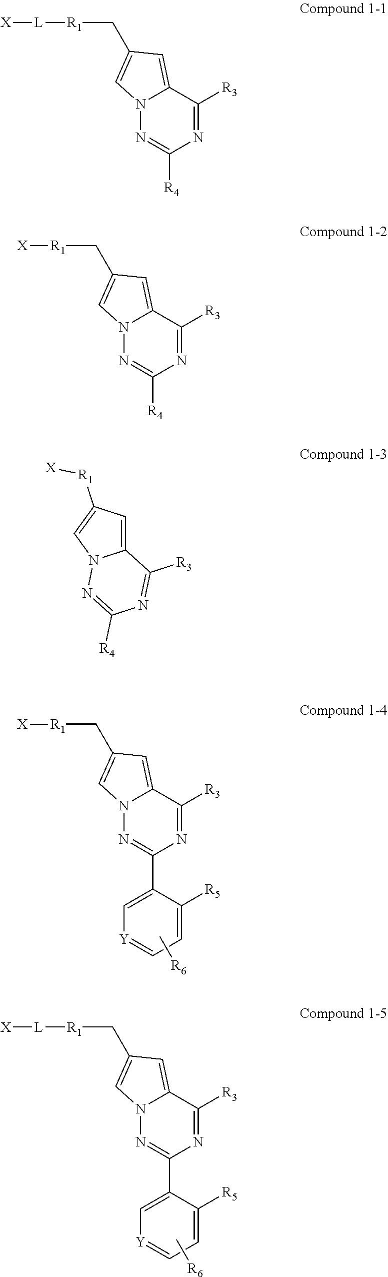

or a pharmaceutically acceptable salt thereof, wherein R1 at each occurrence is independently selected from the group

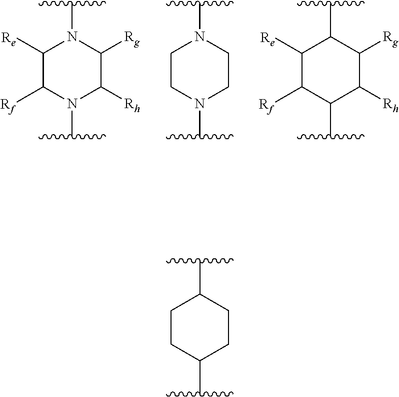

consisting of

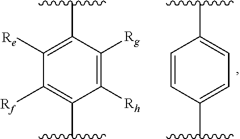

wherein R1 can be attached to X, L or the functional fragment of the drug in either of the two ends. For example, in

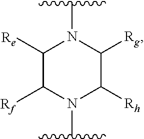

R1 can be attached to L either from the end adjacent to Re and Rg and to the functional fragment of the drug from the end adjacent to Rf and Rh; or R1 can be attached to L either from the end adjacent to Rf and Rh and to the functional fragment of the drug from the end adjacent to Re and Rg in Compound 1-1.

In some embodiments, R1 at each occurrence is independently selected from the group consisting of unsubstituted or substituted -(alk)a-S-(alk)b-, -(alk)a-O-(alk)b-, -(alk)a-NRA-(alk)b-, -(alk)a-C(O)-(alk)b-, -(alk)a-C(S)-(alk)b-, -(alk)a-S(O)-(alk)b-, -(alk)a-S(O)2-(alk)b-, -(alk)a-OC(O)-(alk)b-, -(alk)a-C(O)O-(alk)b-, -(alk)a-OC(S)-(alk)b-, -(alk)a-C(S)O-(alk)b-, -(alk)a-C(O)NRA-(alk)b-, -(alk)a-C(S)NRA-(alk)b-, -(alk)a-S(O)2NRA-(alk)b-, -(alk)a-NRAC(O)-(alk)b-, -(alk)a-NRAC(S)-(alk)b-, -(alk)a-NRAS(O)2-(alk)b-, -(alk)a-NRAC(O)O-(alk)b-, -(alk)a-NRAC(S)O-(alk)b-, -(alk)a-OC(O)NRA-(alk)b-, -(alk)a-OC(S)NRA-(alk)b-, -(alk)a-NRAC(O)NRB-(alk)b-, -(alk)a-NRAC(S)NRB-(alk)b-, and -(alk)a-NRAS(O)2NRB-(alk)b-;
a and b are independently selected from the group consisting of 0, 1, 2, 3, and 4;
alk is independently selected from the group consisting of C1-5 alkylene, C1-5 alkenylene, and C1-5 alkynylene, each of which is optionally substituted with 1-3 substituents independently selected from the group consisting of H, halogen, —OH, NH2, CF3, C1-5 alkyl, —CH2(NH2), —CN, —NO2, —C(O)OH, —S(O)2NH2, —C(O)NH2, —C(O)CH3, —SH, imidazolyl, pyrazolyl, methylimidazolyl, methylpyrazolyl, —O—C1-5 alkyl, —S—C1-5 alkyl, —NH—C1-5 alkyl, and —N(C1-5 alkyl)2, wherein the C1-5 alkyl groups are independently optionally substituted with 1-3 groups selected from the group consisting of halogen, —OH, —NH2, C1-4 alkyl, CF3, —CH2(NH2), —CN, —NO2, —C(O)OH, —S(O)2NH2, —C(O)NH2, —C(O)CH3, —SH, —SCH3, imidazolyl, pyrazolyl, methylimidazolyl, and methylpyrazolyl;
RA and RB, at each occurrence, are independently selected from the group consisting of hydrogen, C1-3 alkyl, C3-6 cycloalkyl, 5-10 membered heterocycle, aryl, and 5-10 membered heteroaryl, wherein the alkyl, cycloalkyl, heterocycle, aryl, and heteroaryl are each independently optionally substituted with 1-3 substituents selected from the group consisting of halogen, C1-3 alkyl, OH, NH2, NH—C1-3 alkyl, N(C1-3 alkyl)2, CF3, C1-6 alkyl, —CH2(NH2), —CN, —NO2, —C(O)OH, —S(O)2NH2, —C(O)NH2, —C(O)CH3, —SH, —SCH3, imidazolyl, pyrazolyl, methylimidazolyl, and methylpyrazolyl;
R3 at each occurrence is independently selected from the group consisting of:

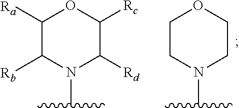

Ra, Rb, Rc, Rd, Re, Rf, Rg, and Rh at each occurrence are independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl;
wherein the optional substituents for Ra, Rb, Rc, Rd, Re, Rf, Rg, and Rh are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl, and
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

In some embodiments, R3 at each occurrence is independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for R3 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl, and wherein the C1-3 alkyl and C3-6 cycloalkyl optional substituents are 1-2 substituents independently selected from the group consisting of halogen, OH, NH₂, CH₃, CF₃, —CN, —NO₂, —C(O)OH, —S(O)₂NH₂, —C(O)NH₂, —CH₂NH₂, —C(O)CH₃, SH, and —S—CH₃.

R4 at each occurrence is independently selected from the group consisting of:

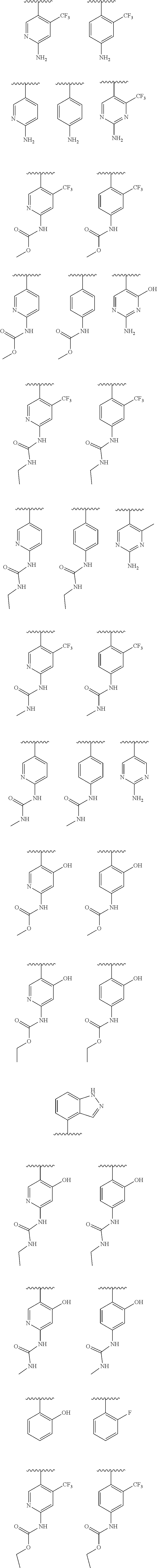

In some embodiments, R4 at each occurrence is independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for R4 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

R5 at each occurrence is independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for R5 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

R6 at each occurrence is independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for R6 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

L at each occurrence is independently selected from the group consisting of:

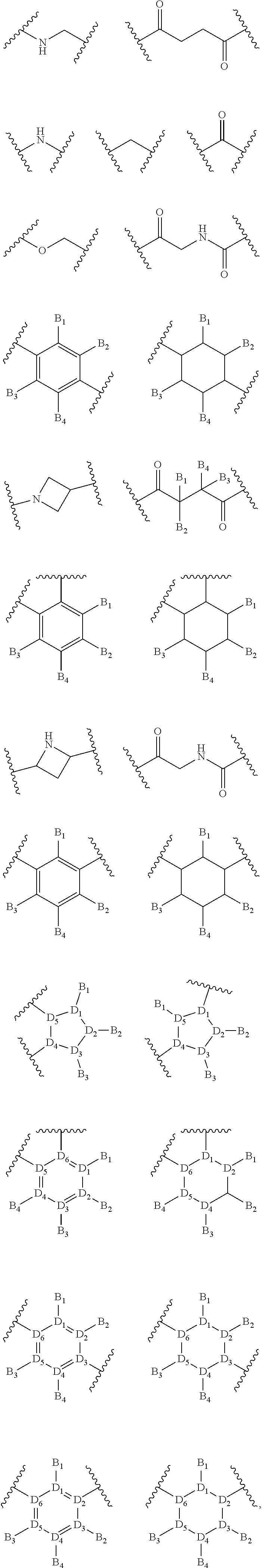

wherein D1, D2, D3, D4, D5, and D6 are at each occurrence are independently selected from the group consisting of N, C, O, or S, provided that if D1-6 is a N, the corresponding position is trivalent; if D1-6 is a O or S, the corresponding position is divalent. The linker can be attached to the CLM or the functional fragment of the drug on either of the two ends. For example, in

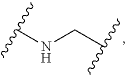 [SMILES: *CN*]

L can be attached to CLM either from the end adjacent to nitrogen or from the other end.

B1, B2, B3, and B4 are at each occurrence absent or independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for B1, B2, B3, and B4 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

In some embodiments, L at each occurrence is independently selected from the group consisting of unsubstituted or substituted -(alk)a-S-(alk)b-, -(alk)a-O-(alk)b-, -(alk)a-NRC-(alk)b-, -(alk)a-C(O)-(alk)b-, -(alk)a-C(S)-(alk)b-, -(alk)a-S(O)-(alk)b-, -(alk)a-S(O)2-(alk)b-, -(alk)a-OC(O)-(alk)b-, -(alk)a-C(O)O-(alk)b-, -(alk)a-OC(S)-(alk)b-, -(alk)a-C(S)O-(alk)b-, -(alk)a-C(O)NRC-(alk)b-, -(alk)a-C(S)NRC-(alk)b-, -(alk)a-S(O)2NRC-(alk)b-, -(alk)a-NRCC(O)-(alk)b-, -(alk)a-NRCC(S)-(alk)b-, -(alk)a-NRCS(O)2-(alk)b-, -(alk)a-NRCC(O)O-(alk)b-, -(alk)a-NRCC(S)O-(alk)b-, -(alk)a-OC(O)NRC-(alk)b-, -(alk)a-OC(S)NRC-(alk)b-, -(alk)a-NRCC(O)NRD-(alk)b-, -(alk)a-NRCC(S)NRD-(alk)b-, and -(alk)a-NRCS(O)2NRD-(alk)b-;
a and b are independently selected from the group consisting of 0, 1, 2, 3, and 4;
alk is independently selected from the group consisting of C1-5 alkylene, C1-5 alkenylene, and C1-5 alkynylene, each of which is optionally substituted with 1-3 substituents independently selected from the group consisting of H, halogen, —OH, NH2, CF3, C1-5 alkyl, —CH2(NH2), —CN, —NO2, —C(O)OH, —S(O)2NH2, —C(O)NH2, —C(O)CH3, —SH, imidazolyl, pyrazolyl, methylimidazolyl, methylpyrazolyl, —O—C1-5 alkyl, —S—C1-5 alkyl, —NH—C1-5 alkyl, and —N(C1-5 alkyl)2, wherein the C1-5 alkyl groups are independently optionally substituted with 1-3 groups selected from the group consisting of halogen, —OH, —NH2, C1-4 alkyl, CF3, —CH2(NH2), —CN, —NO2, —C(O)OH, —S(O)2NH2, —C(O)NH2, —C(O)CH3, —SH, —SCH3, imidazolyl, pyrazolyl, methylimidazolyl, and methylpyrazolyl.

RC and RD, at each occurrence, are independently selected from the group consisting of hydrogen, C1-3 alkyl, C3-6 cycloalkyl, 5-10 membered heterocycle, aryl, and 5-10 membered heteroaryl, wherein the alkyl, cycloalkyl, heterocycle, aryl, and heteroaryl are each independently optionally substituted with 1-3 substituents selected from the group consisting of halogen, C1-3 alkyl, OH, NH2, NH—C1-3 alkyl, N(C1-3 alkyl)2, CF3, C1-6 alkyl, —CH2(NH2), —CN, —NO2, —C(O)OH, —S(O)2NH2, —C(O)NH2, —C(O)CH3, —SH, —SCH3, imidazolyl, pyrazolyl, methylimidazolyl, and methylpyrazolyl.

X at each occurrence is independently selected from the group consisting of:

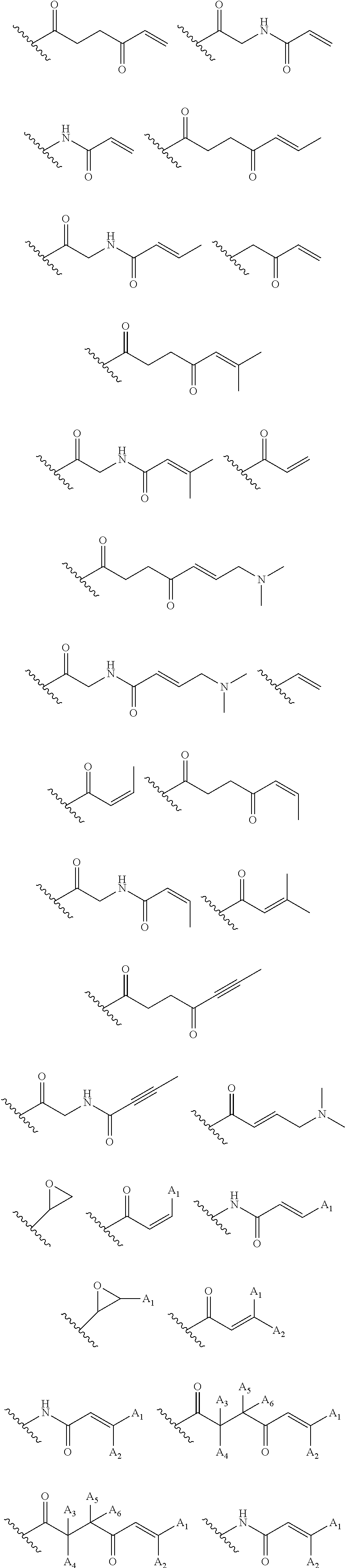

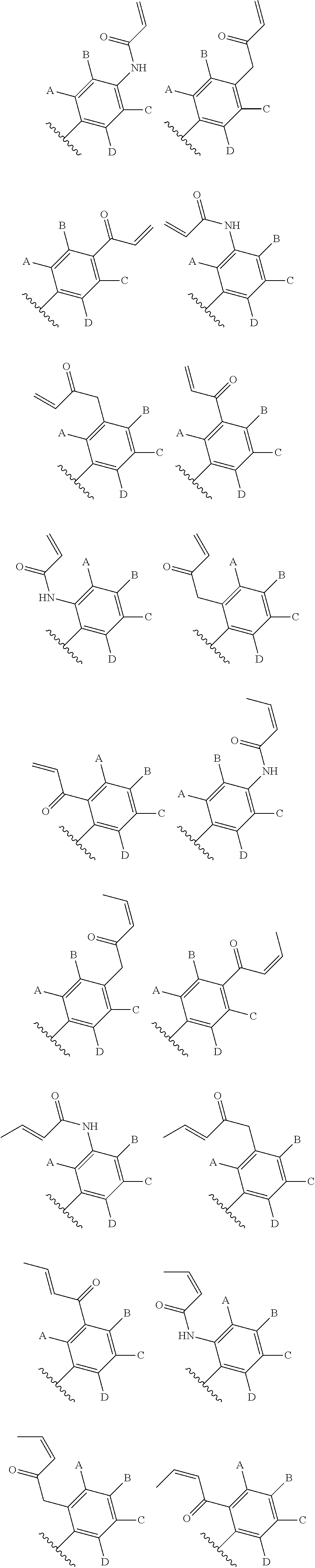

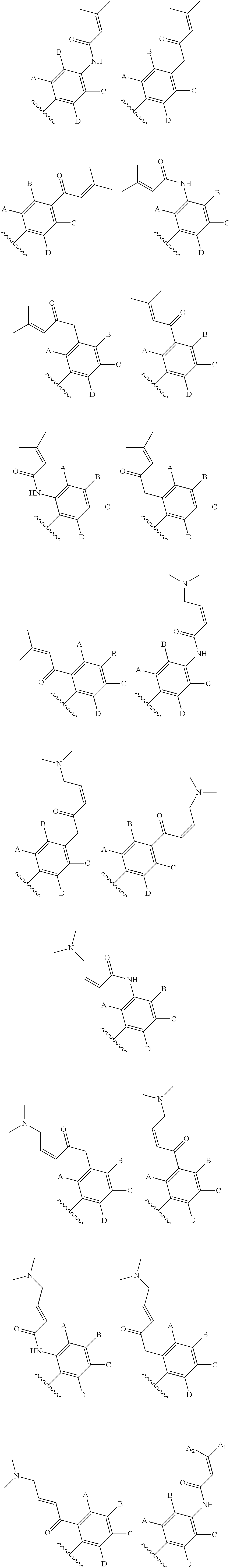

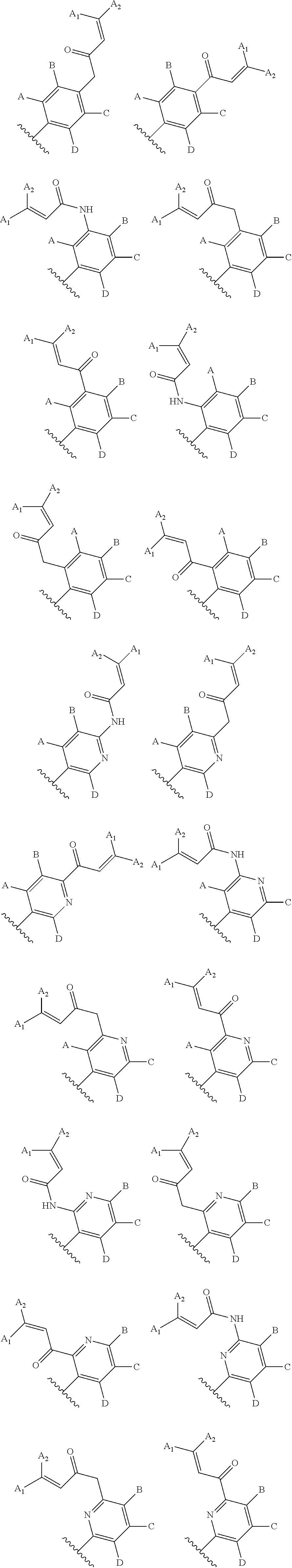

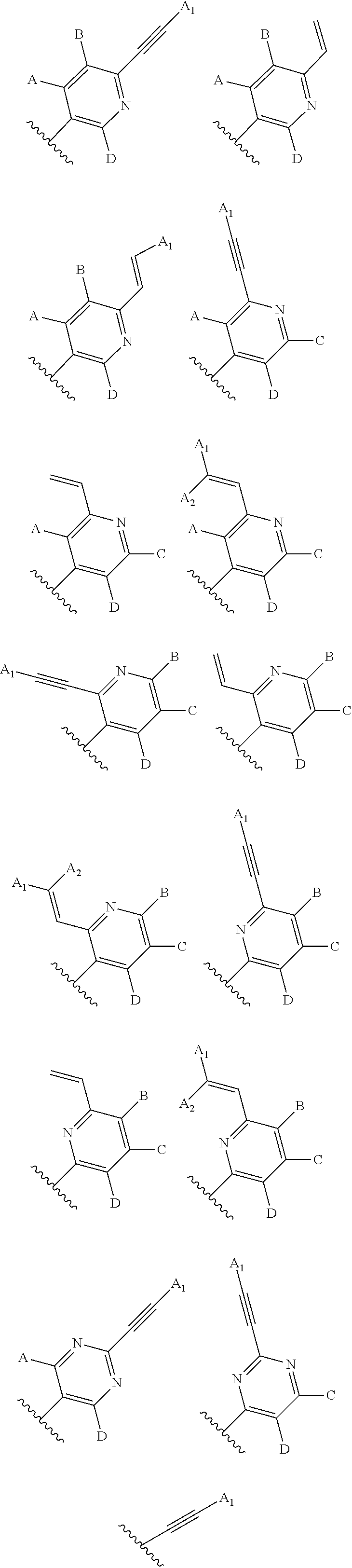

wherein A, B, C, and D at each occurrence are independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for A, B, C, and D are 1-3 substituents indpendently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

A1, A2, A3, A4, A5, and A6 at each occurrence may be independently selected from the group consisting of H, halogen, CF3, —OH, —NH2, —SH, —SCH3, —CN, —NO2, —CH2(NH2), —C(O)OH, —S(O)2NH2, —C(O)NH2, —C(O)CH3, NHC(O)—C1-6 alkyl, N(C1-3 alkyl)C(O)—C1-6 alkyl, OC(O)NH2, OC(O)NH(CH3), OC(O)N(CH3)2, imidazolyl, pyrazolyl, methylimidazolyl, methylpyrazolyl, optionally substituted C1-6 alkyl, optionally substituted C2-6 alkenyl, optionally substituted C2-6 alkynyl, optionally substituted C3-6 cycloalkyl, optionally substituted 5-10 membered heterocycle, optionally substituted aryl, and optionally substituted 5-10 membered heteroaryl,
wherein the optional substituents for A1, A2, A3, A4, A5, and A6 are 1-3 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, —S—CH3, optionally substituted C1-3 alkyl, and optionally substituted C3-6 cycloalkyl,
wherein the C1-3 alkyl and C3-6 cycloalkyl optional substituents are 1-2 substituents independently selected from the group consisting of halogen, OH, NH2, CH3, CF3, —CN, —NO2, —C(O)OH, —S(O)2NH2, —C(O)NH2, —CH2NH2, —C(O)CH3, SH, and —S—CH3.

The disclosure contemplates using all combinations of the various substituents. Thus, any combination of the above-mentioned substituents falling within the structural formula Compound 1-1 to Compound 1-5 can be used.

In some embodiments, the ARCS are selected from the group consisting of narrow generic structures Compound 1-6 to Compound 1-11,

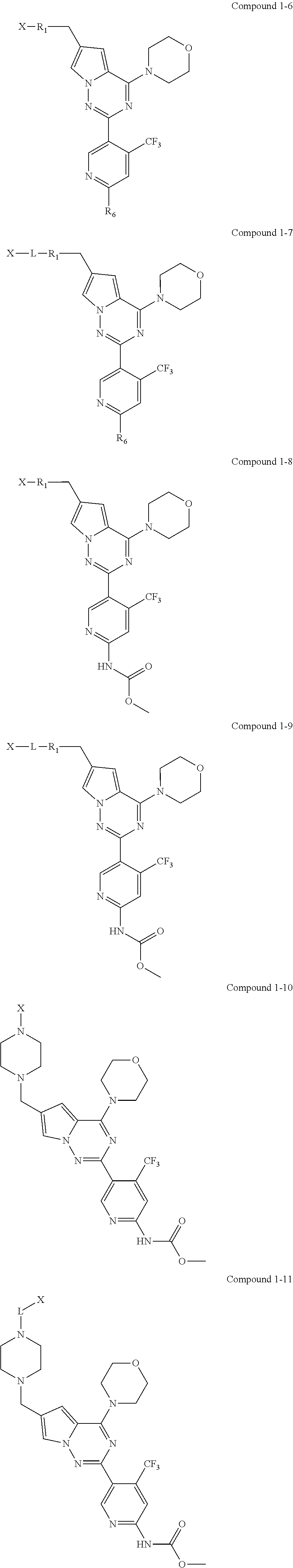

or a pharmaceutically acceptable salt thereof.

In some embodiments, the ARCS may have a structure of

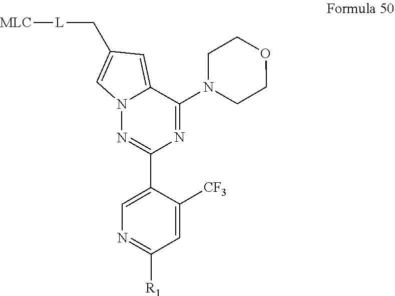

or a pharmaceutically acceptable salt thereof, wherein L is a linker selected from the group consisting of

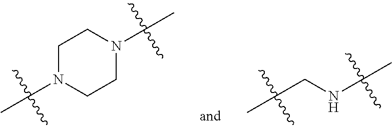

wherein either end can be connected to the CLM; R1 is selected from the group consisting of

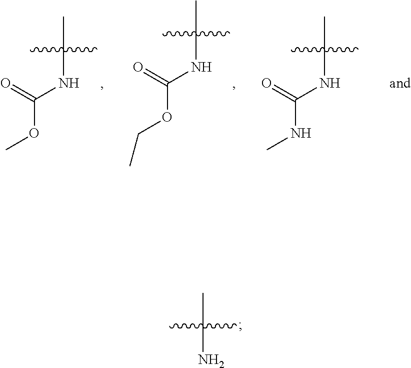

and CLM is selected from the group consisting of

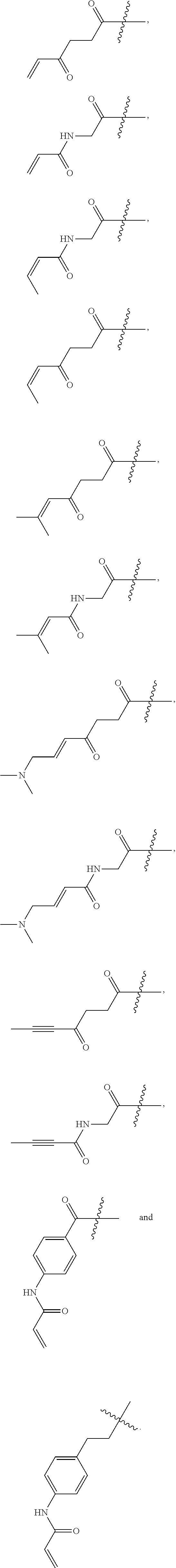

In some embodiments, the ARCS may have a structure of

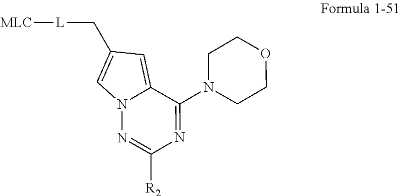

or a pharmaceutically acceptable salt thereof, wherein L, the linker is

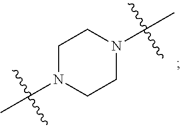

R2 is selected from the group consisting of

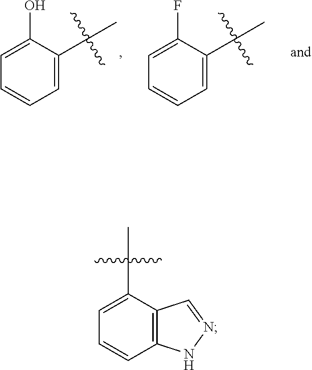 [SMILES: *c1ccccc1O ; *c1ccccc1F ; *c1cccc2[nH]ncc12]

and CLM is selected from the group consisting of

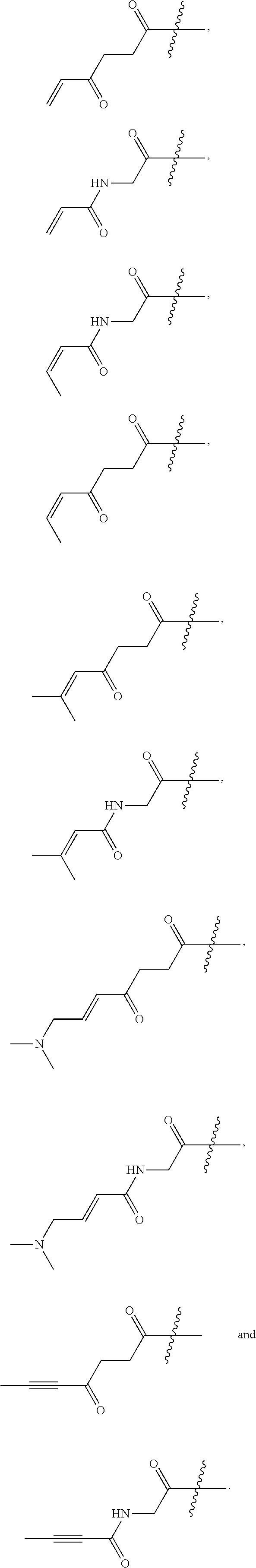

### Additional Therapeutically Active Agents

In one embodiment, the present invention provides a combination therapy comprising a compound of formula having the formula:

(FCB)a-(L)b-(CLM)c,
or a pharmaceutically acceptable salt thereof, and/or their subgenera, or Compound I, or salts, solvates, esters and/or prodrugs thereof disclosed herein can be used in a combination with at least one additional therapeutically active agents or therapy (combination therapy).

The following therapeutics, therapeutically active agents, and therapy may be employed in conjunction with the administration of the compounds described above.

In a specific embodiment, the additional therapeutically active agent is a RAS inhibitor. In another embodiment, the additional therapeutically active agent is a KRAS inhibitor. In a specific embodiment, the additional therapeutically active agent is a class of compound or agent as described or summarized in Kwan, A. K., Piazza, G. A., Keeton, A. B. et al. The path to the clinic: a comprehensive review on direct KRASG12C inhibitors. J Exp Clin Cancer Res 41, 27 (2022), which is incorporated by reference herein in its entirety. In a specific embodiment, the additional therapeutically active agent is an inhibitor of a KRAS mutant. In a specific embodiment, the KRAS mutants is G12C. In a specific embodiment, the additional therapeutically active agent covalently binds to KRAS-G12C. In another specific embodiment, the KRAS inhibitor is selected from one or more of the group consisting of sotorasib, adagrasib, ARS-3248, LY3499446, LY3537982, GDC-6036, D-1553, JDQ443, and BI 1823911. In a specific embodiment, the KRAS inhibitor is selected from one or more of the group consisting of sotorasib and adagrasib. In another embodiment, the KRAS inhibitor is adagrasib.

In one embodiment, the at least one additional therapeutically active agent is a MEK inhibitor, such as a MEK1 and/or MEK2 inhibitor. In another embodiment, the MEK inhibitor or inhibitor of the RAS/RAF/MEK/MAPK signaling pathway, is MEK162, XL518, Refametinib, Pimasertib, selumetinib, GSK1120212, PD-325901, E6201, GDC-0623, CH5126766 AZD-6244, CL-1040 and/or TAK-733. In another embodiment, the at least one additional therapeutically active agent is a BRAF inhibitor. In a specific embodiment, the BRAF inhibitor is Sorafenib, Vemurafenib (Zelboraf), dabrafenib (Tafinlar), and/or encorafenib (Braftovi). In another embodiment, the at least one additional therapeutically active agent is an ERK inhibitor. In a specific embodiment, the ERK inhibitor is CC-90003, GDC-0994, DEL-22379, XMD8-92, ADTL-EI1712, MK-8353, BVD-523, LY3214996, KO-947, ONC201, AZD0364, HH2710, and/or LT1462.

In another embodiment, the at least one additional therapeutically active agent is a PI3K/AKT inhibitor or a PI3K/AKT pathway inhibitor. Dysregulation of the PI3K/Akt pathway is frequently observed in human cancers. PI3K activation leads to the Akt activation and subsequent activation of downstream effectors such as mTOR and others may be associated with resistance to both chemotherapeutic agents and target agents. Accordingly, the at least one additional therapeutically active agent is a PI3K/AKT inhibitor or a PI3K/AKT pathway inhibitor, i.e., an inhibitor in the pathway such as inhibiting a downstream effector such as RHEB or mTOR, including mTORC1 and/or MTORC2 inhibitors. In some embodiments, the PI3K/Akt inhibitor is selected from the group consisting of SC66, MK-2206, AZD5363, AZD8835, AZD8186, BKM120, XL147, GDC0941, GSK1059615, PX-866, CAL-101, KRX0401, VQD-002, XL418, PX316, BYL719, BVD-523, PA-79, ZSTK474, BAY80-6946, B591, TG-100-115, RIDR-PI-103, TAK-117, GSK2636771, CAL-101, PWT-143, AMG319, YY-20394, IBI-376, TGR-1202, RP6530, GDC-0032, IPI-145, CDZ173, IPI-549, BEZ-235, GDC-0980, PF-05212384, SF1126, GSK458, LY3023414, PQR309, GDC0084, XL765, ARQ092, BAY1125976, TAS117, GSK2110183, GDC0068, GSK2141795, GSK690693, and/or PF-04691502.

In some embodiments, the pharmaceutical combinations and/or compositions of present disclosure further comprise least one additional therapeutically active agent that is a PI3K/Akt downstream mTOR pathway inhibitor such as zotarolimus, umirolimus, temsirolimus, sirolimus, sirolimus NanoCrystal, everolimus, biolimus A9, ridaforolimus, rapamycin, TCD-10023, DE-109, MS R001, MS R002, MS-R003, (−)-rapamycin, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, and/or ABTL-0812. In some embodiments, the pharmaceutical combinations and/or compositions of present disclosure further comprise an at least one additional therapeutically active agent that is a Rheb inhibitor. In a specific embodiment, the Rheb inhibitor is HY-124798.

In a specific embodiment, the adagrasib may be administered or provided in a pharmaceutical composition in a dosage amount ranging from about 200 mg to about 2000 mg, about 300 mg to about 1900 mg, about 400 mg to about 1800 mg, about 500 mg to about 1700 mg, about 600 mg to about 1600 mg, about 700 mg to about 1500 mg, about 800 mg to about 1400 mg, about 900 mg to about 1300 mg, about 1,000 mg to about 1,200 mg, about 200 mg to about 2000 mg, or about 400 mg to about 1000 mg, or about 500 mg to about 700 mg. In a specific embodiment, the dose is administered once, twice, or three times a day. In a specific embodiment the specific dose range is administered to a subject twice a day.

### Combination Therapy

In one embodiment, the present invention provides a method of treating a condition associated with cell proliferation in a patient in need thereof. In one embodiment, the present invention provides a method of treating cancer or tumors. The method comprises co-administering to a patient in need thereof a therapeutically effective amount of at least one compound of formula:

(FCB)a-(L)b-(CLM)c,
or a pharmaceutically acceptable salt thereof, and/or their subgenera, or Compound I, or a pharmaceutically acceptable salt or solvate thereof and at least one additional therapeutically active agent. In a specific embodiment, the additional therapeutically active agent is a RAS inhibitor. In another embodiment, the additional therapeutically active agent is a KRAS inhibitor. In a specific embodiment, the additional therapeutically active agent is a class of compound or agent as described or summarized in Kwan, A. K., Piazza, G. A., Keeton, A. B. et al. The path to the clinic: a comprehensive review on direct KRASG12C inhibitors. J Exp Clin Cancer Res 41, 27 (2022), which is incorporated by reference herein in its entirety. In a specific embodiment, the additional therapeutically active agent is an inhibitor for a KRAS mutant. In a specific embodiment, the KRAS mutant is G12C. In a specific embodiment, the additional therapeutically active agent covalently binds to KRAS-G12C. In another specific embodiment, the KRAS inhibitor is selected from one or more of the group consisting of sotorasib, adagrasib, ARS-3248, LY3499446, LY3537982, GDC-6036, D-1553, JDQ443, and BI 1823911. In a specific embodiment, the KRAS inhibitor is selected from one or more of the group consisting of sotorasib and adagrasib. In another embodiment, the KRAS inhibitor is adagrasib

In a specific embodiment, the combination is compound I and adagrasib.

In one embodiment, the combination is compound I and at least one additional therapeutically active agent is a MEK inhibitor, such as a MEK1 and/or MEK2 inhibitor. In another embodiment, the MEK inhibitor or inhibitor of the RAS/RAF/MEK/MAPK signaling pathway, is MEK162, XL518, Refametinib, Pimasertib, selumetinib, GSK1120212, PD-325901, E6201, GDC-0623, CH5126766 AZD-6244, CL-1040 and/or TAK-733. In another embodiment, the at least one additional therapeutically active agent is a BRAF inhibitor. In a specific embodiment, the BRAF inhibitor is Sorafenib, Vemurafenib (Zelboraf), dabrafenib (Tafinlar), and/or encorafenib (Braftovi). In another embodiment, the at least one additional therapeutically active agent is a ERK inhibitor. In a specific embodiment, the ERK inhibitor is CC-90003, GDC-0994, DEL-22379, XMD8-92, ADTL-EI1712, MK-8353, BVD-523, LY3214996, KO-947, ONC201, AZD0364, HH2710, and/or LTT462.

In another embodiment, the combination is compound I and at least one additional therapeutically active agent, including a PI3K/AKT inhibitor or a PI3K/AKT pathway inhibitor. In one embodiment, the at least one additional therapeutically active agent is a PI3K/AKT inhibitor or a PI3K/AKT pathway inhibitor, i.e., an inhibitor in the pathway such as inhibiting a downstream effector such as RHEB or mTOR, including mTORC1 and/or MTORC2 inhibitors. In some embodiments, the PI3K/Akt inhibitor is selected from the group consisting of SC66, MK-2206, AZD5363, AZD8835, AZD8186, BKM120, XL147, GDC0941, GSK1059615, PX-866, CAL-101, KRX0401, VQD-002, XL418, PX316, BYL719, BVD-523, PA-79, ZSTK474, BAY80-6946, B591, TG-100-115, RIDR-PI-103, TAK-117, GSK2636771, CAL-101, PWT-143, AMG319, YY-20394, IBI-376, TGR-1202, RP6530, GDC-0032, IPI-145, CDZ173, IPI-549, BEZ-235, GDC-0980, PF-05212384, SF1126, GSK458, LY3023414, PQR309, GDC0084, XL765, ARQ092, BAY1125976, TAS117, GSK2110183, GDC0068, GSK2141795, GSK690693, and/or PF-04691502.

In some embodiments, the pharmaceutical combinations is compound I and the at least one additional therapeutically active agent is a PI3K/Akt downstream mTOR pathway inhibitor such as zotarolimus, umirolimus, temsirolimus, sirolimus, sirolimus NanoCrystal, everolimus, biolimus A9, ridaforolimus, rapamycin, TCD-10023, DE-109, MS R001, MS R002, MS-R003, (−)-rapamycin, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, or ABTL-0812. In some embodiments, the at least one additional therapeutically active agent is a Rheb inhibitor. In a specific embodiment, the Rheb inhibitor is HY-124798.

In a specific embodiment, the combinations described herein may be co-administered. The term “co-administration” or “coadministration” refers to administration of (a) formula (FCB)a-(L)b-(CLM)c, or a pharmaceutically acceptable salt, ester, solvate and/or prodrug thereof, and/or their subgenera, or Compound I, and (b) at least one additional therapeutically active agent, together in a coordinated fashion. For example, the co-administration can be simultaneous administration, sequential administration, overlapping administration, interval administration, continuous administration, or a combination thereof. In one embodiment, a compound of formula (FCB)a-(L)b-(CLM)c, and/or their subgenera, or Compound I, or a pharmaceutically acceptable salt, ester, solvate and/or prodrug thereof, thereof and at least one additional therapeutically active agent are formulated into a single dosage form. In another embodiment, formula a compound of formula (FCB)a-(L)b-(CLM)c, and/or their subgenera, or Compound I, or a pharmaceutically acceptable salt, ester, solvate and/or prodrug thereof and at least one additional therapeutically active agent are provided in a separate dosage forms.

In one embodiment, the co-administration is carried out for one or more treatment cycles. By “treatment cycle”, it is meant a pre-determined period of time for co-administering the compound of a compound of formula (FCB)a-(L)b-(CLM)c, and/or their subgenera, or Compound I, or a pharmaceutically acceptable salt, ester, solvate and/or prodrug thereof and at least one therapeutically active agent, such as a KRAS inhibitor. Typically, the patient is examined at the end of each treatment cycle to evaluate the effect of the present combination therapy. In one embodiment, the co-administration is carried out for 1 to 48 treatment cycles. In another embodiment, the co-administration is carried out for 1 to 36 treatment cycles. In another embodiment, the co-administration is carried out for 1 to 24 treatment cycles.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about a month-long treatment cycle. In another embodiment, the treatment cycle has from about 4 to about 6 weeks.

Depending on the patient's condition and the intended therapeutic effect, the dosing frequency for each of the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, may vary from once per day to six times per day. That is, the dosing frequency may be once per day, twice per day, three times per day, four times per day, five times per day, or six times per day. In some embodiments, dosing frequency may be one to six times per week or one to four times per month. In one embodiment, dosing frequency may be once a week, once every two weeks, once every three weeks, once every four weeks, or once a month.

There may be one or more void days in a treatment cycle. By “void day”, it is meant a day when neither the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered. In other words, none of the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered on a void day. Any treatment cycle must have at least one non-void day. By “non-void day”, it is meant a day when at least one of the compounds of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered.

By “simultaneous administration”, it is meant that the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, are administered on the same day. For the simultaneous administration, the compound and at least one therapeutically active agent can be administered at the same time or one at a time.

In one embodiment of the simultaneous administration, the compound of formula (FCB)a-(L)b-(CLM)c, and/or their subgenera, or Compound I, or a pharmaceutically acceptable salt, ester, solvate and/or prodrug thereof, is administered from 1 to 4 times per day, 1 to 4 times per week, once every two weeks, once every three weeks, once every four weeks or 1 to 4 times per month; and the at least one additional therapeutically active agent is administered 1 to 4 times per day, 1 to 4 times per week, once every two weeks, once every three weeks, once every four weeks or 1 to 4 times per month. In another embodiment of the simultaneous administration, the compound of formula (FCB)a-(L)b-(CLM)c, and/or their subgenera, or Compound I, or a pharmaceutically acceptable salt, ester, solvate and/or prodrug thereof, is administered once a week, once every two weeks, once every three weeks, once every four weeks, or once a month; and the at least one additional therapeutically active agent is administered 1 to 4 times per day, 1 to 4 times per week, once every two weeks, once every three weeks, once every four weeks or 1 to 4 times per month.

By “sequential administration”, it is meant that during a period of two or more days of continuous co-administration without any void day, only one of the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered on any given day.

In one embodiment of the sequential administration, the compound of formula (FCB)a-(L)b-(CLM)c, and/or their subgenera, or Compound I, or a pharmaceutically acceptable salt, ester, solvate and/or prodrug thereof, is administered from 1 to 4 times per day, 1 to 4 times per week, once every two weeks, once every three weeks, once every four weeks or 1 to 4 times per month; and at least one additional therapeutically active agent is administered 1 to 4 times per day, 1 to 4 times per week, once every two weeks, once every three weeks, once every four weeks or 1 to 4 times per month. In another embodiment of the sequential administration of the compound of formula (FCB)a-(L)b-(CLM)c, and/or their subgenera, or Compound I, or a pharmaceutically acceptable salt, ester, solvate and/or prodrug thereof, is administered from once a week, once every two weeks, once every three weeks, once every four weeks, or once a month; and at least one additional therapeutically active agent is administered 1 to 4 times per day, 1 to 4 times per week, once every two weeks, once every three weeks, once every four weeks or 1 to 4 times per month.

By “overlapping administration”, it is meant that during a period of two or more days of continuous co-administration without any void day, there is at least one day of simultaneous administration and at least one day when only one of the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered.

By “interval administration”, it is meant a period of co-administration with at least one void day. By “continuous administration”, it is meant a period of co-administration without any void day. The continuous administration may be simultaneous, sequential, or overlapping, as described above.

In the present method, the co-administration comprises oral administration, parenteral administration, or a combination thereof. Examples of the parenteral administration include but are not limited to intravenous (IV) administration, intraarterial administration, intramuscular administration, subcutaneous administration, intraosseous administration, intrathecal administration, or a combination thereof. The compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, can be independently administered orally or parenterally. In one embodiment, the compound of formula (FCB)a-(L)b-(CLM)c, and/or their subgenera, or Compound I, or a pharmaceutically acceptable salt, ester, solvate and/or prodrug thereof and at least one therapeutically active agent such as a KRAS inhibitor, is administered parenterally. The parenteral administration may be conducted via injection or infusion.

In one embodiment of the present method, Compound I is provided for use in combination therapy with at least one additional therapeutically active agent, such as a KRAS inhibitor. In a specific embodiment, the KRAS inhibitor is adagrasib.

In one embodiment, Compound I and adagrasib are orally, subcutaneously, or intravenously administered.

### Pharmaceutical Formulations

In another embodiment, the present invention provides a pharmaceutical composition and/or combination comprising a therapeutically effective amount of a compound of formula (FCB)a-(L)b-(CLM)c, and/or their subgenera, or Compound I, or a pharmaceutically acceptable salt, ester, solvate and/or prodrug thereof, as disclosed herein, as the active ingredient, combined with a pharmaceutically acceptable excipient or carrier. The excipients are added to the formulation for a variety of purposes.

In some embodiments, the compound of formula (FCB)a-(L)b-(CLM)c, and/or their subgenera, or Compound I, or a pharmaceutically acceptable salt, ester, solvate and/or prodrug thereof and at least one therapeutically active agent may be formulated into a single pharmaceutical composition and/or combination. In a specific embodiment, the single pharmaceutical composition and/or combination is Compound I and a KRAS inhibitor. In a specific embodiment, the KRAS inhibitor is adagrasib.

In some embodiments, the compound of formula (FCB)a-(L)b-(CLM)c, and/or their subgenera, or Compound I, or a pharmaceutically acceptable salt, ester, solvate and/or prodrug thereof and at least one therapeutically active agent are formulated into a separate pharmaceutical composition and/or combination comprising a pharmaceutically acceptable excipient or a carrier.

In a specific embodiment, Compound I, or a pharmaceutically acceptable salt, ester, solvate and/or prodrug thereof and at least one therapeutically active agent may be formulated into a single pharmaceutical composition and/or combination composition. In a specific embodiment, the additional therapeutically active agent is a RAS inhibitor. In another embodiment, the additional therapeutically active agent is a KRAS inhibitor. In a specific embodiment, the additional therapeutically active agent is a class of compound or agent as described or summarized in Kwan, A. K., Piazza, G. A., Keeton, A. B. et al. The path to the clinic: a comprehensive review on direct KRASG12C inhibitors. J Exp Clin Cancer Res 41, 27 (2022), which is incorporated by reference herein in its entirety. In a specific embodiment, the additional therapeutically active agent is an inhibitor for a KRAS mutant. In a specific embodiment, the KRAS mutants is G12C. In a specific embodiment, the additional therapeutically active agent covalently binds to KRAS-G12C. In another specific embodiment, the KRAS inhibitor is selected from one or more of the group consisting of sotorasib, adagrasib, ARS-3248, LY3499446, LY3537982, GDC-6036, D-1553, JDQ443, and BI 1823911. In a specific embodiment, the KRAS inhibitor is selected from one or more of the group consisting of sotorasib and adagrasib. In another embodiment, the KRAS inhibitor is adagrasib.

In a specific embodiment, the adagrasib may be provided in a pharmaceutical composition in a dosage amount ranging from about 100 mg to about 2000 mg, about 300 mg to about 1900 mg, about 400 mg to about 1800 mg, about 500 mg to about 1700 mg, about 600 mg to about 1600 mg, about 700 mg to about 1500 mg, about 800 mg to about 1400 mg, about 900 mg to about 1300 mg, about 1,000 mg to about 1,200 mg, about 200 mg to about 2000 mg, or about 400 mg to about 1000 mg, or about 500 mg to about 700 mg.

In a specific embodiment, Compound I, or a pharmaceutically acceptable salt, ester, solvate and/or prodrug thereof and at least one therapeutically active agent may be formulated into a separate pharmaceutical composition and/or combination composition. In a specific embodiment, the separate pharmaceutical compositions may be included in the form of a kit. In a specific embodiment, the additional therapeutically active agent is a RAS inhibitor. In another embodiment, the additional therapeutically active agent is a KRAS inhibitor. In a specific embodiment, the additional therapeutically active agent is a class of compound or agent as described or summarized in Kwan, A. K., Piazza, G. A., Keeton, A. B. et al. The path to the clinic: a comprehensive review on direct KRASG12C inhibitors. J Exp Clin Cancer Res 41, 27 (2022), which is incorporated by reference herein in its entirety. In a specific embodiment, the additional therapeutically active agent is an inhibitor for a KRAS mutant. In a specific embodiment, the KRAS mutant is G12C. In a specific embodiment, the additional therapeutically active agent covalently binds to KRAS-G12C. In another specific embodiment, the KRAS inhibitor is selected from one or more of the group consisting of sotorasib, adagrasib, ARS-3248, LY3499446, LY3537982, GDC-6036, D-1553, JDQ443, and BI 1823911. In a specific embodiment, the KRAS inhibitor is selected from one or more of the group consisting of sotorasib and adagrasib. In another embodiment, the KRAS inhibitor is adagrasib. In a specific embodiment, the adagrasib may be provided in a pharmaceutical composition in a dosage amount ranging from about 100 mg to about 2000 mg, about 300 mg to about 1900 mg, about 400 mg to about 1800 mg, about 500 mg to about 1700 mg, about 600 mg to about 1600 mg, about 700 mg to about 1500 mg, about 800 mg to about 1400 mg, about 900 mg to about 1300 mg, about 1,000 mg to about 1,200 mg, about 200 mg to about 2000 mg, or about 400 mg to about 1000 mg, or about 500 mg to about 700 mg. In a specific embodiment, the kit or separate compositions may include Compound I and adagrasib.

In one embodiment of the combinations or compositions of the present invention, the at least one additional therapeutically active agent is a MEK inhibitor, such as a MEK1 and/or MEK2 inhibitor. In another embodiment, the MEK inhibitor or inhibitor of the RAS/RAF/MEK/MAPK signaling pathway, is MEK162, XL518, Refametinib, Pimasertib, selumetinib, GSK1120212, PD-325901, E6201, GDC-0623, CH5126766 AZD-6244, CL-1040 and/or TAK-733. In another embodiment, the at least one additional therapeutically active agent is a BRAF inhibitor. In a specific embodiment, the BRAF inhibitor is Sorafenib, Vemurafenib (Zelboraf), dabrafenib (Tafinlar), and/or encorafenib (Braftovi). In another embodiment, the at least one additional therapeutically active agent is a ERK inhibitor. In a specific embodiment, the ERK inhibitor is CC-90003, GDC-0994, DEL-22379, XMD8-92, ADTL-EI1712, MK-8353, BVD-523, LY3214996, KO-947, ONC201, AZD0364, HH2710, and/or LTT462.

In one embodiment of the combinations or compositions, the at least one additional therapeutically active agent is a PI3K/AKT inhibitor or a PI3K/AKT pathway inhibitor. In one embodiment, the at least one additional therapeutically active agent is a PI3K/AKT inhibitor or a PI3K/AKT pathway inhibitor, i.e., an inhibitor in the pathway such as inhibiting a downstream effector such as RHEB or mTOR, including mTORC1 and/or MTORC2 inhibitors. In some embodiments, the PI3K/Akt inhibitor is selected from the group consisting of SC66, MK-2206, AZD5363, AZD8835, AZD8186, BKM120, XL147, GDC0941, GSK1059615, PX-866, CAL-101, KRX0401, VQD-002, XL418, PX316, BYL719, BVD-523, PA-79, ZSTK474, BAY80-6946, B591, TG-100-115, RIDR-PI-103, TAK-117, GSK2636771, CAL-101, PWT-143, AMG319, YY-20394, IBI-376, TGR-1202, RP6530, GDC-0032, IPI-145, CDZ173, IPI-549, BEZ-235, GDC-0980, PF-05212384, SF1126, GSK458, LY3023414, PQR309, GDC0084, XL765, ARQ092, BAY1125976, TAS117, GSK2110183, GDC0068, GSK2141795, GSK690693, and PF-04691502.

In one embodiment of the combinations or compositions, the least one additional therapeutically active agent is a PI3K/Akt downstream mTOR pathway inhibitor such as zotarolimus, umirolimus, temsirolimus, sirolimus, sirolimus NanoCrystal, everolimus, biolimus A9, ridaforolimus, rapamycin, TCD-10023, DE-109, MS R001, MS R002, MS-R003, (−)-rapamycin, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, or ABTL-0812. In some embodiments, the at least one additional therapeutically active agent is a Rheb inhibitor. In a specific embodiment, the Rheb inhibitor is HY-124798.

Pharmaceutical acceptable excipients may be added to the composition/formulation. For example, diluents may be added to the formulations of the present invention. Diluents increase the bulk of a solid pharmaceutical composition and/or combination and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions and/or combinations that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition and/or combination in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition and/or combination, the composition and/or combination is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions and/or combinations may also be dyed using any pharmaceutically acceptable colorant to improve their appearance and/or facilitate patient identification of the product and unit dosage level.

In liquid pharmaceutical compositions and/or combinations may be prepared using the excipients where the components are dissolved or suspended in a liquid carrier such as water, vegetable oil, alcohol, polyethylene glycol, propylene glycol, or glycerin.

Liquid pharmaceutical compositions and/or combinations may contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions and/or combinations may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition and/or combination may also contain a buffer such as guconic acid, lactic acid, citric acid or acetic acid, sodium guconate, sodium lactate, sodium citrate, or sodium acetate. Selection of excipients and the amounts used may be readily determined by the formulation scientist based upon experience and consideration of standard procedures and reference works in the field.

The solid compositions and/or combination of the present invention include powders, granulates, aggregates and compacted compositions and/or combinations. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions, aerosols and elixirs.

The dosage form of the present invention may be a capsule containing the composition and/or combination, preferably a powdered or granulated solid composition and/or combination of the invention, within either a hard or soft shell. The shell may be made from gelatin and optionally contain a plasticizer such as glycerin and sorbitol, and an opacifying agent or colorant.

A composition and/or combination for tableting or capsule filling may be prepared by wet granulation. In wet granulation, some or all of the active ingredients and excipients in powder form are blended and then further mixed in the presence of a liquid, typically water that causes the powders to clump into granules. The granulate is screened and/or milled, dried and then screened and/or milled to the desired particle size. The granulate may be tableted, or other excipients may be added prior to tableting, such as a glidant and/or a lubricant.

A tableting composition and/or combination may be prepared conventionally by dry blending. For example, the blended composition and/or combination of the actives and excipients may be compacted into a slug or a sheet and then comminuted into compacted granules. The compacted granules may subsequently be compressed into a tablet.

As an alternative to dry granulation, a blended composition and/or combination may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting; however, they are not subjected to a final tableting step.

The active ingredient and excipients may be formulated into compositions and/or combinations and dosage forms according to methods known in the art.

### Therapeutic Use

The compounds and the pharmaceutical compositions of the present disclosure find use in any number of methods. For example, in some embodiments the combinations and the pharmaceutical compositions are useful in methods for modulating a phosphoinositide 3-kinase (PI3K) and/or RAS, such as KRAS. In embodiments, modulating phosphoinositide 3-kinase (PI3K) activity and/or KRAS activity is in a mammalian cell. In embodiments, modulating phosphoinositide 3-kinase (PI3K) and/or KRAS can be in a subject in need thereof (e.g., a mammalian subject) and for treatment of a condition or disease described herein, including diseases or conditions wherein irreversible inhibition of PI3K and/or KRAS provides therapeutic benefit to a subject having the disease or condition.

In one embodiment, the modulating PI3K is binding to PI3K. In another embodiment, the modulating KRAS is by binding to KRAS. In other embodiments, the modulating PI3K is inhibiting PI3K, including irreversibly inhibiting the activity of PI3K. In other embodiments, the modulating KRAS is inhibiting KRAS, including irreversibly inhibiting the activity of KRAS. In embodiments, the inhibiting PI3K is inhibiting PI3Ka, including irreversibly inhibiting the activity of PI3Ka, for example by forming a covalent bond with a cysteine residue on PI3Ka.

In embodiments, modulating phosphoinositide 3-kinase (PI3K) activity is for treatment of diseases or conditions wherein irreversible inhibition of PI3K provides therapeutic benefit to a subject having the disease or condition. In embodiments, modulating phosphoinositide 3-kinase (PI3K) activity is for treatment of cancer with a mutation in the PIK3CA gene. In another embodiment, the KRAS is a mutant. In specific embodiment, the KRAS is KRASG12C.

In embodiments of the present disclosure, a method of reducing, inhibiting, or ameliorating cell proliferation in a patient in need thereof is provided. In embodiments, the reducing, inhibiting, or ameliorating in the method disclosed herein, is in vivo. In another embodiment, the reducing, inhibiting, or ameliorating is in vitro. In embodiments, the cells in the method disclosed herein, are a cancer cells. In embodiments of the present disclosure, a method for reducing or preventing tumor growth, comprising contacting tumor cells with a compound or pharmaceutical composition as disclosed herein. In one embodiment, reducing or preventing tumor growth includes reduction in tumor volume. In one embodiment, reducing or preventing tumor growth includes complete elimination of tumors. In one embodiment, reducing or preventing tumor growth includes stopping or halting the existing tumor to grow. In one embodiment, reducing or preventing tumor growth includes reduction in the rate of tumor growth.

The present invention also provides treatment of disorders related to proliferation of cells. In one embodiment, the method comprises contacting cancer and/or tumor cells with the compound of formula (FCB)a-(L)b-(CLM)c, and/or their subgenera, or Compound I, or a pharmaceutically acceptable salt, ester, solvate and/or prodrug thereof and at least one therapeutically active agent, as disclosed herein. In a specific embodiment, the compound is Compound I and the one therapeutically active agent is a KRAS inhibitor, for example an irreversible inhibitor of KRAS. In embodiments, the KRAS inhibitor is an irreversible inhibitor of KRASG12C. In a specific embodiment, the KRAS inhibitor is adagrasib.

In another embodiment, the compound is Compound I and the additional therapeutically active agent is a RAS inhibitor or preferably a KRAS inhibitor. In another embodiment, the additional therapeutically active agent is an inhibitor for a KRAS mutant, e.g., an irreversible inhibitor of a KRAS mutant. In a specific embodiment, the additional therapeutically active agent is specific to the KRAS mutant G12C. In another specific embodiment, the additional therapeutically active agent covalently binds to KRAS-G12C.

In another specific embodiment, the additional therapeutically active agent is a KRAS inhibitor selected from one or more of the group consisting of sotorasib, adagrasib, ARS-3248, LY3499446, LY3537982, GDC-6036, D-1553, JDQ443, and BI 1823911. In another specific embodiment, the KRAS inhibitor is selected from one or more of the group consisting of sotorasib and adagrasib.

In one embodiment of the methods described herein, the at least one additional therapeutically active agent is a MEK inhibitor, such as a MEK1 and/or MEK2 inhibitor. In another embodiment, the MEK inhibitor or inhibitor of the RAS/RAF/MEK/MAPK signaling pathway, is MEK162, XL518, Refametinib, Pimasertib, selumetinib, GSK1120212, PD-325901, E6201, GDC-0623, CH5126766 AZD-6244, CL-1040 and/or TAK-733. In another embodiment, the at least one additional therapeutically active agent is a BRAF inhibitor. In a specific embodiment, the BRAF inhibitor is Sorafenib, Vemurafenib (Zelboraf), dabrafenib (Tafinlar), and/or encorafenib (Braftovi). In another embodiment, the at least one additional therapeutically active agent is a ERK inhibitor. In a specific embodiment, the ERK inhibitor is CC-90003, GDC-0994, DEL-22379, XMD8-92, ADTL-EI1712, MK-8353, BVD-523, LY3214996, KO-947, ONC201, AZD0364, HH2710, and/or LTT462.

In one embodiment of the methods described herein, the at least one additional therapeutically active agent is a PI3K/AKT inhibitor or a PI3K/AKT pathway inhibitor. In another embodiment, the at least one additional therapeutically active agent is a PI3K/AKT inhibitor or a PI3K/AKT pathway inhibitor, i.e., an inhibitor in the pathway such as inhibiting a downstream effector such as RHEB or mTOR, including mTORC1 and/or MTORC2 inhibitors. In some embodiments, the PI3K/Akt inhibitor is selected from the group consisting of SC66, MK-2206, AZD5363, AZD8835, AZD8186, BKM120, XL147, GDC0941, GSK1059615, PX-866, CAL-101, KRX0401, VQD-002, XL418, PX316, BYL719, BVD-523, PA-79, ZSTK474, BAY80-6946, B591, TG-100-115, RIDR-PI-103, TAK-117, GSK2636771, CAL-101, PWT-143, AMG319, YY-20394, IBI-376, TGR-1202, RP6530, GDC-0032, IPI-145, CDZ173, IPI-549, BEZ-235, GDC-0980, PF-05212384, SF1126, GSK458, LY3023414, PQR309, GDC0084, XL765, ARQ092, BAY1125976, TAS117, GSK2110183, GDC0068, GSK2141795, GSK690693, and/or PF-04691502.

In one embodiment of the methods described herein, the least one additional therapeutically active agent is a PI3K/Akt downstream mTOR pathway inhibitor such as zotarolimus, umirolimus, temsirolimus, sirolimus, sirolimus NanoCrystal, everolimus, biolimus A9, ridaforolimus, rapamycin, TCD-10023, DE-109, MS R001, MS R002, MS-R003, (−)-rapamycin, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, and/or ABTL-0812. In some embodiments, the methods include pharmaceutical combinations and/or compositions of the present disclosure wherein at least one additional therapeutically active agent is a Rheb inhibitor. In a specific embodiment, the Rheb inhibitor is HY-124798.

In one embodiment, the compound and one or more active agents of the pharmaceutical combinations are administered together. In a specific embodiment, the compound and one or more active agents are in the same dosage form. In another embodiment, the compound and one or more active agents are in different dosage forms but administered at the same time. In another embodiment, the compound and one or more active agents are in different dosage forms and one is administered to a subject before the other dosage form.

The present invention also comprises the administration of the pharmaceutical combinations described herein to a subject. In other specific embodiments, the methods may include treating or inhibiting cancer in a subject. In a another specific embodiment, the methods may include treating or ameliorating a cell proliferation disorder in a subject, the method comprising administering to a subject in need thereof a therapeutically effective amount of a compound that forms a covalent bond with a kinase or pseudokinase or a pharmaceutically acceptable salt thereof, and administering at least one additional therapeutically active agent before, during, or after the subject has been administered a compound that forms a covalent bond with a kinase or pseudokinase. In a specific embodiment, the kinase is PI3-kinase (PI3K).

In another embodiment of treating or ameliorating a cell proliferation disorder in a subject, the cell proliferation disorder is cancer. In a specific embodiment, the cancer is selected from the group consisting of: heme cancer, colorectal cancer, ovarian cancer, breast cancer, cervical cancer, lung cancer, liver cancer, colon cancer, pancreatic cancer, cancer of the lymph nodes, colon cancer, small intestine, prostate cancer, brain cancer, cholangiocarcinoma, gallbladder carcinoma, cancer of the head and neck, bone cancer, Ewing's sarcoma, skin cancer, kidney cancer, and cancer of the heart. In another embodiment, the cancer is selected from the group consisting of wherein the cancer is selected from the group consisting of breast cancer, ovarian cancer, pancreatic cancer, colorectal cancer, colon cancer, small intestine and lung cancer. In another embodiment of the present invention, the subject is human.

In one embodiment, the present invention provides a method of reducing or inhibiting cell proliferation, and/or a method of treating cancer comprising co-administering to a patient in need thereof a therapeutically effective amount of at least one compound of formula (FCB)a-(L)b-(CLM)c, and/or their subgenera, or Compound I, or a pharmaceutically acceptable salt, ester, solvate and/or prodrug thereof and at least one therapeutically active agent. The present invention also provides methods of treating, preventing, ameliorating and/or alleviating the progression of disorders or conditions characterized by cell proliferation in a subject. More particularly, the methods of the present invention involve administration of an effective amount of the quinolone compounds described herein, in a subject to treat a disorder or a condition characterized by cell proliferation.

As used herein, administering can be effected or performed using any of the various methods known to those skilled in the art. The compound of formula (FCB)a-(L)b-(CLM)c, and/or their subgenera, or Compound I, or a pharmaceutically acceptable salt, ester, solvate and/or prodrug thereof and at least one therapeutically active agent, can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles.

Further, the compound of formula (FCB)a-(L)b-(CLM)c, and/or their subgenera, or Compound I, or a pharmaceutically acceptable salt, ester, solvate and/or prodrug thereof and at least one therapeutically active agent, can be administered to a localized area in need of treatment. This can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The form in which the compound of formula (FCB)a-(L)b-(CLM)c, and/or their subgenera, or Compound I, or a pharmaceutically acceptable salt, ester, solvate and/or prodrug thereof and at least one therapeutically active agent, is administered (e.g., syrup, elixir, capsule, tablet, foams, emulsion, gel, etc.) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compound of formula (FCB)a-(L)b-(CLM)c, and/or their subgenera, or Compound I, or a pharmaceutically acceptable salt, ester, solvate and/or prodrug thereof and at least one therapeutically active agent, disclosed herein can be administered together with other biologically active agents, such as anticancer agents, analgesics, anti-inflammatory agents, anesthetics and other agents which can control one or more symptoms or causes of a disorder or a condition characterized by cell proliferation.

In one embodiment, the compound of formula (FCB)a-(L)b-(CLM)c, and/or their subgenera, or Compound I, or a pharmaceutically acceptable salt, ester, solvate and/or prodrug thereof and at least one therapeutically active agent, can be administered together with a second therapeutically active agent or more.

Additionally, administration can comprise administering to the subject a plurality of dosages over a suitable period of time. Such administration regimens can be determined according to routine methods, upon a review of the instant disclosure.

In settings of a gradually progressive disorder or condition characterized by cell proliferation, the compound of formula (FCB)a-(L)b-(CLM)c, and/or their subgenera, or Compound I, or a pharmaceutically acceptable salt, ester, solvate and/or prodrug thereof and at least one therapeutically active agent, are generally administered on an ongoing basis. In certain settings administration of a compound of formula (FCB)a-(L)b-(CLM)c, and/or their subgenera, or Compound I, or a pharmaceutically acceptable salt, ester, solvate and/or prodrug thereof and at least one therapeutically active agent, disclosed herein can commence prior to the development of disease symptoms as part of a strategy to delay or prevent the disease. In other settings the compound of formula (FCB)a-(L)b-(CLM)c, and/or their subgenera, or Compound I, or a pharmaceutically acceptable salt, ester, solvate and/or prodrug thereof and at least one therapeutically active agent, disclosed herein is administered after the onset of disease symptoms as part of a strategy to slow or reverse the disease process and/or part of a strategy to improve cellular function and reduce symptoms.

It will be appreciated by one of skill in the art that dosage range will depend on the particular compound of formula (FCB)a-(L)b-(CLM)c, and/or their subgenera, or Compound I, or a pharmaceutically acceptable salt, ester, solvate and/or prodrug thereof and at least one therapeutically active agent, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when Compound I disclosed herein are used in accordance with the present application.

An effective amount the compound of formula (FCB)a-(L)b-(CLM)c, and/or their subgenera, or Compound I, or a pharmaceutically acceptable salt, ester, solvate and/or prodrug thereof and at least one therapeutically active agent, disclosed herein comprise amounts sufficient to produce a measurable biological response. Actual dosage levels of active ingredients of the present application can be varied so as to administer an amount of the compound of formula (FCB)a-(L)b-(CLM)c, and/or their subgenera, or Compound I, or a pharmaceutically acceptable salt, ester, solvate and/or prodrug thereof and at least one therapeutically active agent that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure. The following examples further illustrate the present invention but should not be construed as in any way limiting its scope.

### EXAMPLES

The disclosure now being generally described, it will be more readily understood by reference to the following examples which are included merely for purposes of illustration of certain aspects and embodiments of the present invention and are not intended to limit the invention.

The examples below are intended to be exemplary and efforts have been made to ensure accuracy with respect to numbers used (for example, amounts, temperature, etc.), but some experimental errors and deviations should be accounted for within the knowledge of a person skilled in the art. Unless indicated otherwise, temperature is in degrees Centigrade. Reagents were purchased from commercial suppliers such as Sigma-Aldrich, Alfa Aesar, or TCI, and were used without further purification unless otherwise indicated.

### Example 1: Synthesis and Characterization of methyl (5-(6-((4-(acryloylglycyl)piperazin-1-yl)methyl)-4-morpholinopyrrolo[2,1-f][1,2,4]triazin-2-yl)-4-(trifluoromethyl)pyridin-2-yl)carbamate (Compound I)

### Synthesis of Ethyl 1-amino-5-carbamoyl-1H-pyrrole-3-carboxylate (1)

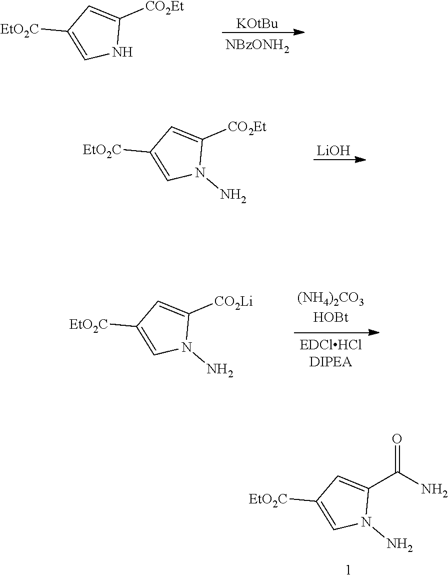

Diethyl 1-amino-1H-pyrrole-2,4-dicarboxylate. A 20 L reaction vessel was loaded with diethyl 1H-pyrrole-2,4-dicarboxylate (1002 g, 1 equiv, 4.507 mol) and NMP (7.8 L). Then potassium tert-butoxide (557.3 g, 1.1 equiv, 4.967 mol) was added, the mixture became pink, and the temperature reached 37° C. The mixture was stirred until all potassium tert-butoxide had dissolved and it was allowed to cool down to 21° C. Subsequently, O-(4-nitrobenzoyl) hydroxylamine (839.6 g, 1.02 equiv, 4.610 mol) was added portion-wise (exothermic, reached 36° C.). Reaction mixture turns into a dark purple suspension. The reaction mixture was stirred at 45° C. overnight (T slowly drops, mixture turns orange). A sample was taken after 18 h and diluted with ACN/water for HPLC analysis. Then, a solution of sodium dithionite (478.6 g, 0.6 equiv, 2.749 mol) in water (2.5 L) was slowly added keeping the temperature below 30°. The reaction mixture was transferred into a 50 L separation funnel. Toluene (15 L) and water (5 L) were added, and the phases were separated. The aqueous phase was extracted with toluene (3×1 L). The combined org phases were washed with water (5×1 L), sat. sodium bicarbonate (5×1 L) and brine (1 L), dried over sodium sulfate, filtered, and concentrated to give diethyl 1-amino-1H-pyrrole-2,4-dicarboxylate (1077 g, 3.90 mol, 86%) as an orange oil (81% QNMR purity), which solidified upon standing. ¹H NMR (299 MHz, DMSO-d6) δ 7.52 (d, J=2.1 Hz, 1H), 7.03 (d, J=2.1 Hz, 1H), 6.51 (d, J=1.8 Hz, 2H), 4.34-4.06 (m, 4H), 1.38-1.14 (m, 6H). LCMS (ESI): found 227.0 [M+H]⁺ (calculated 227.1 [M+H]⁺).

Lithium 1-amino-4-(ethoxycarbonyl)-1H-pyrrole-2-carboxylate. A 10 L reaction vessel was charged with diethyl 1-amino-1H-pyrrole-2,4-dicarboxylate (1077 g, 1 equiv, 3.90 mol), ethanol (3.8 L) and water (1.9 L). Lithium hydroxide monohydrate (163.9 g, 56 wt %, 0.99 equiv, 3.831 mol) was added and the mixture was stirred at 60° C. The conversion was monitored by LCMS analysis. The reaction was stopped after 6 hours and cooled down to RT. The mixture was diluted with 2 L toluene and layers were separated. The aqueous phase was washed with toluene (3×750 ml) and concentrated at the rotary evaporator at 60° C. The resulting solids was suspended in TBME (2 L), filtered and washed with TBME (1 L). Lithium 1-amino-4-(ethoxycarbonyl)-1H-pyrrole-2-carboxylate (853.1 g, 3.50 mol, 90%) was obtained as a pale-yellow solid (83% QNMR purity). ¹H NMR (400 MHz, DMSO-D6) δ 7.37 (s, 2H), 7.09 (d, J=2.2 Hz, 1H), 6.63 (d, J=2.2 Hz, 1H), 4.12 (q, J=7.1 Hz, 2H), 1.22 (t, J=7.1 Hz, 3H). LCMS (ESI): found 199.0 [M−Li+2H]⁺ (calculated 199.1 [M−Li+2H]⁺).

Ethyl 1-amino-5-carbamoyl-1H-pyrrole-3-carboxylate (1). To a suspension of lithium 1-amino-4-(ethoxycarbonyl)-1H-pyrrole-2-carboxylate (835.8 g, 1 equiv, 3.399 mmol) in a mixture of DMF (3.4 L)/MeTHF (8.5 mL) was added ammonium carbonate (2939 g, 9.00 equiv, 30.59 mol) followed by HOBt (1041 g, 2.0 equiv, 6.797 mol), EDCI HCl (1303 g, 2.0 equiv, 6.797 mol) and DIPEA (2.96 L, 5 equiv, 16.99 mol). Adding the reagents causes an endotherm. The flask was stirred at room temperature (suspension) for 3 days. The conversion was monitored by LCMS analysis. The solids were filtered off and washed with MeTHF (2 L) and the filtrate was concentrated at 60° C. The crude material (2 kg) was redissolved in MeTHF (10 L) and washed with a saturated solution of sodium bicarbonate (2×2 L). Three phases were formed. The top phase (product fraction) was separated, dried over sodium sulfate, filtered and concentrated to dryness. The resulting crude material (908 g) was recrystallized from EtOH (1.8 L). The solids were filtered, washed with EtOH (200 mL) and dried to give the first crop of ethyl 1-amino-5-carbamoyl-1H-pyrrole-3-carboxylate (67 g) as a white solid. The mother liquor was concentrated (794 g) and purified by column chromatography (2 kg silica, gradient: DCM to 5% MeOH). The purified material (505 g) was recrystallized from EtOH (750 mL) and washed with EtOH (100 mL) to give a second crop of ethyl 1-amino-5-carbamoyl-1H-pyrrole-3-carboxylate (68 g). The concentrated mother liquor (435 g) was purified by column chromatography once more (6 kg silica, gradient: DCM to 5% MeOH). Recrystallizing the product fractions resulted into an additional crop 61 g of ethyl 1-amino-5-carbamoyl-1H-pyrrole-3-carboxylate. All crops merged gave ethyl 1-amino-5-carbamoyl-1H-pyrrole-3-carboxylate (1) (195.7 g, 992.4 mmol, 29%) as a white solid. ¹H NMR (299 MHz, DMSO-d6) δ 7.96 (s, 1H), 7.36 (d, J=2.0 Hz, 1H), 7.31 (s, 1H), 7.13 (d, J=2.1 Hz, 1H), 6.86 (d, J=1.8 Hz, 2H), 4.18 (q, J=7.1 Hz, 2H), 1.25 (t, J=7.1 Hz, 3H). LCMS (ESI): found 198.0 [M+H]⁺ (calculated 198.1 [M+H]⁺).

### Synthesis of 6-Amino-4-(trifluoromethyl) nicotinaldehyde

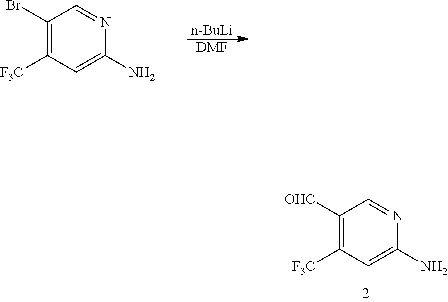

6-Amino-4-(trifluoromethyl) nicotinaldehyde (2). A 5 L three-necked flask, equipped with a thermometer and under nitrogen, was charged with 5-bromo-4-(trifluoromethyl)-2-pyridylamine (200.0 g, 1 equiv, 829.8 mmol) and dry THE (2000 mL). The solution was cooled down to −70° C. A 2.5 M solution of n-butyllithium in hexane (995.8 mL, 3.00 equiv, 2.49 mol) was added dropwise over 90 min, keeping the temperature below 60° C. The mixture was stirred at −70° C. for 15 min. Then, DMF (160.6 mL, 2.50 equiv, 2.074 mol) was added dropwise over 45 min, keeping the temperature below −60° C. The mixture was stirred at −70° C. for 30 min. The mixture was warmed-up to −40° C. and carefully quenched with water (74.8 mL, 5 equiv, 4.149 mol). The resulting solution was left to warm up to room temperature and stirred overnight. The mixture was further diluted with water/ethyl acetate (1 L/1 L) and transferred to a separating funnel. The organic layer was washed with water (5×300 mL). The combined aqueous phases were extracted with ethyl acetate (3×300 mL). The combined organic phases were washed with brine (300 mL), dried over sodium sulfate, filtered and concentrated until a small volume of solvent is left and a solid is formed. The mixture was cooled down to rt and diluted with heptane (300 mL). The mixture was filtered, the solid washed with heptane and dried to give 6-amino-4-(trifluoromethyl) nicotinaldehyde (2) (51.5 g, 271 mmol, 33%) as an orange solid. ¹H NMR (299 MHz, DMSO-d6) δ 9.80 (q, J=1.7 Hz, 1H), 8.63 (d, J=2.1 Hz, 1H), 7.73 (s, 2H), 6.84 (d, J=2.0 Hz, 1H). LCMS (ESI): found 191.0 [M+H]⁺ (calculated 191.0 [M+H]⁺).

### Step 1: Synthesis of Ethyl 2-(6-amino-4-(trifluoromethyl) pyridin-3-yl)-4-oxo-3,4-dihydropyrrolo[2,1-f][1,2,4]triazine-6-carboxylate (3)

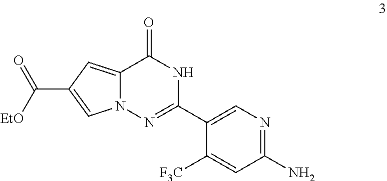 [SMILES: CCOC(=O)c1cc2c(=O)[nH]c(-c3cnc(N)cc3C(F)(F)F)nn2c1]

A 3 L 3-neck flask was equipped with a condenser, temperature probe and a mechanical stirrer and charged with a solution of ethyl 1-amino-5-carbamoyl-1H-pyrrole-3-carboxylate (1) (195.7 g, 1.00 equiv, 962.7 mmol) and 6-amino-4-(trifluoromethyl) nicotinaldehyde (2) (192.7 g, 1.00 equiv, 962.7 mmol) in DMSO (1.9 L). Then cupric chloride dihydrate (213.4 g, 1.30 equiv, 633.4 mmol) was added and the mixture was stirred at 100° C. for 18 hours. The mixture was cooled down to room temperature and poured onto ice water (10 L) causing the precipitation of the product. The suspension was stirred for 30 min before filtering (Buchner filter). The pale brown filter cake was washed with water (3×1 L) and TBME (3×1 L). The solids were stripped with toluene (3×1 L) on a rotary evaporator to remove residues of water. Ethyl 2-(6-amino-4-(trifluoromethyl) pyridin-3-yl)-4-oxo-3,4-dihydropyrrolo[2,1-f][1,2,4] triazine-6-carboxylate (3) (340.6 g, 760.0 mmol, 79%) was obtained as a solid. ¹H NMR (299 MHz, DMSO-d6) δ 12.20 (s, 1H), 8.08 (d, J=1.8 Hz, 1H), 7.23 (d, J=1.8 Hz, 1H), 7.05 (s, 1H), 6.87 (s, 1H), 4.26 (q, J=6.9 Hz, 2H), 1.29 (t, J=7.1, 3H). LCMS (ESI): found 368.0 [M+H]⁺ (calculated 368.1 [M+H]⁺); 366.0 [M−H]⁻ (calculated 366.1 [M−H]⁻).

### Step 2: Synthesis of Ethyl 2-(6-amino-4-(trifluoromethyl) pyridin-3-yl)-4-morpholinopyrrolo[2,1-f][1,2,4]triazine-6-carboxylate (4)

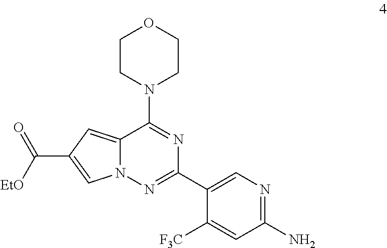 [SMILES: CCOC(=O)c1cc2c(N3CCOCC3)nc(-c3cnc(N)cc3C(F)(F)F)nn2c1]

A 5 L three-necked flask, under nitrogen and equipped with a thermometer, was charged with ethyl 2-(6-amino-4-(trifluoromethyl) pyridin-3-yl)-4-oxo-3,4-dihydropyrrolo[2,1-f][1,2,4]triazine-6-carboxylate (3) (335.6 g, 1 equiv, 749.2 mmol), DMF (2.5 L) and morpholine (400 mL, 6.2 equiv, 2.580 mol). Then PyBOP (606.9 g, 1.55 equiv, 1.166 mol) was added in several portions keeping the temperature around 15° C. (exothermic). The mixture was stirred at room temperature for 48 hours. The reaction mixture was transferred into a 20 L vessel and water (12.5 L) was slowly added. The suspension was filtered over a Buchner filter, washed with water (3×5 L) and TBME (2×5 L). The remaining clay like substance was re-dissolved in ethyl acetate at reflux. The hot suspension was filtered over a glass filter with Celite to remove the copper and the filtrate was concentrated. The remaining solid was suspended in ethyl acetate (1 L) at 60° C. The suspension was centrifuged at 3000 rpm. The resulting solution was decanted and concentrated. The residue was treated with warm ethyl acetate three more times. All organic phases were concentrated, and the obtained solids were filtered, washed with TBME, and dried under vacuum at 60° C. The solids were stripped with toluene (2×1 L) to obtain Ethyl 2-(6-amino-4-(trifluoromethyl) pyridin-3-yl)-4-morpholinopyrrolo[2,1-f][1,2,4]triazine-6-carboxylate (4) (206.5 g, 473.2 mmol, 63%) as a pale-yellow solid with a QNMR purity of 91%. ¹H NMR (300 MHz, cdcl₃) δ 8.56 (s, 1H), 8.09 (d, J=1.6 Hz, 1H), 7.19 (d, J=1.6 Hz, 1H), 6.81 (s, 1H), 4.88 (s, 2H), 4.37 (q, J=7.1 Hz, 2H), 4.08 (t, J=4.8 Hz, 4H), 3.84 (t, J=4.8 Hz, 4H), 1.39 (t, J=7.1 Hz, 3H). LCMS (ESI): found 437.2 [M+H]⁺ (calculated 437.2 [M+H]⁺).

### Step 3: Synthesis of tert-Butyl 4-(2-(6-amino-4-(trifluoromethyl) pyridin-3-yl)-4-morpholinopyrrolo [2,1-f][1,2,4]triazine-6-carbonyl) piperazine-1-carboxylate (5)

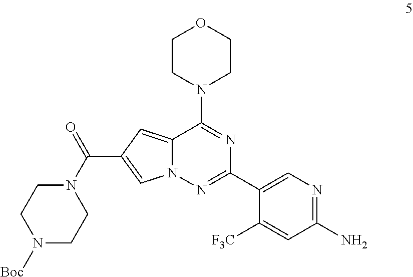 [SMILES: CC(C)(C)OC(=O)N1CCN(C(=O)c2cc3c(N4CCOCC4)nc(-c4cnc(N)cc4C(F)(F)F)nn3c2)CC1]

A 3 L three-necked flask under nitrogen was charged with ethyl 2-(6-amino-4-(trifluoromethyl) pyridin-3-yl)-4-morpholinopyrrolo[2,1-f][1,2,4]triazine-6-carboxylate (4) (206.5 g, 91 wt %, 1 equiv, 473.2 mmol) and THF (2 L). Then tert-butyl piperazine-1-carboxylate (440.7 g, 5.0 equiv, 2.366 mol) and TBD (65.87 g, 1.0 equiv, 473.2 mmol) were added and the solution was stirred at 65° C. for 2 days. The conversion was monitored by LCMS and proton NMR analysis. The reaction mixture was concentrated at 50° C. to remove the majority of THF. The material was dissolved in ethyl acetate (1 L) and washed with a 1M solution of potassium bisulfate (2×500 mL), water (3×500 ml, diluted with brine to enhance separation) and brine (250 mL). The organic phase was dried over sodium sulfate, filtered and concentrated at 50° C. (foaming brown oil). The brown foam was stripped with toluene (1.5 L) to remove traces of ethyl acetate and tert-butyl 4-(2-(6-amino-4-(trifluoromethyl) pyridin-3-yl)-4-morpholinopyrrolo[2,1-f][1,2,4]triazine-6-carbonyl) piperazine-1-carboxylate (5) (382.0 g, 464 mmol, 98%) was obtained as a brown foam with a QNMR purity of 70%. ¹H NMR (300 MHz, cdcl₃) δ 8.52 (s, 1H), 7.77 (d, J=1.6 Hz, 1H), 6.98 (d, J=1.7 Hz, 1H), 6.80 (s, 1H), 4.06 (t, J=4.9 Hz, 4H), 3.82 (t, J=4.9 Hz, 4H), 3.77-3.66 (m, overlaps with a THF signal, but subtraction of another THF signal gives 4H), 3.48 (t, J=5.0 Hz, 4H), 1.47 (m, 27H). LCMS (ESI): found 577.2 [M+H]⁺ (calculated 577.3 [M+H]⁺).

### Step 4: tert-Butyl 4-((2-(6-amino-4-(trifluoromethyl) pyridin-3-yl)-4-morpholinopyrrolo [2,1-f][1,2,4]triazin-6-yl) methyl) piperazine-1-carboxylate (6)

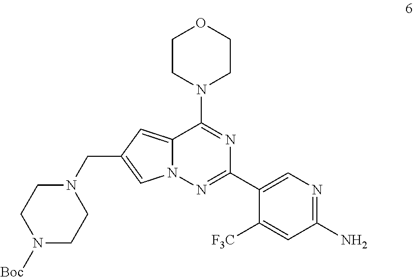 [SMILES: CC(C)(C)OC(=O)N1CCN(Cc2cc3c(N4CCOCC4)nc(-c4cnc(N)cc4C(F)(F)F)nn3c2)CC1]

A 5 L three-necked flask was charged with tert-butyl 4-(2-(6-amino-4-(trifluoromethyl) pyridin-3-yl)-4-morpholinopyrrolo[2,1-f][1,2,4]triazine-6-carbonyl) piperazine-1-carboxylate (5) (271.0 g, 1 equiv, 470.0 mmol) and dry THE (2 L) under nitrogen. The resulting solution was cooled to 0° C. and a solution of TMS-Cl (107.0n mL, 1.80 equiv, 846.0 mmol) in dry THE (100 mL) was added (mixture forms a brown suspension). The mixture was cooled further to −20° C. and a 2.4 M LiAlH₄ in THE (294.0 mL, 1.50 equiv, 705.0 mmol) was added over 80 min. The reaction mixture was stirred at −20° C. for 60 minutes. A 2M solution of Rochelle salt (1 L) was slowly added (very exothermic in the beginning, gas evolution) between −20° C. and −10° C. (the mixture become very thick, a solid is formed after 100 ml addition. The solid slowly dissolves at −8° C. and becomes easier to stir again). The mixture was allowed to warm-up to RT overnight. The organic layer was collected, the aq. layer was extracted with ethyl acetate. The combined organic layers were washed with brine, dried over sodium sulfate, filtered and concentrated. The residue was purified by column chromatography (6 kg silica; eluent: DCM/3.5 M NH₃ in MeOH) to give tert-butyl 4-((2-(6-amino-4-(trifluoromethyl) pyridin-3-yl)-4-morpholinopyrrolo[2,1-f] [1,2,4]triazin-6-yl) methyl) piperazine-1-carboxylate (6) (172.2 g, 306.1 mmol, 65%) as an off-white fluffy solid. ¹H NMR (299 MHz, cdcl₃) δ 8.53 (s, 1H), 7.58 (d, J=1.5 Hz, 1H), 6.80 (s, 1H), 6.67 (d, J=1.6 Hz, 1H), 4.82 (s, 2H), 4.14-3.95 (m, 4H), 3.82 (t, J=4.8 Hz, 4H), 3.58 (s, 2H), 3.44 (t, J=5.3 Hz, 4H), 2.43 (t, J=5.1 Hz, 4H), 1.45 (d, J=1.4 Hz, 9H). LCMS (ESI): found 563.3 [M+H]⁺ (calculated 563.3 [M+H]⁺); 561.2 [M−H]⁻ (calculated 561.3 [M−H]⁻).

### Step 5: tert-Butyl 4-((2-(6-((methoxycarbonyl)amino)-4-(trifluoromethyl) pyridin-3-yl)-4-morpholinopyrrolo[2,1-f][1,2,4]triazin-6-yl) methyl) piperazine-1-carboxylate (7)

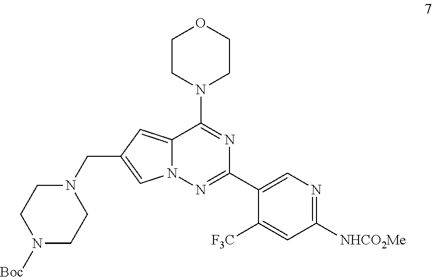

A 3 L three-necked flask was charged with tert-butyl 4-((2-(6-amino-4-(trifluoromethyl)pyridin-3-yl)-4-morpholinopyrrolo[2,1-f][1,2,4]triazin-6-yl)methyl)piperazine-1-carboxylate (6) (172.2 g, 1 Eq, 306.1 mmol), DCM (1.4 L) and pyridine (74 mL, 3.0 Eq, 918.2 mmol) and the solution was cooled down to 0° C. Methyl carbonochloridate (26.1 mL, 1.10 Eq, 336.7 mmol) was added to the reaction mixture over 30 min. The mixture was allowed to slowly warm-up to room temperature and stirred overnight. HPLC analysis showed about 90% conversion. Additional methyl carbonochloridate (2.4 mL, 0.10 Eq, 30.6 mmol) was added dropwise over 5 min and the mixture stirred at room temperature. After 4 h, HPLC analysis showed full conversion reach full conversion. The reaction mixture was poured into cold water (500 ml) and transferred to a separating funnel. The organic layer was collected, washed with water (3×500 ml) and brine, dried over sodium sulfate, filtered and concentrated. The residue was stripped with toluene to give tert-butyl 4-((2-(6-((methoxycarbonyl)amino)-4-(trifluoromethyl) pyridin-3-yl)-4-morpholinopyrrolo[2,1-f][1,2,4]triazin-6-yl) methyl) piperazine-1-carboxylate (7) (174.1 g, 280.5 mmol, 92% yield) as a solid. ¹H NMR (299 MHz, cdcl₃) δ 8.87 (s, 1H), 8.74 (s, 1H), 8.43 (s, 1H), 7.59 (d, J=1.5 Hz, 1H), 6.69 (d, J=1.6 Hz, 1H), 4.05 (t, J=4.8 Hz, 4H), 3.84 (d, J=7.1 Hz, 7H), 3.59 (s, 2H), 3.45 (t, J=5.1 Hz, 4H), 2.44 (t, J=4.9 Hz, 4H), 1.45 (s, 9H). LCMS (ESI): found 621.3 [M+H]⁺ (calculated 621.8 [M+H]⁺); 619.2 [M−H]⁻ (calculated 619.3 [M−H]⁻).

### Step 6: Methyl (5-(4-morpholino-6-(piperazin-1-ylmethyl) pyrrolo[2,1-f][1,2,4] triazin-2-yl)-4-(trifluoromethyl) pyridin-2-yl) carbamate hydrochloride (8)

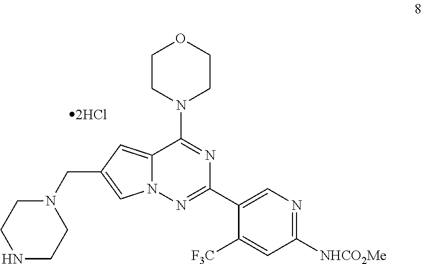

A flask was charged, under nitrogen, with tert-butyl 4-((2-(6-((methoxycarbonyl)amino)-4-(trifluoromethyl)pyridin-3-yl)-4-morpholinopyrrolo[2,1-f][1,2,4]triazin-6-yl)methyl)piperazine-1-carboxylate (7) (172.1 g, 1 Eq, 277.3 mmol) and CPME (1.0 L) (white suspension) and the mixture was cooled down to 6° C. A solution of HCl in CPME (1.5 L, 3.0 molar, 16.2 Eq, 4.50 mol) was added over 30 min (only slightly exothermic) and the mixture stirred at room temperature (yellow precipitate immediately formed) overnight. HPLC analysis showed full conversion. The mixture was filtered, the solid washed with 250 ml CPME and 500 ml TBME. The solid was transferred to a flask, but because it was very sticky (probably hygroscopic) MeOH was used to transfer the solid. The mixture was concentrated at the rotary evaporator and the solid dried to give 198 g of crude product. The material was stripped with toluene (2×1 L) before recrystallizing. The crude material (191 g) was recrystallized from MeOH (300 mL) The solid was collected by filtration, washed with cold MeOH (50 mL) and dried to give methyl (5-(4-morpholino-6-(piperazin-1-ylmethyl) pyrrolo[2,1-f][1,2,4]triazin-2-yl)-4-(trifluoromethyl) pyridin-2-yl) carbamate dihydrochloride (8) (88.8 g, 150 mmol, 54% yield) as a solid. ¹H NMR (400 MHz, DMSO-D6) δ 12.13 (s, 1H), 10.92 (s, 1H), 9.59 (s, 2H), 8.73 (s, 1H), 8.31 (s, 1H), 8.07 (s, 1H), 7.38 (s, 1H), 4.41 (s, 2H), 4.02 (t, J=4.9 Hz, 4H), 3.80-3.69 (m, 8H), 3.69-3.33 (m, 13H), 3.16 (s, 3H). LCMS (ESI): found 521.2 [M+H]⁺ (calculated 521.2 [M+H]⁺).

### Step 7: Methyl (5-(6-((4-(acryloylglycyl) piperazin-1-yl) methyl)-4-morpholinopyrrolo[2,1-f] [1,2,4]triazin-2-yl)-4-(trifluoromethyl) pyridin-2-yl) carbamate (Compound 1)

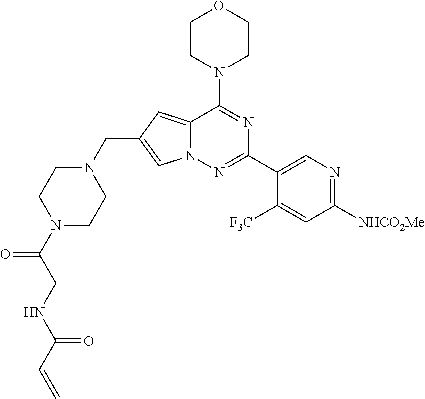

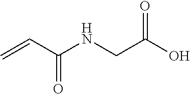

Acryloyl glycine. A 2 L RBF was charged with glycine (200 g, 1.0 Eq, 2.66 mol), deionized water (88 mL) and sodium hydroxide (710 mL, 30% Wt, 2.0 Eq, 5.32 mol). The mixture was cooled to −5° C. before acryloyl chloride (225 mL, 96% Wt, 1.0 Eq, 2.66 mol) was added while maintaining the temperature around 0° C. The mixture was stirred at 0° C. for 1 h. After 1 h the mixture was acidified with HCl (310 mL, 12 molar, 1.4 Eq, 3.72 mol) until pH 2. The mixture was saturated with sodium sulphate (±100 g) and diluted with warm MeTHF (300 mL). Phases were separated, and the aqueous phase was extracted with MeTHF (3×250 mL). The combined organic layers were washed with brine (150 mL), dried over sodium sulphate and concentrated. At a volume of roughly 500 mL, a solvent swap to ethyl acetate (added 500 mL) was performed. After removing 500 mL ethyl acetate, the solvent switch was repeated twice more. The resulting slurry was agitated at room temperature for 1 h before the solids were collected and rinsed with ethyl acetate (2×60 mL), dried under vacuum, and dried further on a rotary evaporator to give Acryloyl glycine (76.00 g, 588.6 mmol, 22% yield) as white solid. ¹H NMR (299 MHz, DMSO-d6) δ 12.58 (s, 1H), 8.42 (t, J=6.0 Hz, 1H), 6.30 (ddd, J 17.1, 10.1, 0.8 Hz, 1H), 6.10 (ddd, J 17.2, 2.3, 0.8 Hz, 1H), 5.62 (ddd, J=10.1, 2.3, 0.8 Hz, 1H), 3.84 (dd, J=6.0, 0.8 Hz, 2H). LCMS (ESI): found 130.1 [M+H]⁺ (calculated 130.1 [M+H]⁺).

To a suspension of methyl (5-(4-morpholino-6-(piperazin-1-ylmethyl) pyrrolo[2,1-f] [1,2,4]triazin-2-yl)-4-(trifluoromethyl) pyridin-2-yl) carbamate dihydrochloride (8) (73.2 g, 1 Eq, 123 mmol) in THE (750 mL) was added DIPEA (100 mL, 4.7 Eq, 416 mmol) and the mixture slowly became a solution and then turbid again. Acryloyl glycine (23.9 g, 1.5 Eq, 185 mmol) was added followed by HATU (93.8 g, 2 Eq, 247 mmol) and the mixture (yellow) was stirred at room temperature. After 20 min the mixture became a black suspension. After 1 h the reaction was finished and diluted with 600 mL of ethyl acetate and quenched with 600 mL of a sat. solution of sodium bicarbonate. The organic phase was separated and washed with a sat. solution of sodium bicarbonate (600 mL) and brine (2×400 mL). The organic phase was dried over sodium sulphate, filtered, and concentrated to give the 147.2 g of crude material as a red sticky oil. The crude material was dissolved in NMP (600 mL) at room temperature and water (1.5 L) was added dropwise. The mixture was stirred for 1 h at room temperature and then filtered off. The residue was extensively washed with water and TBME to remove most of the NMP. The obtained solids were dissolved in DMSO (470 mL) and precipitated by adding water (900 mL). Once again, the residue was extensively washed with water and TBME to remove most of the NMP and DMSO. After drying the material in a circulation oven at 40° C. overnight, methyl (5-(6-((4-(acryloylglycyl) piperazin-1-yl) methyl)-4-morpholinopyrrolo[2,1-f][1,2,4]triazin-2-yl)-4-(trifluoromethyl) pyridin-2-yl) carbamate (Compound) (57.7 g, 91.4 mmol, 74% yield) was obtained as a solid. ¹H NMR (400 MHz, DMSO-D6) δ 10.89 (s, 1H), 8.72 (s, 1H), 8.30 (s, 1H), 8.20 (t, J=5.5 Hz, 1H), 7.75 (d, J=1.5 Hz, 1H), 6.97 (d, J=1.6 Hz, 1H), 6.37 (dd, J 17.1, 10.2 Hz, 1H), 6.09 (dd, J 17.1, 2.2 Hz, 1H), 5.59 (dd, J=10.2, 2.2 Hz, 1H), 4.02 (d, J=5.5 Hz, 2H), 3.98 (t, J=4.9 Hz, 4H), 3.74 (d, J=4.4 Hz, 7H), 3.57 (s, 2H), 3.52-3.37 (m, 4H), 2.39 (dt, J=17.8, 4.9 Hz, 4H). LCMS (ESI): found 632.4 [M+H]⁺ (calculated 632.3 [M+H]⁺); 630.2 [M−H]⁻ (calculated 630.2 [M−H]⁻).

### Example 2: Transfection Protocol and Readout for NanoBRET Screening

Human embryonic kidney 293-H (HEK 293, Gibco 293-H, #11631017) cell lines were maintained in Dulbecco's Modified Eagle Medium, high glucose, pyruvate (DMEM, Gibco, #11995065) supplemented with 10% fetal bovine serum (FBS, Gibco, #10082147) and 1× penicillin-streptomycin (100× solution, Gibco, #15140148) at 37° C. and 5% CO2 in a water-saturated incubator. Cell were trypsinized using 0.05% or 0.25% Trypsin-EDTA solution (Trypsin-EDTA, phenol red, Gibco, #25200056 (0.25%) or #25300054). Opti-MEM media supplemented with 10% fetal bovine serum (Opti-MEM I reduced serum media, no phenol red, Gibco, #11058021) was used for culturing cells overnight for NanoBRET readout experiments.

HEK293 cells were cultivated appropriately prior to assay. The medium from cell flask was removed via aspiration, washed 1× with PBS followed by aspiration, trypsinized, and cells were allowed to dissociate from the flask. Trypsin was neutralized using growth medium and cells were pelleted via centrifugation at 200×g for 5 minutes. The medium was aspirated and the cells were resuspended into a single cell suspension using Opti-MEM I supplemented with 10% FBS. The cell density was adjusted to 2×10⁵/mL in Opti-MEM I supplemented with 10% FBS in a sterile, conical tube. The cells were transfected and aliquoted directly in a 96-well plate for the NanoBRET assay the next day, and therefore, the cells were cultured overnight in Opti-MEM. The cells were also transfected in bulk and dispensed into a 96-well plate to allow cells to adhere to the plate overnight, thereby enabling washout studies.

The lipid:DNA complexes were prepared as follows:

A 10 μg/mL solution of DNA was prepared in Opti-MEM without serum. This solution contains the following ratios of carrier DNA and DNA encoding NanoLuc fused to the biological target. Serial dilution steps may be warranted to accurately dilute the NanoLuc fusion DNA. Added, in order, the following reagents were added to a sterile polystyrene test tube: 1 mL of Opti-MEM without phenol red; 9.0 μg/mL of carrier DNA; 1.0 μg/mL of NanoLuc fusion DNA (for some targets, the amount is less). The reagents were mixed thoroughly. 30 μL of FuGENE HD is added into each mL of DNA mixture to form lipid:DNA complex. Care is taken such that FuGENE HD does not touch the plastic side of the tube and pipetted directly into the liquid in the tube. It is mixed by pipetting up and down 5-10 times and incubated at room temperature for 20 minutes to allow complexes to form. 1 part (e.g. 1 mL) of lipid:DNA complex was mixed with 20 parts (e.g. 20 mL) of HEK293 cells in suspension at 2×10⁵/mL and mixed gently by pipetting up and down 5 times in a sterile, conical tube. Larger or smaller bulk transfections are scaled accordingly, using this ratio. 100 μL cells+lipid:DNA complex was dispensed into a sterile, tissue-culture treated 96-well plate (20,000 cells/well), and incubated at least 16 hours to allow expression. The cells were then incubated in a 37° C.+5% CO2 incubator for >16 hrs. The serially diluted Compound I was prepared at 100× final concentration in 100% DMSO. The serially diluted Compound I stock was prepared in PCR plates. 1 μL per well of 100× serially diluted inhibitor/compound was added to the cells in 96-well plates that have been transiently transfected overnight and mixed by tapping the plate by hand. The plate was incubated at 37° C.+5% CO2 incubator overnight. A 1× solution of substrate mix (500× stock) and appropriate concentration of tracer was prepared in Opti-Mem. The cells were washed by setting a plate washer to the 96 well plate 5× in PBS pH 7.4 by adding 200 μL PBS each time. The cells were incubated at 37° C. for 2 hours. 100 μL of the 1× Substrate-Tracer solution was added and the 96 well plate is gently tapped to mix. The plate on plate reader is read every hour for the next 6 hours. The binding assay results show that Compound I provides greater than 80% inhibition of the PI3K target.

### Example 3: Cell Proliferation Assay

The objective of this study was investigate the effect of Compound I on the cell proliferation of 13 cell lines after 3 days treatment, and determine the IC50 of Compound I in each cell line. The cell lines tested included as follows: UM-UC-3 (bladder), KYSE-410 (HN/Esophagus), SW1463 (rectum), Calu-1 (Lung), NCI-H358 (Lung), SW837 (Rectum), SW756 (Cervix), NCI-H2122 (Lung), NCI-H1373 (Lung), NCI-H1792 (Lung), NCI-H23 (Lung), MIA PaCa-2 (Pancreas), and HC44.

Cells were recovered and maintained in appropriate culture media. The cells were harvested respectively during the logarithmic growth period. The cells were then resuspended and counted using a Vi cell counter (The cell viability be measured by trypan blue exclusion assay). The cells were then diluted and 90 μL cell suspensions were used in 96-well plates according to plate map with final cell density. Two duplicate plates were set up. One is for day 0 reading (T0) and the other was cultured in incubator for reading at the end point. The incubated plates were incubated overnight in humidified incubator at 37° C. with 5% CO2.

At day 0, 10 μL culture medium to each well for T0 reading. 10 μL of culture medium was added to each well for T0 reading. Then, 50 μl CellTiter-Glo® Reagent was added to each well. The contents were then mixed for 2 minutes on an orbital shaker to facilitate cell lysis. The plates were then allowed to incubate at room temperature for 10 minutes to stabilize luminescent signal. Luminescence was then recorded using an EnVision Multi Label Reader. The test compound Compound I, and a control cisplatin, where then diluted at various concentrations from a 10 mM stock solution. Compound I was diluted and 10 μL of each 10× compound I from working solutions for a concentration ranging from 10 μM to about 0.005 μM. Cisplatin was diluted to about 3.33 mM to about 150 nM. The screening plates were then placed back into the incubator for the appropriate treatment time (3 days).

For the endpoint CTG reading, 50 μL of CellTiter-Glo® Reagent were added to each well. The contents were then mixed for 2 minutes on an orbital shaker to facilitate cell lysis. The plate was allowed to incubate at room temperature for 10 minutes to stabilize luminescent signal. Luminescence was then recorded using an EnVision Multi Label Reader. IC50's were determined for each cell line. The assay indicates Compound I has inhibitive effect across multiple cancer cell lines, including a substantial inhibitory effect against NCI-H358 (see Table A).

TABLE A
IC50 Proliferation Assay

| | Absolute IC50 (μM) | Absolute IC50 (μM) |
|---|---|---|
| Cell Line Name | Compound I | Cisplatin |
| UM-UC-3 (bladder) | >10 | 1.8642 |
| KYSE-410 (HN/Esophagus) | 2.0231 | 15.8034 |
| SW1463 (rectum) | 3.8582 | 12.2245 |
| Calu-1 (Lung) | >10 | 10.1785 |
| NCI-H358 (Lung) | 1.69 | 12.4619 |
| SW837 (Rectum) | >10 | 42.4853 |
| SW756 (Cervix) | 7.2855 | 10.2600 |
| NCI-H2122 (Lung) | 3.7651 | 17.787 |
| NCI-H1373 (Lung) | >10 | 8.7241 |
| NCI-H1792 (Lung) | >10 | 4.8783 |
| NCI-H23 (Lung) | >10 | 3.5848 |
| MIA PaCa-2 (Pancreas) | >10 | 7.0279 |
| HCC44 (Lung) | 7.92 | 7.9621 |

### Example 4: Cell Proliferation Assays and Synergy Analysis

The objective of this study was to investigate the effect of Compound I in combination with KRAS inhibitor Adagrasib on the cell proliferation of 13 cell lines after 3 days treatment. The cell lines tested included: UM-UC-3 (bladder), KYSE-410 (HN/Esophagus), SW1463 (rectum), Calu-1 (Lung), NCI-H358 (Lung), SW837 (Rectum), SW756 (Cervix), NCI-H2122 (Lung), NCI-H1373 (Lung), NCI-H1792 (Lung), NCI-H23 (Lung), MIA PaCa-2 (Pancreas), and HCC44 (Lung).

Cells were recovered and maintained in appropriate culture media. The cells were harvested respectively during the logarithmic growth period. The cells were then resuspended and counted using a Vi cell counter (The cell viability was measured by trypan blue exclusion assay). The cells were then diluted and 80 μL of the cell suspensions were added to 96-well plates according to plate map with final cell density. Two duplicate plates were set up. One is for day 0 reading (T0) and the other was cultured in the incubator for reading at the end point. The incubated plates were incubated overnight in humidified incubator at 37° C. with 5% CO2.

At day 0, 20 μL of culture medium was added to each well for T0 reading. 10 μL of culture medium was added to each well for day 0 (T0) reading. Then, 50 μl of CellTiter-Glo® Reagent was added to each well. The contents were then mixed for 2 minutes on an orbital shaker to facilitate cell lysis. The plates were then allowed to incubate at room temperature for 10 minutes to stabilize luminescent signal. Luminescence was then recorded using an EnVision Multi Label Reader. The test compounds (Compound I and Adagrasib) were then diluted at various concentrations from a 10 mM stock solution. Compound I and Adagrasib were diluted and 10 μL of each 10× test compound (Compound I or Adagrasib) working solutions with concentrations ranging from 10 μM to about 0.005 μM were added to the plate according to plate inoculation map. The screening plates were then placed back into the incubator for the appropriate treatment time (3 days).

For the endpoint CTG reading, 50 μL of CellTiter-Glo® Reagent was added to each well. The contents were then mixed for 2 minutes on an orbital shaker to facilitate cell lysis. The plate was allowed to incubate at room temperature for 10 minutes to stabilize luminescent signal. Luminescence was then recorded using an EnVision Multi Label Reader. The surviving rate (%)=(LumTest article−LumMedium control)/(LumNone treated−LumMedium control)×100%.

Synergy scores were calculated by both Bliss independence model (Bliss C. I. (1939) The toxicity of poisons applied jointly, Ann. Appl. Biol., 26, 585-615) and Loewe additivity models (Loewe S. (1953) the problem of synergism and antagonism of combined drugs, ArzneimiettelForschung, 3, 286-290). A score higher than 5 indicates synergy, and a score less than −5 indicates antagonism.

The anti-proliferative activity of Compound I in single and combination treatments on the 13 cell lines are shown in Tables 1A-13A, below. Synergy scores for the two-drug combination treatments in the 13 cell lines calculated by the Bliss Independence model and Loewe additivity model are shown in Tables 1B-13B and Tables 1C-13C, respectfully.

### UM-UC-3 (Bladder Cancer) Cell Lines

TABLE 1A
Average Inhibition (%) (N = 3) of Compound I and Adagrasib as
single agents and combination treatments on UM-UC-3 (bladder) cell lines.

| Ave | | | | | | | | | | |
|---|---|---|---|---|---|---|---|---|---|---|
| Inhibition | Conc. Of Compound I (uM) | | | | | | | | | |
| rate (%) | | 10.0000 | 3.3333 | 1.1111 | 0.3704 | 0.1235 | 0.0412 | 0.0137 | 0.0046 | 0.00 |
| Conc. Of | 2.3928 | 85.20% | 80.12% | 74.17% | 70.26% | 68.48% | 69.86% | 65.93% | 66.96% | 65.01% |
| Adagrasib | 1.1964 | 77.18% | 67.48% | 58.53% | 52.88% | 51.43% | 50.55% | 50.16% | 48.22% | 48.03% |
| (uM) | 0.5982 | 75.87% | 66.32% | 54.39% | 45.56% | 41.48% | 41.03% | 38.98% | 37.68% | 38.83% |
| | 0.2991 | 73.10% | 61.83% | 50.36% | 40.53% | 37.20% | 35.00% | 34.98% | 34.35% | 35.16% |
| | 0.1496 | 73.56% | 60.68% | 47.64% | 39.33% | 33.87% | 33.01% | 30.94% | 29.84% | 33.00% |
| | 0.0000 | 44.82% | 27.43% | 9.37% | 0.18% | −4.04% | −7.94% | −5.07% | −5.05% | −3.85% |

TABLE 1B
Synergy Scores using the Bliss independence model for Compound I and
Adagrasib combination treatments on UM-UC-3 (bladder) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.15 | uM | 0 | 0.22 | 1.33 | 5.32 | 3.57 | 6.21 | 8.35 | 9.3 | 10.53 |
| 0.299 | uM | 0 | 2.46 | 3.1 | 4.99 | 4.67 | 5.25 | 9.12 | 8.89 | 8.88 |
| 0.598 | uM | 0 | 1.94 | 3.25 | 7.06 | 5.12 | 6.62 | 9.83 | 10.72 | 9.62 |
| 1.2 | uM | 0 | 2.82 | 4.77 | 6.65 | 5.51 | 4.76 | 5.63 | 5.2 | 5.85 |
| 2.39 | uM | 0 | 3.71 | 2.69 | 7.63 | 4.88 | 5.18 | 5.88 | 5.51 | 4.5 |

TABLE 1C
Synergy Scores using the Loewe additivity model for Compound I and
Adagrasib combination treatments on UM-UC-3 (bladder) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.15 | uM | 0 | −3.31 | −2.22 | −0.16 | 0.67 | 6.04 | 13.99 | 24.7 | 27 |
| 0.299 | uM | 0 | −0.54 | 0.08 | 0.08 | 2.23 | 5.35 | 14.48 | 22.78 | 25.02 |
| 0.598 | uM | 0 | −1.3 | −0.02 | 2 | 2.33 | 6.07 | 13.84 | 22.38 | 25.14 |
| 1.2 | uM | 0 | 0.21 | 2.14 | 2.49 | 3.26 | 4.38 | 9.11 | 15.83 | 22.09 |
| 2.39 | uM | 0 | 1.89 | 0.74 | 4.32 | 1.89 | 0.63 | −3.7 | −15.93 | −35.42 |

### KYSE-410 (HN/Esophagus Cancer) Cell Lines

TABLE 2A
Average Inhibition (%) (N = 3) of Compound I and Adagrasib as single
agent and combination treatments on KYSE-410 (HN/Esophagus) cell lines.

| Ave | | | | | | | | | | |
|---|---|---|---|---|---|---|---|---|---|---|
| inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 3.8922 | 99.93% | 99.64% | 98.68% | 96.32% | 92.93% | 87.75% | 83.01% | 79.77% | 74.88% |
| Adagrasib | 1.9461 | 97.51% | 95.57% | 90.16% | 78.25% | 72.78% | 62.66% | 54.25% | 49.52% | 42.33% |
| (uM) | 0.9731 | 92.62% | 89.72% | 82.98% | 72.40% | 58.69% | 44.83% | 31.86% | 27.53% | 20.30% |
| | 0.4865 | 88.56% | 84.93% | 76.49% | 62.44% | 46.72% | 29.85% | 18.52% | 11.92% | 8.57% |
| | 0.2433 | 86.72% | 82.64% | 73.20% | 59.64% | 43.70% | 25.90% | 16.04% | 10.05% | 6.87% |
| | 0.0000 | 71.29% | 65.82% | 50.45% | 29.36% | 17.09% | 9.47% | 3.14% | −1.36% | −0.49% |

TABLE 2B
Synergy Scores using the Bliss independence model for Compound I and Adagrasib
combination treatments on KYSE-410 (HN/Esophagus) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.243 | uM | 0 | 4.44 | 6.24 | 10.21 | 20.91 | 25.43 | 19.34 | 14.47 | 13.46 |
| 0.487 | uM | 0 | 4.59 | 7.08 | 12.62 | 22.52 | 27.03 | 21.8 | 16.18 | 14.81 |
| 0.973 | uM | 0 | 8.3 | 9.05 | 16.98 | 24.76 | 28.7 | 22.47 | 16.96 | 15.5 |
| 1.95 | uM | 0 | 7.97 | 10.11 | 14.87 | 20.6 | 18.99 | 18.74 | 15.28 | 14.07 |
| 3.89 | uM | 0 | 5.23 | 7.34 | 10.49 | 13.75 | 14.06 | 11.13 | 8.22 | 7.14 |

TABLE 2C
Synergy Scores using the Loewe additivity model for Compound I and Adagrasib
combination treatments on KYSE-410 (HN/Esophagus) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.243 | uM | 0 | 3.4 | 8.4 | 14.65 | 23.66 | 25.1 | 21.83 | 17.75 | 13.81 |
| 0.487 | uM | 0 | 1.6 | 6.89 | 14.51 | 23.05 | 25.13 | 23.45 | 19.21 | 15.29 |
| 0.973 | uM | 0 | 7.25 | 10.39 | 20.19 | 27.11 | 29.35 | 26.55 | 22.34 | 18.63 |
| 1.95 | uM | 0 | 6.71 | 10.91 | 17.85 | 24.46 | 23.41 | 26.87 | 24.84 | 22.03 |
| 3.89 | uM | 0 | 4.91 | 8.13 | 12.79 | 17.78 | 20.67 | 22.1 | 21.87 | 21.19 |

### SW1463 (Rectal Cancer) Cell Lines

TABLE 3A
Average Inhibition (%) (N = 3) of Compound I and Adagrasib as
single agent and combination treatments on SW1463 (rectum) cell lines.

| Ave | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 0.7662 | 73.57% | 72.35% | 69.09% | 63.28% | 58.76% | 55.06% | 53.19% | 46.80% | 43.06% |
| Adagrasib | 0.3831 | 65.76% | 63.09% | 58.96% | 53.67% | 50.84% | 48.21% | 43.99% | 40.56% | 37.70% |
| (uM) | 0.1916 | 69.06% | 67.52% | 64.43% | 54.70% | 51.40% | 44.11% | 40.20% | 34.44% | 28.39% |
| | 0.0958 | 68.18% | 63.46% | 57.40% | 48.47% | 44.06% | 40.59% | 34.74% | 33.72% | 30.46% |
| | 0.0479 | 66.98% | 63.27% | 54.74% | 46.72% | 41.79% | 36.65% | 27.98% | 25.47% | 20.97% |
| | 0.0000 | 48.42% | 40.35% | 29.53% | 21.76% | 15.74% | 6.01% | 2.64% | 4.57% | 5.05% |

TABLE 3B
Synergy Scores using the Bliss independence model for Compound I and
Adagrasib combination treatments on SW1463 (rectum) cell lines.

| | Compound I | | | | | | | | |
|---|---|---|---|---|---|---|---|---|---|
| | | 0.0046 | 0.0137 | 0.0412 | 0.1235 | 0.3704 | 1.1111 | 3.3333 | 10.0000 |
| Adagrasib | 0 | uM | uM | uM | uM | uM | uM | uM | uM |
| 0 | 0 | 0 | 0 | 0 | 0 | 0 | 0 | 0 | 0 |
| 0.0479 uM | 0 | 0.89 | 4.93 | 10.93 | 8.39 | 8.56 | 10.43 | 10.41 | 7.75 |
| 0.0958 uM | 0 | 0.08 | 2.44 | 5.94 | 2.65 | 2.87 | 6.39 | 4.93 | 4.05 |
| 0.192 uM | 0 | 2.78 | 9.92 | 11.42 | 11.74 | 10.72 | 14.89 | 10.23 | 6 |
| 0.383 uM | 0 | 0.01 | 4.64 | 6.76 | 3.34 | 2.41 | 2.86 | 0.25 | −2.11 |
| 0.766 uM | 0 | 1.14 | 8.63 | 8.58 | 6.74 | 7.83 | 9.22 | 6.31 | 2.95 |

TABLE 3C
Synergy Scores using the Loewe additivity model for Compound I and
Adagrasib combination treatments on SW1463 (rectum) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.0479 | uM | 0 | 3.14 | 5.52 | 13.8 | 17.83 | 19.84 | 21.89 | 22.37 | 18.53 |
| 0.0958 | uM | 0 | 6.95 | 7.88 | 13.47 | 16.18 | 18.56 | 22.91 | 21.95 | 19.53 |
| 0.192 | uM | 0 | 2.71 | 8.42 | 12.16 | 18.97 | 20.94 | 27.46 | 24.92 | 20.04 |
| 0.383 | uM | 0 | 3.44 | 6.84 | 10.96 | 13.32 | 15.35 | 18.61 | 18.71 | 16.03 |
| 0.766 | uM | 0 | 4.01 | 10.39 | 12.21 | 15.76 | 19.87 | 24.55 | 25.29 | 22.61 |

### Calu-1 (Lung Cancer) Cell Lines

TABLE 4A
Average Inhibition (%) (N = 3) of Compound I and Adagrasib as
single agent and combination treatments on Calu-1 (Lung) cell lines.

| Ave of | | | | | | | | | | |
|---|---|---|---|---|---|---|---|---|---|---|
| Inhibition | | Concentration of Compound I (uM) | | | | | | | | |
| (%) | | 10.0000 | 3.3333 | 1.1111 | 0.3704 | 0.1235 | 0.0412 | 0.0137 | 0.0046 | 0.00 |
| Conc. of | 4.2160 | 95.15% | 92.38% | 89.24% | 86.37% | 83.58% | 81.70% | 79.19% | 77.25% | 72.91% |
| Adagrasib | 2.1080 | 81.73% | 77.49% | 72.33% | 68.59% | 65.50% | 62.41% | 59.11% | 56.51% | 52.82% |
| (uM) | 1.0540 | 69.68% | 64.84% | 58.63% | 49.29% | 44.64% | 38.84% | 35.66% | 31.06% | 25.22% |
| | 0.5270 | 60.78% | 55.45% | 44.48% | 33.11% | 25.40% | 20.68% | 17.66% | 13.02% | 8.85% |
| | 0.2635 | 58.96% | 52.28% | 42.25% | 31.45% | 25.84% | 19.81% | 17.04% | 13.10% | 10.21% |
| | 0.0000 | 22.56% | 11.61% | 31.01% | 3.90% | −0.27% | −3.30% | −5.30% | −8.39% | −6.66% |

TABLE 4B
Synergy Scores using the Bliss independence model for Compound I
and Adagrasib combination treatments on Calu-1 (Lung) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.264 | uM | 0 | 10.42 | 11.59 | 12.57 | 15.87 | 17.74 | 4.19 | 31.64 | 28.49 |
| 0.527 | uM | 0 | 11.82 | 13.64 | 14.84 | 16.79 | 20.7 | 7.36 | 36.02 | 31.37 |
| 1.05 | uM | 0 | 12.11 | 14.39 | 16.09 | 19.62 | 21.15 | 10.22 | 30.93 | 27.58 |
| 2.11 | uM | 0 | 7.65 | 8.79 | 11.15 | 12.8 | 13.94 | 4.88 | 19.19 | 18.27 |
| 4.22 | uM | 0 | 6.61 | 7.72 | 9.69 | 10.74 | 12.41 | 7.93 | 16.33 | 16.13 |

TABLE 4C
Synergy Scores using the Loewe additivity model for Compound I and
Adagrasib combination treatments on Calu-1 (Lung) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.264 | uM | 0 | 4.53 | 8.44 | 11.08 | 16.4 | 11.17 | 8.06 | −17.76 | −19.18 |
| 0.527 | uM | 0 | 1.63 | 6.08 | 8.43 | 10.12 | 11.39 | 2.47 | −15.9 | −17.4 |
| 1.05 | uM | 0 | 6.78 | 11.1 | 13.44 | 16.66 | 13.56 | 4.22 | −8.45 | −8.58 |
| 2.11 | uM | 0 | 3.12 | 5.54 | 8.31 | 9.85 | 8.93 | 4.75 | 1.96 | 3.36 |
| 4.22 | uM | 0 | 4.49 | 6.41 | 8.83 | 10.44 | 12.53 | 13.89 | 15.02 | 16.64 |

### NCI-H358 (Lung) Cancer Cell Lines

TABLE 5A
Average Inhibition (%) of Compound I and Adagrasib as single agent
and combination treatments on NCI-H358 (Lung) cell lines.

| Ave of | | | | | | | | | | |
|---|---|---|---|---|---|---|---|---|---|---|
| Inhibition | | Concentration of Compound I (uM) | | | | | | | | |
| (%) | | 10.0000 | 3.3333 | 1.1111 | 0.3704 | 0.1235 | 0.0412 | 0.0137 | 0.0046 | 0.00 |
| Conc. of | 0.0776 | 96.00% | 94.40% | 92.17% | 90.30% | 87.58% | 82.83% | 78.38% | 73.15% | 68.23% |
| Adagrasib | 0.0388 | 92.97% | 89.56% | 86.36% | 81.39% | 76.50% | 70.34% | 62.56% | 58.99% | 51.36% |
| (uM) | 0.0194 | 89.84% | 85.29% | 79.35% | 71.41% | 62.78% | 54.56% | 43.53% | 38.52% | 30.10% |
| | 0.0097 | 82.84% | 76.28% | 66.33% | 53.00% | 41.75% | 34.38% | 23.20% | 19.50% | 18.32% |
| | 0.0049 | 77.40% | 67.89% | 53.34% | 39.06% | 29.63% | 21.23% | 12.06% | 7.43% | 5.58% |
| | 0.0000 | 61.00% | 50.36% | 38.90% | 25.04% | 17.08% | 10.78% | 4.10% | 0.76% | −1.85% |

TABLE 5B
Synergy Scores using the Bliss independence model for Compound I and
Adagrasib combination treatments on NCI-H358 (Lung) cell lines.

| | | Compound I | | | | | | | | |
|---|---|---|---|---|---|---|---|---|---|---|
| | | | 0.0046 | 0.0137 | 0.0412 | 0.1235 | 0.3704 | 1.1111 | 3.3333 | 10.0000 |
| Adagrasib | | 0 | uM | uM | uM | uM | uM | uM | uM | uM |
| 0 | uM | 0 | 0 | 0 | 0 | 0 | 0 | 0 | 0 | 0 |
| 0.00485 | uM | 0 | 1.13 | 2.61 | 5.47 | 7.92 | 9.84 | 11.02 | 14.76 | 14.23 |
| 0.0097 | uM | 0 | 0.56 | 1.53 | 7.26 | 9.49 | 14.24 | 16.23 | 16.83 | 14.7 |
| 0.0194 | uM | 0 | 7.89 | 10.56 | 16.92 | 20.74 | 23.81 | 22.06 | 19.98 | 17.1 |
| 0.0388 | uM | 0 | 7.25 | 9.2 | 13.73 | 16.83 | 17.85 | 16.08 | 13.7 | 11.93 |
| 0.0776 | uM | 0 | 4.68 | 8.85 | 11.18 | 13.92 | 14.11 | 11.59 | 10.17 | 8.39 |

TABLE 5C
Synergy Scores using the Loewe additivity model for Compound I and
Adagrasib combination treatments on NCI-H358 (Lung) cell lines.

| | | Compound I | | | | | | | | |
|---|---|---|---|---|---|---|---|---|---|---|
| | | 0.0000 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.00485 | uM | 0 | −0.72 | 1.47 | 6.43 | 8.48 | 9.27 | 13.06 | 16.41 | 15.41 |
| 0.0097 | uM | 0 | 2.08 | 4.64 | 13.22 | 15.82 | 19.81 | 23.79 | 23.42 | 20.06 |
| 0.0194 | uM | 0 | 6.41 | 11.08 | 21.15 | 27.08 | 31.2 | 32.21 | 29.63 | 25.48 |
| 0.0388 | uM | 0 | 8.66 | 12.16 | 19.72 | 25.28 | 28.63 | 30.35 | 28.35 | 25.43 |
| 0.0776 | uM | 0 | 4.65 | 9.87 | 14.3 | 18.95 | 21.43 | 22.66 | 23.37 | 22.27 |

### SW837 (Rectal Cancer) Cell Lines

TABLE 6A
Average Inhibition (%) (N = 3) of Compound I and Adagrasib as
single agent and combination treatments on SW837 (Rectum) cell lines.

| Ave of | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 0.2530 | 72.68% | 72.27% | 72.48% | 71.74% | 71.45% | 68.96% | 63.73% | 59.70% | 55.03% |
| Adagrasib | 0.1265 | 68.19% | 68.20% | 67.26% | 66.98% | 65.54% | 63.33% | 59.17% | 54.89% | 48.87% |
| (uM) | 0.0633 | 68.84% | 67.53% | 66.54% | 66.09% | 62.93% | 62.20% | 55.68% | 52.94% | 46.69% |
| | 0.0316 | 63.98% | 64.10% | 64.53% | 62.73% | 60.59% | 58.84% | 52.09% | 48.02% | 41.56% |
| | 0.0158 | 62.37% | 61.19% | 60.91% | 59.70% | 56.32% | 52.79% | 47.24% | 42.42% | 35.25% |
| | 0.0000 | 27.54% | 21.82% | 18.97% | 15.92% | 9.71% | 8.04% | −0.64% | −3.30% | −3.83% |

TABLE 6B
Synergy Scores using the Bliss independence model for Compound I and
Adagrasib combination treatments on SW837 (Rectum) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.0158 | uM | 0 | 9.31 | 12.39 | 12.33 | 14.78 | 14.14 | 13.37 | 11.81 | 9.29 |
| 0.0316 | uM | 0 | 8.39 | 10.91 | 12.59 | 13.36 | 11.87 | 11.88 | 9.79 | 6.32 |
| 0.0633 | uM | 0 | 8.01 | 9.33 | 11.23 | 11.06 | 10.91 | 9.74 | 9.21 | 7.47 |
| 0.126 | uM | 0 | 7.7 | 10.62 | 10.35 | 11.7 | 9.96 | 8.69 | 8.17 | 5.24 |
| 0.253 | uM | 0 | 6.16 | 8.99 | 10.32 | 12.06 | 9.55 | 8.92 | 7.43 | 5.27 |

TABLE 6C
Synergy Scores using the Loewe additivity model for Compound I and
Adagrasib combination treatments on SW837 (Rectum) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.0158 | uM | 0 | 6.76 | 11.57 | 17.12 | 20.66 | 24.03 | 25.23 | 25.48 | 26.56 |
| 0.0316 | uM | 0 | 7.02 | 11.09 | 17.84 | 19.59 | 21.73 | 23.53 | 23.1 | 22.97 |
| 0.0633 | uM | 0 | 6.51 | 8.42 | 12.97 | 10.22 | 9.01 | 5.26 | 2.8 | 1.49 |
| 0.126 | uM | 0 | 4.26 | 8.13 | 11.24 | 11.23 | 9.26 | 5.78 | 3.41 | 0.83 |
| 0.253 | uM | 0 | 5.23 | 9.08 | 13.8 | 15.05 | 13.01 | 10.65 | 7.38 | 5.3 |

### SW756 (Cervical Cancer) Cell Lines

TABLE 7A
Average Inhibition (%) (N = 3) of Compound I and Adagrasib as
single agent and combination treatments on SW756 (Cervix) cell lines.

| Ave | | | | | | | | | | |
|---|---|---|---|---|---|---|---|---|---|---|
| Inhibition | | Concentration of Compound I (uM) | | | | | | | | |
| (%) | | 10.0000 | 3.3333 | 1.1111 | 0.3704 | 0.1235 | 0.0412 | 0.0137 | 0.0046 | 0.00 |
| Conc. of | 2.8698 | 92.84% | 90.45% | 87.10% | 85.05% | 82.83% | 81.31% | 80.27% | 79.36% | 76.42% |
| Adagrasib | 1.4349 | 83.02% | 79.86% | 76.38% | 72.21% | 68.91% | 67.18% | 63.47% | 61.44% | 58.51% |
| (uM) | 0.7175 | 73.55% | 69.60% | 65.31% | 59.30% | 54.43% | 52.48% | 49.92% | 48.15% | 42.14% |
| | 0.3587 | 66.61% | 60.11% | 52.38% | 44.76% | 40.65% | 33.87% | 32.19% | 29.50% | 25.77% |
| | 0.1794 | 60.78% | 56.81% | 49.62% | 39.66% | 35.74% | 29.59% | 27.68% | 26.87% | 22.91% |
| | 0.0000 | 34.59% | 21.65% | 14.30% | 8.26% | 4.83% | 1.55% | −0.86% | −1.03% | −3.85% |

TABLE 7B
Synergy Scores using the Bliss independence model for Compound I and
Adagrasib combination treatments on SW756 (Cervix) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.179 | uM | 0 | 4.75 | 5.44 | 5.48 | 9.11 | 10.38 | 15.69 | 17.21 | 11.21 |
| 0.359 | uM | 0 | 4.5 | 7.07 | 6.95 | 11.29 | 12.86 | 16.01 | 18.28 | 15.17 |
| 0.717 | uM | 0 | 6.61 | 8.28 | 9.44 | 9.5 | 12.38 | 14.9 | 14.94 | 11.4 |
| 1.43 | uM | 0 | 3.36 | 5.32 | 8.03 | 8.4 | 10.28 | 11.94 | 12.38 | 10.17 |
| 2.87 | uM | 0 | 3.18 | 4.05 | 4.52 | 5.27 | 6.68 | 7.3 | 8.92 | 8.26 |

TABLE 7C
Synergy Scores using the Loewe additivity model for Compound I and
Adagrasib combination treatments on SW756 (Cervix) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.179 | uM | 0 | 4.89 | 5.69 | 7.55 | 13.57 | 17.05 | 25.52 | 28.04 | 22.61 |
| 0.359 | uM | 0 | 1.72 | 4.4 | 6.01 | 12.61 | 16.18 | 22.23 | 25.93 | 24.43 |
| 0.717 | uM | 0 | 7.71 | 9.46 | 11.98 | 13.81 | 18.33 | 23.35 | 25.07 | 23.5 |
| 1.43 | uM | 0 | 2.04 | 4.06 | 7.76 | 9.45 | 12.64 | 16.48 | 19.03 | 19.8 |
| 2.87 | uM | 0 | 3.19 | 4.1 | 5.13 | 6.65 | 8.84 | 10.81 | 13.93 | 15.66 |

### NCI-H2122 (Lung Cancer) Cell Lines

TABLE 8A
Average Inhibition (%) (N = 3) of Compound I and Adagrasib as
single agent and combination treatments on NCI-H2122 (Lung) cell lines.

| Ave of | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 0.9828 | 99.19% | 99.01% | 98.97% | 98.62% | 97.97% | 96.94% | 94.11% | 89.66% | 80.35% |
| Adagrasib | 0.4914 | 99.01% | 98.73% | 98.24% | 97.89% | 96.97% | 94.83% | 90.32% | 83.58% | 67.90% |
| (uM) | 0.2457 | 98.91% | 98.49% | 98.04% | 97.31% | 95.70% | 93.32% | 86.92% | 77.09% | 64.64% |
| | 0.1229 | 98.39% | 97.76% | 96.99% | 95.87% | 93.47% | 88.16% | 77.35% | 67.11% | 51.33% |
| | 0.0614 | 97.62% | 96.22% | 94.19% | 91.74% | 86.44% | 76.12% | 61.58% | 47.04% | 32.70% |
| | 0.0000 | 63.19% | 52.55% | 45.56% | 36.51% | 23.99% | 13.59% | 2.34% | −2.13% | −5.36% |

TABLE 8B
Synergy Scores using the Bliss independence model for Compound I and
Adagrasib combination treatments on NCI-H2122 (Lung) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.0614 | uM | 0 | 15.78 | 27.3 | 34.27 | 37.6 | 34.48 | 30.83 | 28.16 | 22.4 |
| 0.123 | uM | 0 | 16.83 | 24.88 | 30.22 | 30.47 | 26.77 | 23.48 | 20.85 | 16.31 |
| 0.246 | uM | 0 | 13.2 | 21.45 | 23.88 | 22.57 | 19.76 | 17.29 | 15.26 | 11.92 |
| 0.491 | uM | 0 | 16.36 | 21.66 | 22.56 | 21.37 | 18.27 | 15.72 | 13.96 | 10.82 |
| 0.983 | uM | 0 | 9.73 | 13.3 | 13.92 | 12.91 | 11.1 | 9.66 | 8.33 | 6.42 |

TABLE 8C
Synergy Scores using the Loewe additivity model for Compound I and
Adagrasib combination treatments on NCI-H2122 (Lung) cell lines.

| | | Compound I | | | | | | | | |
|---|---|---|---|---|---|---|---|---|---|---|
| | | 0 | 0.0046 | 0.0137 | 0.0412 | 0.1235 | 0.3704 | 1.1111 | 3.3333 | 10.0000 |
| Adagrasib | | uM | uM | uM | uM | uM | uM | uM | uM | uM |
| 0 | uM | 0 | 0 | 0 | 0 | 0 | 0 | 0 | 0 | 0 |
| 0.0614 | uM | 0 | 13.35 | 27.17 | 39.95 | 46.82 | 46.87 | 42.95 | 38.51 | 34.15 |
| 0.123 | uM | 0 | 16.71 | 26.84 | 37.37 | 41.91 | 42.55 | 40.54 | 37.15 | 33.4 |
| 0.246 | uM | 0 | 14.03 | 23.85 | 30.22 | 32.49 | 33.82 | 33.84 | 32.82 | 31.02 |
| 0.491 | uM | 0 | 11.53 | 18.27 | 22.78 | 24.92 | 25.82 | 26.11 | 26.44 | 26.31 |
| 0.983 | uM | 0 | 11.48 | 15.93 | 18.76 | 19.79 | 20.44 | 20.78 | 20.83 | 21.01 |

### NCI-H1373 (Lung Cancer) Cell Lines

TABLE 9A
Average Inhibition (%) (N = 3) of Compound I and Adagrasib as
single agent and combination treatments on NCI-H1373 (Lung) cell lines.

| Ave | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 4.8696 | 94.57% | 96.31% | 95.48% | 92.68% | 91.77% | 91.18% | 88.72% | 85.52% | 81.51% |
| Adagrasib | 2.4348 | 85.05% | 77.43% | 67.62% | 59.64% | 56.51% | 55.52% | 51.50% | 49.82% | 44.31% |
| (uM) | 1.2174 | 64.22% | 52.32% | 41.20% | 32.34% | 28.64% | 22.42% | 20.72% | 17.40% | 19.60% |
| | 0.6087 | 50.45% | 35.18% | 23.60% | 10.43% | 5.14% | 3.99% | 3.06% | 1.23% | 1.70% |
| | 0.3044 | 44.73% | 29.00% | 18.51% | 5.40% | 0.81% | −0.79% | 0.70% | 0.02% | 3.40% |
| | 0.0000 | 8.28% | −9.75% | −4.70% | −5.69% | −6.17% | −5.53% | −6.68% | −3.29% | −0.65% |

TABLE 9B
Synergy Scores using the Bliss independence model for Compound I and
Adagrasib combination treatments on NCI-H1373 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.304 uM | 0 | −0.2 | 3.75 | 1.15 | 3.37 | 7.49 | 19.64 | 35.01 | 33.33 |
| 0.609 uM | 0 | 2.77 | 7.93 | 7.73 | 9.51 | 14.32 | 26.52 | 43.07 | 40.61 |
| 1.22 uM | 0 | 0.45 | 6.5 | 7.27 | 13.99 | 17.31 | 25.37 | 40.55 | 37.96 |
| 2.43 uM | 0 | 7.35 | 10.92 | 14.29 | 15.64 | 18.5 | 25.93 | 38.55 | 36.13 |
| 4.87 uM | 0 | 4.61 | 8.44 | 10.69 | 11.4 | 12.21 | 14.83 | 16.6 | 11.52 |

TABLE 9C
Synergy Scores using the Loewe additivity model for Compound I and
Adagrasib combination treatments on NCI-H1373 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.304 uM | 0 | −1.33 | −0.65 | −2.15 | −0.57 | 3.93 | 16.74 | 25.72 | 6.51 |
| 0.609 uM | 0 | −4.13 | −2.3 | −1.41 | −0.33 | 4.68 | 16.95 | 24.45 | −6.95 |
| 1.22 uM | 0 | −0.04 | 3.25 | 4.88 | 10.88 | 13.92 | 20.6 | 23.07 | −15.96 |
| 2.43 uM | 0 | 4.79 | 6.43 | 10.33 | 10.97 | 13.02 | 17.63 | 16.09 | −14.74 |
| 4.87 uM | 0 | 4.12 | 7.29 | 9.65 | 9.96 | 9.99 | 10.19 | 3.36 | −17.25 |

### NCI-H1792 (Lung Cancer) Cell Lines

TABLE 10A
Average Inhibition (%) (N = 3) of Compound I and Adagrasib as
single agent and combination treatments on NCI-H1792 (Lung) cell lines.

| Ave of | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 2.3504 | 92.92% | 92.43% | 91.35% | 88.44% | 85.89% | 82.68% | 78.82% | 75.87% | 70.71% |
| Adagrasib | 1.1752 | 78.51% | 75.10% | 72.15% | 69.03% | 66.94% | 63.73% | 59.90% | 56.29% | 49.99% |
| (uM) | 0.5876 | 71.64% | 67.33% | 64.03% | 61.37% | 57.57% | 55.19% | 50.67% | 46.83% | 39.18% |
| | 0.2938 | 67.87% | 62.36% | 57.45% | 54.43% | 50.98% | 47.41% | 44.25% | 38.31% | 32.03% |
| | 0.1469 | 65.06% | 59.73% | 52.31% | 48.49% | 47.14% | 42.18% | 39.97% | 33.90% | 26.53% |
| | 0.0000 | 34.00% | 22.38% | 12.24% | 7.05% | 6.64% | 2.27% | −1.40% | −7.35% | −8.87% |

TABLE 10B
Synergy Scores using the Bliss independence model for Compound I and
Adagrasib combination treatments on NCI-H1792 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.147 uM | 0 | 12.76 | 14.47 | 13.98 | 15.72 | 16.79 | 16.78 | 16.76 | 13.55 |
| 0.294 uM | 0 | 11.28 | 13.18 | 13.84 | 14.43 | 17.62 | 17.11 | 15.12 | 12.73 |
| 0.588 uM | 0 | 12.12 | 12.35 | 14.63 | 14.35 | 17.91 | 17.41 | 14.54 | 11.78 |
| 1.18 uM | 0 | 9.97 | 10.6 | 12.6 | 13.63 | 15.51 | 16.03 | 13.91 | 11.52 |
| 2.35 uM | 0 | 7.31 | 8.52 | 11.31 | 13.23 | 15.66 | 17.05 | 15.17 | 12.25 |

TABLE 10C
Synergy Scores using the Loewe additivity model for Compound I and
Adagrasib combination treatments on NCI-H1792 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.147 uM | 0 | 6.67 | 12.74 | 14.93 | 19.82 | 20.98 | 24.14 | 29.36 | 28.55 |
| 0.294 uM | 0 | 7 | 12.94 | 16.06 | 19.56 | 22.78 | 25.12 | 27.98 | 28.34 |
| 0.588 uM | 0 | 8.26 | 12.1 | 16.59 | 18.9 | 22.52 | 24.61 | 26.31 | 26.73 |
| 1.18 uM | 0 | 5.37 | 8.98 | 12.8 | 15.98 | 17.95 | 20.75 | 22.8 | 23.89 |
| 2.35 uM | 0 | 5.43 | 8.38 | 12.24 | 15.43 | 17.95 | 20.77 | 21.56 | 21.23 |

### NCI-H23 (Lung Cancer) Cell Lines

TABLE 11A
Average Inhibition (%) (N = 3) of Compound I and Adagrasib as
single agent and combination treatments on NCI-H23 (Lung) cell lines.

| Ave | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 1.0116 | 86.39% | 84.61% | 81.84% | 79.59% | 76.57% | 74.08% | 69.34% | 68.05% | 62.78% |
| Adagrasib | 0.5058 | 80.23% | 77.67% | 73.92% | 71.13% | 67.39% | 64.26% | 60.12% | 57.53% | 53.03% |
| (uM) | 0.2529 | 78.91% | 76.15% | 72.99% | 69.47% | 66.70% | 61.40% | 58.98% | 53.76% | 50.92% |
| | 0.1265 | 76.58% | 71.60% | 69.73% | 63.62% | 59.29% | 56.87% | 51.49% | 46.73% | 42.26% |
| | 0.0632 | 74.85% | 68.64% | 65.35% | 59.63% | 53.72% | 50.71% | 45.08% | 39.95% | 38.43% |
| | 0.0000 | 37.19% | 28.60% | 23.40% | 14.90% | 15.43% | 12.05% | 7.11% | 6.06% | 0.92% |

TABLE 11B
Synergy Scores using the Bliss independence model for Compound I and
Adagrasib combination treatments on NCI-H23 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.0632 uM | 0 | −2.21 | 2.28 | 4.86 | 5.78 | 12.03 | 12.51 | 12.6 | 13.52 |
| 0.126 uM | 0 | 0.97 | 5.12 | 7.65 | 8.12 | 12.76 | 13.96 | 12.82 | 12.84 |
| 0.253 uM | 0 | −0.14 | 4.57 | 4.57 | 8.2 | 11.24 | 10.58 | 11.19 | 9.73 |
| 0.506 uM | 0 | 1.65 | 3.75 | 5.57 | 7.11 | 11.1 | 9.9 | 11.21 | 9.73 |
| 1.01 uM | 0 | 3.02 | 3.92 | 6.81 | 8.05 | 11.26 | 10.35 | 11.18 | 9.77 |

TABLE 11C
Synergy Scores using the Loewe additivity model for Compound I and
Adagrasib combination treatments on NCI-H23 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.0632 uM | 0 | 1.63 | 6.76 | 12.37 | 15.34 | 21.15 | 26.53 | 28.9 | 32.9 |
| 0.126 uM | 0 | 3.62 | 8.37 | 13.74 | 16.15 | 20.41 | 26.34 | 27.68 | 31.35 |
| 0.253 uM | 0 | 4.96 | 10.18 | 12.6 | 17.89 | 20.63 | 24.07 | 27.01 | 29.17 |
| 0.506 uM | 0 | 2.33 | 4.92 | 9.06 | 12.19 | 15.93 | 18.69 | 22.39 | 24.79 |
| 1.01 uM | 0 | 6.05 | 7.34 | 12.08 | 14.57 | 17.58 | 19.84 | 22.6 | 24.38 |

### MIA PaCa-2 (Pancreatic Cancer) Cell Lines

TABLE 12A
Average Inhibition (%) (N = 3) of Compound I and Adagrasib as single
agent and combination treatments on MIA PaCa-2 (Pancreas) cell lines.

| Ave | | | | | | | | | | |
|---|---|---|---|---|---|---|---|---|---|---|
| Inhibition | Concentration of Compound I (uM) | | | | | | | | | |
| (%) | | 10.0000 | 3.3333 | 1.1111 | 0.3704 | 0.1235 | 0.0412 | 0.0137 | 0.0046 | 0.00 |
| Conc. of | 2.2484 | 84.38% | 81.98% | 79.67% | 77.82% | 75.25% | 73.17% | 69.94% | 66.39% | 63.69% |
| Adagrasib | 1.1242 | 79.60% | 76.66% | 73.85% | 71.00% | 67.38% | 65.13% | 60.38% | 58.83% | 54.56% |
| (uM) | 0.5621 | 79.17% | 76.75% | 73.04% | 69.09% | 65.53% | 63.23% | 58.56% | 55.21% | 52.77% |
| | 0.2811 | 77.14% | 74.05% | 69.95% | 65.58% | 61.09% | 56.98% | 53.61% | 49.02% | 46.50% |
| | 0.1405 | 76.78% | 72.50% | 67.84% | 62.28% | 57.58% | 53.18% | 48.84% | 46.57% | 43.64% |
| | 0.0000 | 30.59% | 13.86% | 1.46% | −5.32% | −9.50% | −8.58% | −10.33% | −5.76% | −3.22% |

TABLE 12B
Synergy Scores using the Bliss independence model for Compound I and
Adagrasib combination treatments on MIA PaCa-2 (Pancreas) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.141 uM | 0 | 6.17 | 11.02 | 14.37 | 19.29 | 21.63 | 23.38 | 21.04 | 15.9 |
| 0.281 uM | 0 | 5.6 | 12.63 | 15.07 | 19.68 | 21.92 | 22.67 | 20.13 | 14.27 |
| 0.562 uM | 0 | 5.16 | 10.66 | 14.51 | 17.24 | 18.82 | 19.58 | 17.43 | 11.95 |
| 1.12 uM | 0 | 6.89 | 10.52 | 14.47 | 17.14 | 18.86 | 18.63 | 15.8 | 11.14 |
| 2.25 uM | 0 | 4.79 | 9.99 | 12.59 | 15 | 16.06 | 15.45 | 13.25 | 9.58 |

TABLE 12C
Synergy Scores using the Loewe additivity model for Compound I and Adagrasib
combination treatments on MIA PaCa-2 (Pancreas) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.141 uM | 0 | 2.74 | 5.01 | 9.35 | 13.75 | 18.43 | 23.95 | 28.47 | 32.37 |
| 0.281 uM | 0 | 2.18 | 6.77 | 10.14 | 14.25 | 18.73 | 23.1 | 27.2 | 30.27 |
| 0.562 uM | 0 | 4.16 | 7.4 | 11.74 | 13.11 | 14.25 | 12.93 | 7.75 | −0.98 |
| 1.12 uM | 0 | 2.37 | 3.85 | 8.39 | 10.03 | 11.98 | 10.83 | 6.15 | −1.11 |
| 2.25 uM | 0 | 3.29 | 6.79 | 9.5 | 11.61 | 13.14 | 12.31 | 8.94 | 2.65 |

### HCC44 (Lung Cancer) Cell Lines

TABLE 13A
Average Inhibition (%) (N = 3) of Compound I and Adagrasib
as single agent and combination treatments on HCC44 (Lung) cell lines.

| Ave | Concentration of Compound I (uM) | | | | | | | | | |
|---|---|---|---|---|---|---|---|---|---|---|
| Inhibition (%) | | 10.0000 | 3.3333 | 1.1111 | 0.3704 | 0.1235 | 0.0412 | 0.0137 | 0.0046 | 0.00 |
| Conc. of | 6.4456 | 100.36% | 100.07% | 99.19% | 97.01% | 95.03% | 93.68% | 91.96% | 89.60% | 86.44% |
| Adagrasib | 3.2228 | 99.32% | 92.92% | 74.54% | 66.28% | 60.96% | 56.66% | 51.24% | 47.59% | 43.28% |
| (uM) | 1.6114 | 90.14% | 74.23% | 55.97% | 44.80% | 40.35% | 36.21% | 31.20% | 28.78% | 25.88% |
| | 0.8057 | 81.25% | 67.95% | 47.67% | 38.64% | 34.54% | 32.03% | 28.27% | 24.00% | 22.50% |
| | 0.4029 | 80.42% | 65.18% | 44.18% | 37.37% | 34.01% | 30.09% | 26.98% | 22.45% | 22.83% |
| | 0.0000 | 43.68% | 33.10% | 19.73% | 10.10% | 1.74% | −1.38% | −4.27% | −3.82% | −2.14% |

TABLE 13B
Synergy Scores using the Bliss independence model for Compound I
and Adagrasib combination treatments on HCC44 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.403 uM | 0 | 2.58 | 7.45 | 8.33 | 9.84 | 6.75 | 6.12 | 16.8 | 23.88 |
| 0.806 uM | 0 | 4.46 | 9.09 | 10.6 | 10.69 | 8.31 | 9.87 | 19.8 | 24.9 |
| 1.61 uM | 0 | 5.73 | 8.48 | 11.35 | 13.19 | 11.43 | 15.47 | 23.82 | 31.88 |
| 3.22 uM | 0 | 6.48 | 10.38 | 14.16 | 16.69 | 17.27 | 20.07 | 30.86 | 31.26 |
| 6.45 uM | 0 | 3.68 | 6.1 | 7.43 | 8.35 | 9.2 | 10.07 | 9.14 | 8 |

TABLE 13C
Synergy Scores using the Loewe additivity model for Compound I and
Adagrasib combination treatments on HCC44 (Lung) cell lines.

| | Compound I | | | | | | | | |
|---|---|---|---|---|---|---|---|---|---|
| Adagrasib | 0 uM | 0.0046 uM | 0.0137 uM | 0.0412 uM | 0.1235 uM | 0.3704 uM | 1.1111 uM | 3.3333 uM | 10.0000 uM |
| 0 uM | 0 | 0 | 0 | 0 | 0 | 0 | 0 | 0 | 0 |
| 0.403 uM | 0 | −0.06 | 4.46 | 7.56 | 11.48 | 14.76 | 19.8 | 30.4 | 35.69 |
| 0.806 uM | 0 | 1.1 | 5.36 | 9.09 | 11.5 | 15.15 | 20.6 | 31.27 | 35.46 |
| 1.61 uM | 0 | 2.97 | 5.34 | 10.2 | 13.87 | 16.73 | 23.17 | 33.53 | 42.09 |
| 3.22 uM | 0 | 4.28 | 7.88 | 13.16 | 17.08 | 21.4 | 27.53 | 42.71 | 45.98 |
| 6.45 uM | 0 | 3.12 | 5.4 | 6.88 | 7.51 | 7.46 | 4.49 | −3.96 | −11.59 |

What is claimed is:

1. A pharmaceutical combination comprising a therapeutically effective amount of a compound that forms a covalent bond with a kinase or pseudokinase, or a pharmaceutically acceptable salt thereof, and at least one additional therapeutically active agent.

2. The pharmaceutical combination of claim 1, wherein the kinase is PI3-kinase (PI3K).

3. The pharmaceutical combination of claim 1, wherein the compound that forms a covalent bond with a kinase or pseudokinase is a compound having the formula:
(FCB)a-(L)b-(CLM)c,
or a pharmaceutically acceptable salt thereof,
wherein:
CLM is a covalent linking modality;
L is a linker;
a and c are each independently integers between 1 and 5;
b is an integer between 0 and 5; and
FCB comprises:

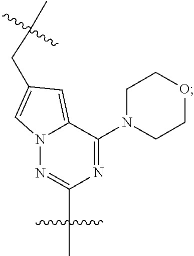 [SMILES: *Cc1cc2c(N3CCOCC3)nc(*)nn2c1]

4. The pharmaceutical combination of claim 3, wherein FCB has the structure: 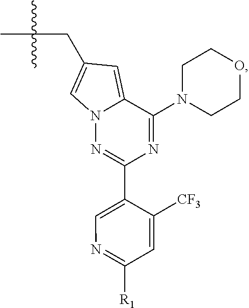
wherein R₁ is selected from the group consisting of:

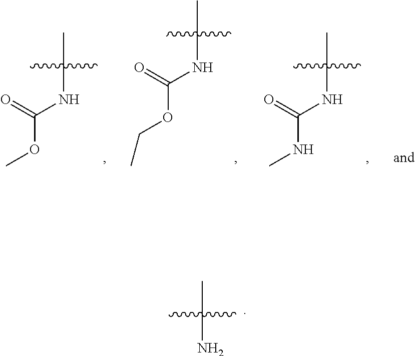

5. The pharmaceutical combination of claim 4, wherein the compound is Compound I:

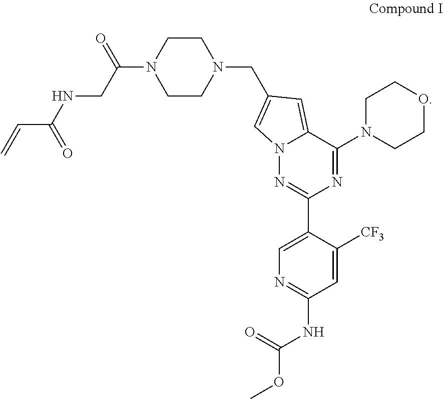 [SMILES: C=CC(=O)NCC(=O)N1CCN(Cc2cc3c(N4CCOCC4)nc(-c4cnc(NC(=O)OC)cc4C(F)(F)F)nn3c2)CC1]

6. The pharmaceutical combination of any one of claims 1-5, wherein the additional therapeutically active agent is a RAS inhibitor or specifically a KRAS inhibitor.

7. The pharmaceutical combination of claim 6, wherein the additional therapeutically active agent is an inhibitor for a KRAS mutant.

8. The pharmaceutical combination of claim 7, wherein KRAS mutant is G12C.

9. The pharmaceutical combination of claim 7, wherein the additional therapeutically active agent covalently binds to KRAS-G12C.

10. The pharmaceutical combination of any one of claims 6-9, wherein the KRAS inhibitor is selected from one or more of the group consisting of sotorasib, adagrasib, ARS-3248, LY3499446, LY3537982, GDC-6036, D-1553, JDQ443, and BI 1823911.

11. The pharmaceutical combination of claim 10, wherein the KRAS inhibitor is selected from one or more of the group consisting of sotorasib and adagrasib.

12. The pharmaceutical combination of claim 11, wherein the KRAS inhibitor is adagrasib.

13. The pharmaceutical combination of claim 12, wherein the dosage amount of adagrasib is the range of about 200 mg to about 2000 mg, or about 400 mg to about 1000 mg, or about 500 mg to about 700 mg.

14. The pharmaceutical combination of claim 13, wherein the dose is administered once, twice, or three times a day.

15. The pharmaceutical combination of any one of claims 1-14, wherein the compound that forms a covalent bond with a kinase or pseudokinase, or a pharmaceutically acceptable salt thereof and the at least one additional therapeutically active agent are in single dosage form or in separate dosage forms.

16. The pharmaceutical combination of claim 15, wherein the separate dosage forms are administered via same mode of administration or different modes of administration.

17. The pharmaceutical combination of claim 16, wherein the separate dosage forms are coadministered via simultaneous administration, sequential administration, overlapping administration, interval administration, continuous administration, or a combination thereof.

18. A method for treating or ameliorating cell proliferation disorder in a subject, the method comprising administering to a subject in need thereof a therapeutically effective amount of a compound that forms a covalent bond with a kinase or pseudokinase or a pharmaceutically acceptable salt thereof, and administering at least one additional therapeutically active agent before, during, or after the subject has been administered compound that forms a covalent bond with a kinase or pseudokinase.

19. The method of claim 18, wherein the kinase is PI3-kinase (PI3K).

20. The method of claim 18, wherein the compound that forms a covalent bond with a kinase or pseudokinase is a compound having the formula:
(FCB)a-(L)b-(CLM)c,
or a pharmaceutically acceptable salt thereof,
wherein:
CLM is a covalent linking modality;
L is a linker;
a and c are each independently integers between 1 and 5;
b is an integer between 0 and 5; and
FCB comprises:

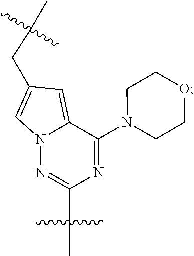 [SMILES: *Cc1cc2c(N3CCOCC3)nc(*)nn2c1]

21. The method of claim 20, wherein FCB has the structure: 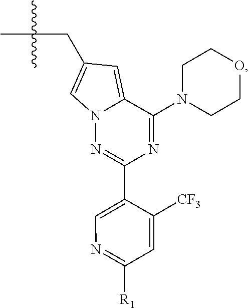
wherein R₁ is selected from the group consisting of:

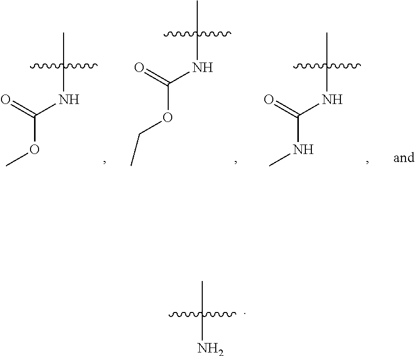

22. The method of claim 21, wherein the compound is Compound I:

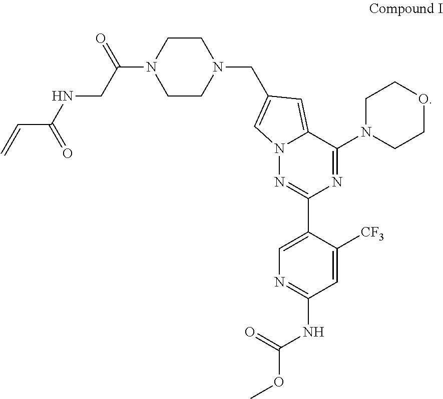 [SMILES: C=CC(=O)NCC(=O)N1CCN(Cc2cc3c(N4CCOCC4)nc(-c4cnc(NC(=O)OC)cc4C(F)(F)F)nn3c2)CC1]

23. The method of any one of claims 18-22, wherein the additional therapeutically active agent is a RAS inhibitor or specifically KRAS inhibitor.

24. The method of claim 23, wherein the additional therapeutically active agent is an inhibitor for a KRAS mutant.

25. The method of claim 24, wherein the KRAS mutant is G12C.

26. The method of claim 25, wherein the additional therapeutically active agent covalently binds to KRAS-G12C.

27. The method of any one of claims 23-26, wherein the KRAS inhibitor is selected from one or more of the group consisting of sotorasib, adagrasib, ARS-3248, LY3499446, LY3537982, GDC-6036, D-1553, JDQ443, and BI 1823911.

28. The method of claim 26, wherein the KRAS inhibitor is selected from one or more of the group consisting of sotorasib and adagrasib.

29. The method of claim 28, wherein the KRAS inhibitor is adagrasib.

30. The method of claim 29, wherein the dosage amount of adagrasib is the range of about 200 mg to about 2000 mg, or about 400 mg to about 1000 mg, or about 500 mg to about 700 mg.

31. The method of claim 30, wherein the dose is administered once, twice, or three times a day.

32. The method any one of claims 18-31, wherein the compound that forms a covalent bond with a kinase or pseudokinase, or a pharmaceutically acceptable salt thereof and the at least one additional therapeutically active agent are in single dosage form or in separate dosage forms.

33. The pharmaceutical combination of claim 32, wherein the separate dosage forms are administered via same mode of administration or different modes of administration.

34. The pharmaceutical combination of claim 33, wherein the separate dosage forms are coadministered via simultaneous administration, sequential administration, overlapping administration, interval administration, continuous administration, or a combination thereof.

35. The method of any one of claims 18-34, wherein the cell proliferation disorder is cancer.

36. The method of claim 35, wherein the cancer is selected from the group consisting of: heme cancer, colorectal cancer, ovarian cancer, breast cancer, cervical cancer, lung cancer, liver cancer, colon cancer, pancreatic cancer, cancer of the lymph nodes, colon cancer, small intestine, prostate cancer, brain cancer, cholangiocarcinoma, gallbladder carcinoma, cancer of the head and neck, bone cancer, Ewing's sarcoma, skin cancer, kidney cancer, and cancer of the heart.

37. The method of claim 36, wherein the cancer is selected from the group consisting of wherein the cancer is selected from the group consisting of breast cancer, ovarian cancer, pancreatic cancer, colorectal cancer, colon cancer, small intestine and lung cancer.

38. The method of any one of claims 18-37, wherein the subject is human.

39. The pharmaceutical combination of claim 1, which is a pharmaceutical formulation further comprising a pharmaceutically acceptable excipient or a pharmaceutically acceptable carrier.
